(19) United States
(12) Patent Application Publication
(10) Pub. No.: US 2024/0417408 A1
(43) Pub. Date: Dec. 19, 2024

(54) SMALL MOLECULE INHIBITORS OF KRAS G12C MUTANT

(71) Applicant: MERCK SHARP & DOHME CORP., Rahway, NJ (US)

(72) Inventors: Kazuaki SHIBATA, Ibaraki (JP); Yu KOBAYAKAWA, Ibaraki (JP); Tadashi IMAOKA, Ibaraki (JP); Toshihiro SAKAMOTO, Ibaraki (JP); Risako MIURA, Ibaraki (JP); Elisabeth HENNESSY, Weston, MA (US); Juan DEL POZO, Brookline, MA (US)

(21) Appl. No.: 18/564,799

(22) PCT Filed: May 27, 2022
(86) PCT No.: PCT/US2022/031256

§ 371 Date: Nov. 28, 2023

Related U.S. Application Data
(60) Provisional application No. 63/194,799, filed on May 28, 2021.

(51) Int. Cl. C07D 491/052; A61K 31/519; A61K 31/5377; A61K 31/55; A61K 45/06; A61P 35/00; C07D 519/00

(52) U.S. Cl. CPC C07D 491/052; A61K 31/519; A61K 31/5377; A61K 31/55; A61K 45/06; A61P 35/00; C07D 519/00

(57) ABSTRACT

Compounds of Formula (I) or their pharmaceutically acceptable salts can inhibit the G12C mutant of Kirsten rat sarcoma (KRAS) protein and are expected to have utility as therapeutic agents, for example, for treating cancer. The disclosure also provides pharmaceutical compositions which comprise compounds of Formula (I) or pharmaceutically acceptable salts thereof. The disclosure also relates to methods for use of the compounds or their pharmaceutically acceptable salts in the therapy and prophylaxis of cancer and for preparing pharmaceuticals for this purpose.

### CROSS REFERENCE TO RELATED APPLICATION

The present application claims the benefit of U.S. Provisional Application No. 63/194,799, filed May 28, 2021, the entirety of which is incorporated by reference herein.

### FIELD OF THE INVENTION

The present invention relates to heterocyclic compounds that inhibit the G12C mutant of Kirsten rat sarcoma (KRAS) protein and relates to a pharmaceutical composition comprising a compound of Formula (I) as well as methods of using such a compound for treatment of diseases, including cancers.

### BACKGROUND OF THE INVENTION

RAS, which is a small monomeric GTP-binding protein having a molecular weight of about 21 kDa, acts as a molecular on/off switch. RAS can bind to GTP by binding to proteins of a guanine nucleotide exchange factor (GEF) (e.g., SOS1), which forces the release of a bound nucleotide, and releasing GDP. When RAS binds to GTP, it becomes activated (turned on) and recruits and activates proteins necessary for the propagation of other receptors' signals, such as c-Raf and PI 3-kinase. RAS also possesses enzymatic activity with which it cleaves the terminal phosphate of the GTP nucleotide and converts it to GDP. The rate of conversion is usually slow, but can be dramatically sped up by a protein of the GTPase-activating protein (GAP) class, such as RasGAP. When GTP is converted into GDP, RAS is deactivated (turned off).

The commonly known members of the RAS subfamily include HRAS, KRAS, and NRAS. Of these, mutations of KRAS are observed in many malignant tumors: in 95% of pancreatic cancers, in 45% of colorectal cancers, and in 35% of lung cancers. The mutations often occur in the glycine residue at position 12; in pulmonary adenocarcinoma, in particular, the mutation in the glycine residue at position 12 occurs in about 90% of the total KRAS mutations Among such mutations, the most prevalent mutation (44%) has been reported to be a mutation into cysteine (Nature Reviews Drug Discovery, 13 (11), 828-51, 2014).

KRAS proteins having the G12C mutation have historically thought to exist in a constitutively active state (GTP-bound) in cancer cells. However, a recent study indicated that KRAS proteins having the G12C mutation have basal GTPase activity. K-Ras has a pocket structure to which a therapeutic agent can bind. Part of the pocket contains Switch 1 (residue 30 to 40) and Switch 2 (residue 60 to 76). Switch 1 has threonine-35 and Switch 2 has glycine-60, and these amino acids respectively form a hydrogen bond with the γ-phosphoric acid of GTP, which keeps Switch 1 and Switch 2 in an active form. These two regions will be released by hydrolysis of GTP and liberate phosphoric acid to form an inactive GDP form. When GTP bound to K-Ras is replaced with GDP, the three-dimensional conformation of the switch region containing these switches is changed. The change may relate to a bond between K-Ras and a target protein, such as c-Raf.

Actually, it was reported that ARS-853 binds to the cysteine residue of the G12C mutant of inactive KRAS (GDP), thus preventing conversion of inactive KRAS (GDP) to active KRAS (GTP), inhibiting downstream signaling, and inducing apoptosis in cancer cells with the KRAS G12C mutation (WO 2014/152588; Cancer Discov., 6 (3), 316-29, 2016). It has also been reported that ARS-1620 with a quinazoline backbone exerts antitumor action in tumor-bearing mice expressing the KRAS G12C mutation by improving metabolic stability in mice (WO 2015/054572; Cell, 172 (3), 578-89, 2018).

However, because of its mode of action, there is a possibility that the inhibitors which bind to the inactive form of KRAS G12C are not able to exert sufficient effect to KRAS G12C-positive cancer patients in whom the active form of KRAS protein (GTP) tends to be increased by activation of a KRAS upstream pathway or deactivation of GTPase activity in clinical settings. In fact, it has been reported that the inhibition of KRAS activity and the antiproliferative effect of ARS-853 are attenuated by EGFR activation in a KRAS G12C mutated cell line (Cancer Discov., 6 (3), 316-29, 2016; Science, 351 (6273), 604-8, 2016).

### SUMMARY OF THE DISCLOSURE

The present disclosure provides heterocyclic compounds which modulate mutant KRAS, HRAS, and/or NRAS proteins and may be valuable pharmaceutically active compounds for the treatment of cancer. In some embodiments the disclosed compounds selectively inhibit the KRAS G12C protein. The compounds of Formula (I):

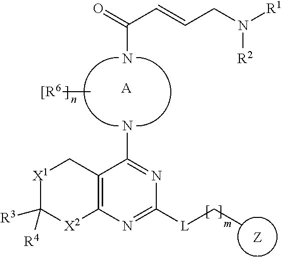

and their pharmaceutically acceptable salts, can modulate the activity of KRAS, HRAS and/or NRAS activity and thereby affect the signaling pathway which regulates cell growth, differentiation, and proliferation associated with oncological disorders. In certain embodiments, the compounds of Formula (I) can inhibit the KRAS G12C protein. The disclosure furthermore provides processes for preparing compounds of Formula (I), methods for using such compounds to treat oncological disorders, and pharmaceutical compositions which comprise compounds of Formula (I).

### DETAILED DESCRIPTION OF THE INVENTION

### Compounds of the Disclosure

In one embodiment, the present disclosure provides a compound having structural Formula (I), or a pharmaceutically acceptable salt thereof, as shown above, wherein: one of X¹ and X² is O and the other is C(R⁵);
R⁵ is H;
R³ is ring C³, wherein ring C³ is selected from the group consisting of
   (i) a 9- or 10-membered bicyclic aryl containing 0 to 3 ring atoms selected from the group consisting of N, O, and S;
   (ii) phenyl; and
   (iii) pyridyl;
   wherein ring C³ is unsubstituted or substituted by 1 to 4 R^{C3} substituents independently selected from the group consisting of halo, C₁-C₃ alkyl, C₁-C₃ fluoroalkyl, C₂-C₄ alkenyl, C₂-C₄ alkynyl, C₁-C₃ alkoxy, hydroxy, amino, and cyano;
R⁴ is H;
or alternatively, R³ and R⁴ together with the carbon atom to which they are attached form a group

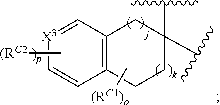

X³ is C(H) or N;
R^{C1} is independently selected from the group consisting of fluoro, C₁-C₃ alkyl, and C₁-C₃ fluoroalkyl;
R^{C2} is independently selected from the group consisting of halo, C₁-C₃ alkyl, C₁-C₃ fluoroalkyl, C₂-C₄ alkynyl, C₁-C₃ alkoxy, hydroxy, amino, and cyano;
subscript j is 0 or 1;
subscript k is 0, 1 or 2;
subscript o is 0, 1, 2, or 3;
subscript p is 0, 1, 2, 3, or 4;
A is a 5- to 8-membered monocyclic or 7- to 9-membered bicyclic saturated heterocyclic ring containing 0 to 1 additional heteroatoms selected from the group consisting of N, O, and S;
R¹ and R² are independently selected from the group consisting of: H;
   C₁-C₆ alkyl;
   C₁-C₆ fluoroalkyl; and
   a 3- to 8-membered mono- or bridged bicyclic saturated ring containing 0 to 2 heteroatoms selected from the group consisting of N, O, and S;
   wherein the 3- to 8-membered mono- or bridged bicyclic saturated ring is unsubstituted or substituted by 1 to 4 R^{1a} substituents independently selected from the group consisting of fluoro, C₁-C₃ alkyl, C₁-C₃ fluoroalkyl, C₁-C₃ alkoxy, hydroxy, and C₁-C₃ hydroxyalkyl;
   or alternatively, R¹ and R² together with the N atom to which they are attached form a 5- to 8-membered saturated heterocyclic ring having 0 to 1 additional heteroatoms selected from the group consisting of N, O, and S;
   wherein the 5- to 8-membered saturated heterocyclic ring is unsubstituted or substituted by 1 to 4 Rib substituents independently selected from the group consisting of halo, C₁-C₃ alkyl, C₁-C₃ fluoroalkyl, C₁-C₃ alkoxy, hydroxy, and C₁-C₃ hydroxyalkyl;
R⁶ is selected from the group consisting of halo, C₁-C₃ alkyl, C₁-C₃ fluoroalkyl, C₁-C₄ cyanoalkyl, and C₁-C₃ hydroxyalkyl;
Ring Z is selected from the group consisting of
   (i) a 3- to 10-membered mono- or bicyclic cycloalkyl;
   (ii) a 3- to 10-membered mono- or bicyclic heterocycloalkyl, wherein said heterocycloalkyl is saturated and contains 1 to 2 heteroatoms selected from the group consisting of N, S, and O; and
   (iii) a 3- to 8-membered spiroheterocycloalkyl, wherein said spiroheterocycloalkyl is saturated and contains 1 to 2 heteroatoms selected from the group consisting of N, S, and O;
   wherein Ring Z is unsubstituted or substituted by 1 to 4 R^{Z} substituents independently selected from the group consisting of fluoro, hydroxy, C₁-C₆ alkyl, C₁-C₃ alkoxy, C₁-C₃ hydroxyalkyl, C₁-C₃ alkylsulfinyl, C₁-C₃ alkylsulfonyl, C₁-C₃ fluoroalkyl, methoxy(C₁-C₃)alkyl, carboxy, (R^{a})₂NC(O)—, (R^{a})₂NC(O)(C₁-C₃) alkyl, (R^{a})₂N(C₁-C₃)alkyl, and (R^{a})₂NC(O)—O—(C₁-C₃) alkyl;
   each occurrence of R^{a} is independently H or C₁-C₃ alkyl;
   Ring Z is optionally substituted by 1-M-R^{ZC}, wherein
      M is —CH₂— or absent; and
      R^{ZC} is a 5- to 6-membered mono- or a 9- to 10-membered bicyclic saturated heterocycloalkyl which contains 1 to 3 heteroatoms selected from the group consisting of N, S, and O, wherein R^{ZC} is unsubstituted or substituted by 1-3 substituents independently selected from the group consisting of C₁-C₃ alkyl, C₁-C₃ alkylcarbonylalkyl, C₁-C₃ hydroxyalkyl, fluoro, cyano, (R^{a})₂N—, C₁-C₃ alkoxyalkyl, (R^{a})₂NC(O) (C₁-C₃) alkyl, and C₁-C₄ cyanoalkyl;
L is O or absent;
subscript m is 0, 1, or 2; and
subscript n is 0, 1, 2, or 3.

In another embodiment, the present disclosure provides a compound of Formula (I), wherein ring A is piperazine.

In another embodiment, the present disclosure provides a compound of Formula (I), wherein R⁶ is —CH₂CN and subscript n is 1.

In another embodiment, the present disclosure provides a compound of Formula (I), wherein the group

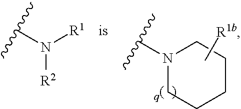

wherein R^{1b} is C₁-C₃ alkoxy, hydroxy, or C₁-C₃ hydroxyalkyl; and subscript q is 1, 2, or 3.

In another embodiment, the present disclosure provides a compound of Formula (I), wherein the group

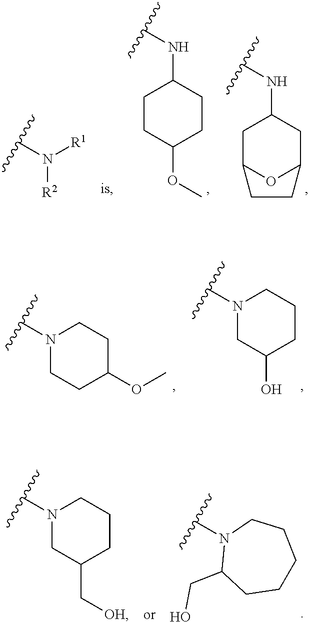

In another embodiment, the present disclosure provides a compound of Formula (I), wherein the moiety is

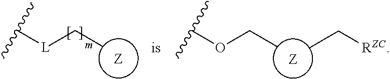

In another embodiment, the present disclosure provides a compound of Formula (I), wherein the moiety is:

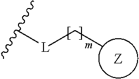

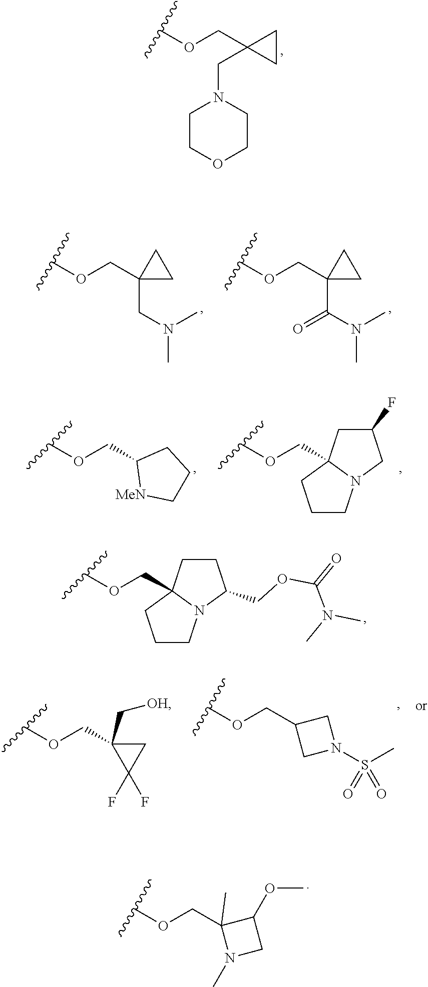

In another embodiment, the present disclosure provides a compound of Formula (I), wherein XI is O and X² is C(R⁵).

In another embodiment, the present disclosure provides a compound of Formula (I), wherein R³ is unsubstituted naphthyl or naphthyl substituted by 1 to 4 R^{C3} substituents; and R⁴ is H.

In another embodiment, the present disclosure provides a compound of Formula (I), wherein R³ is

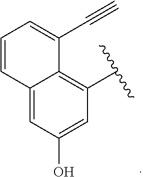 [SMILES: *c1cc(O)cc2cccc(C#C)c12]

In another embodiment, the present disclosure provides a compound of Formula (I), wherein R³ and R⁴, together with the carbon atom to which they are attached, form a group

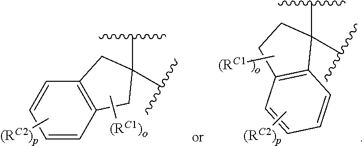

In another embodiment, the present disclosure provides a compound of Formula (I), wherein the compound of Formula (I) has the Formula (IA)

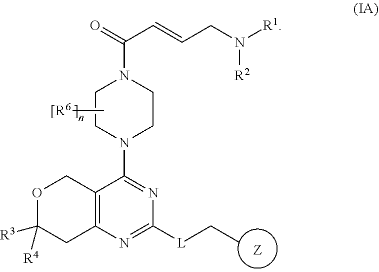

In another embodiment, the present disclosure provides a compound of Formula (I), wherein the group

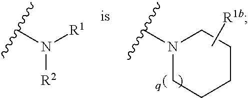

R^{1b} is C₁-C₃ alkoxy, hydroxy, or C₁-C₃ hydroxyalkyl;
subscript q is 1, 2, or 3;
R⁶ is —CH₂CN and subscript n is 1;
the moiety

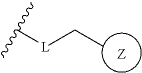

is:

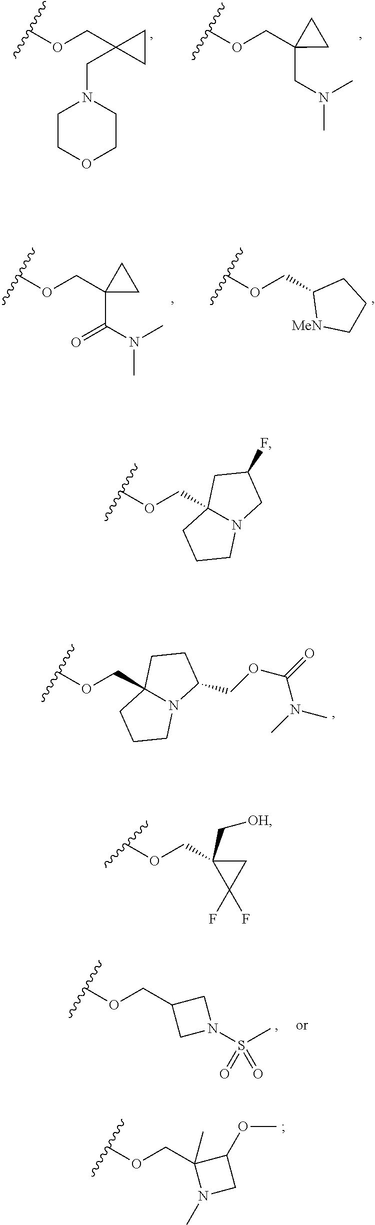

and
R³ is

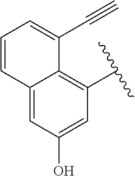 [SMILES: *c1cc(O)cc2cccc(C#C)c12]

and R⁴ is H.

In specific embodiments, the present disclosure provides for a compound selected from

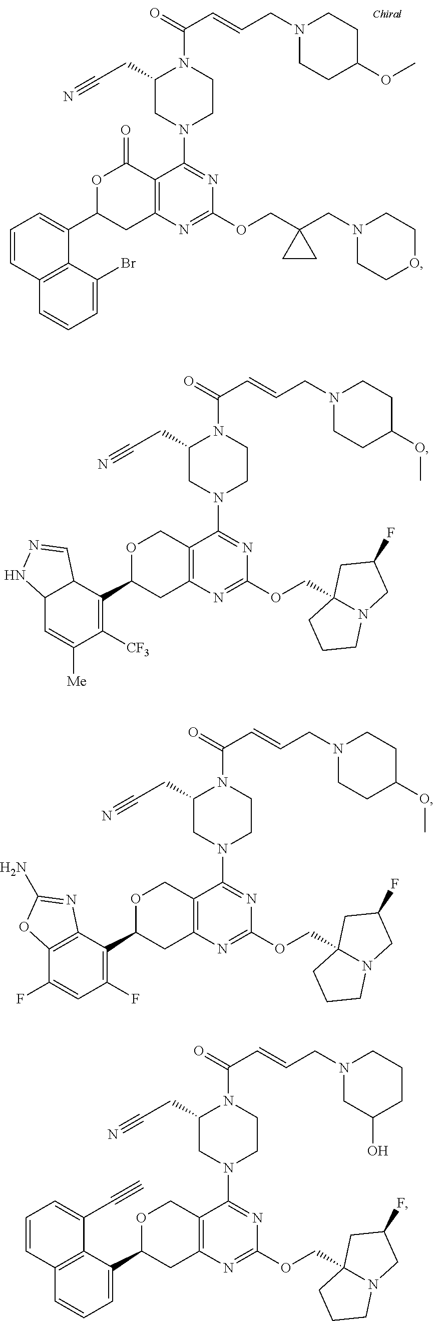 [SMILES: COC1CCN(C/C=C/C(=O)N2CCN(c3nc(OCC4(CN5CCOCC5)CC4)nc4c3C(=O)OC(c3cccc5cccc(Br)c35)C4)C[C@@H]2CC#N)CC1 ; COC1CCN(C/C=C/C(=O)N2CCN(c3nc(OC[C@@]45CCCN4C[C@H](F)C5)nc4c3CO[C@H](C3=C(C(F)(F)F)C(C)=CC5NN=CC35)C4)C[C@@H]2CC#N)CC1 ; COC1CCN(C/C=C/C(=O)N2CCN(c3nc(OC[C@@]45CCCN4C[C@H](F)C5)nc4c3CO[C@H](c3c(F)cc(F)c5oc(N)nc35)C4)C[C@@H]2CC#N)CC1 ; C#Cc1cccc2cccc([C@@H]3Cc4nc(OC[C@@]56CCCN5C[C@H](F)C6)nc(N5CCN(C(=O)/C=C/CN6CCCC(O)C6)[C@@H](CC#N)C5)c4CO3)c12]

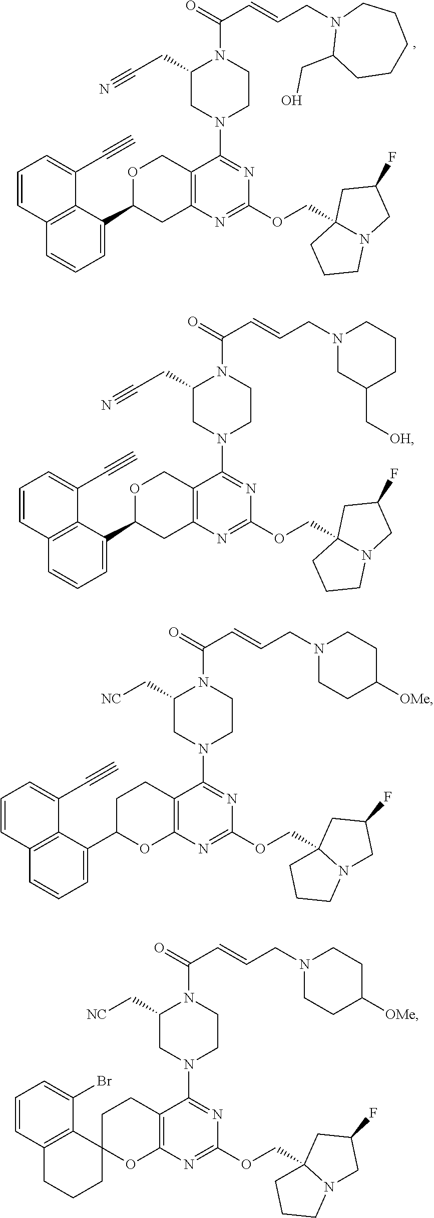

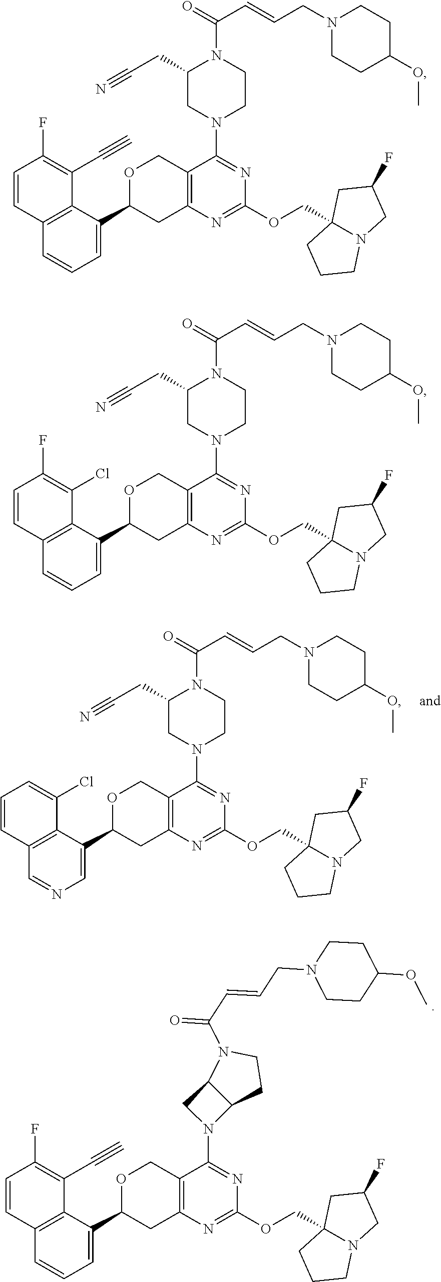

In specific embodiments, the present disclosure provides a compound as described in any one of Examples 1-26 as set forth below, or a pharmaceutically acceptable salt thereof.

The present disclosure includes the pharmaceutically acceptable salts of the compounds defined herein, including the pharmaceutically acceptable salts of all structural formulas, embodiments and classes defined herein.

### Definitions

Unless defined otherwise, all technical and scientific terms used herein have the same meaning as is commonly understood by one of skill in the art to which this invention belongs.

As used throughout this disclosure, “a compound of Formula (I)” is to be understood to include “a compound of Formula (I) or a pharmaceutically acceptable salt thereof”. Likewise “a compound of Formula (I)”, “compound(s) disclosed herein”, “compound(s) described herein”, “compound(s) of the disclosure”, etc., are used interchangeably and include both the compound, as well as a pharmaceutically acceptable salt thereof.

“Alkyl”, as well as other groups having the prefix “alk”, such as alkoxy, and the like, means carbon chains which may be linear or branched, or combinations thereof, containing the indicated number of carbon atoms. For instance, a C₁-C₆ alkyl means an alkyl group having one (i.e., methyl) up to 6 carbon atoms (i.e., hexyl). In particular embodiments, linear alkyl groups have 1-6 carbon atoms and branched alkyl groups have 3-7 carbon atoms. Examples of alkyl groups include methyl, ethyl, propyl, isopropyl, butyl, sec- and tert-butyl, pentyl, hexyl, heptyl, octyl, nonyl and the like.

“Alkoxy” and “alkyl-O—” are used interchangeably and refer to an alkyl group linked to oxygen, which is, in turn, attached to the parent moiety.

“Alkoxyalkyl” means an alkoxy group attached to an alkyl group in which the alkoxy and alkyl groups are as previously defined, and the bond to the parent moiety is through a carbon atom of the alkyl group. Non-limiting examples of suitable alkoxyalkyl groups include methoxyalkyl groups such as methoxymethyl and methoxyethyl.

“Alkylcarbonylalkyl” means a carbonyl group positioned between two alkyl groups, in which the alkyl groups are previously defined.

“Alkenyl” means an aliphatic hydrocarbon group containing at least one carbon-carbon double bond and which may be straight or branched. Branched means that one or more alkyl groups such as methyl, ethyl or propyl, are attached to a linear alkenyl chain. Non-limiting examples of alkenyl groups include ethenyl, propenyl, n-butenyl, 3-methylbut-2-enyl, and n-pentenyl.

“Alkynyl” means an aliphatic hydrocarbon group containing at least one carbon-carbon triple bond and which may be straight or branched. Non-limiting examples include ethynyl, propynyl, and butynyl.

“Aryl” means a monocyclic, bicyclic or tricyclic carbocyclic aromatic ring or ring system containing 5-14 carbon atoms, wherein at least one of the rings is aromatic. Non-limiting examples include phenyl and naphthyl.

“Alkylsulfonyl” means an alkyl-SO₂ group in which the alkyl group is previously defined, i.e., —S(O)₂-alkyl. The bond to the parent moiety is through the sulfur atom of the sulfonyl moiety. Non-limiting examples of suitable alkylsulfonyl groups include methylsulfonyl and ethylsulfonyl.

“Alkylsulfinyl” means an alkyl-S(O) group in which the alkyl group is previously defined, i.e., —S(O)-alkyl. The bond to the parent moiety is through the sulfur atom of the sulfinyl moiety. Non-limiting examples of suitable alkylsulfinyl groups include methylsulfinyl and ethylsulfinyl.

“Bicyclic ring system” refers to two joined rings. The rings may be fused, i.e., share two adjacent atoms, “spirocyclic”, i.e., share only a single atom, or “bridged”, i.e., share three or more atoms with two bridgehead atoms being connected by a bridge containing at least one atom. Likewise the bicyclic rings may be aryl rings, heterocyclic rings, cycloalkyl rings, etc.

“Cyanoalkyl” means an -alkyl-CN group in which the alkyl is as previously defined. The bond to the parent moiety is through a carbon atom of the alkyl group. Non-limiting examples of suitable cyanoalkyl groups include cyanomethyl and 3-cyanopropyl.

“Cycloalkyl” means a saturated cyclic hydrocarbon radical. In particular embodiments, the cycloalkyl group has 3-12 carbon atoms and may form 2-3 carbocyclic rings that are fused. Examples of cycloalkyl include cyclopropyl, cyclobutyl, cyclopentyl, cyclohexyl, cycloheptyl, adamantyl, and the like.

“Fluoroalkyl” includes mono-substituted as well as multiple fluoro-substituted alkyl groups, up to perfluoro substituted alkyl. For example, fluoromethyl, 1,1-difluoroethyl, trifluoromethyl or 1,1,1,2,2-pentafluorobutyl are included.

“Halogen” or “halo”, unless otherwise indicated, includes fluorine (fluoro), chlorine (chloro), bromine (bromo) and iodine (iodo). In one embodiment, halo is fluoro (—F) or chloro (—Cl).

“Heterocycloalkyl” or “heterocyclic ring” or “heterocycle” means a non-aromatic monocyclic, bicyclic, tricyclic or bridged ring system comprising about 3 to about 10 ring atoms, preferably about 5 to about 10 ring atoms, in which one or more of the atoms in the ring system is an element other than carbon, for example, nitrogen, oxygen, phosphorus or sulfur, alone or in combination. There are no adjacent oxygen and/or sulfur atoms present in the ring system. In some embodiments, heterocycloalkyls contain about 5 to about 6 ring atoms. The prefix aza, oxa, phospha or thia before the heterocyclyl root name means that at least a nitrogen, oxygen, phosphorus or sulfur atom respectively is present as a ring atom. In some embodiments, the nitrogen or sulfur atom of the heterocycloalkyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, phosphorinane, phosphinane, 1-oxophosphinan-1-ium and the like. “Spiroheterocycloalkyl” refers to a fused ring system in which the rings share only a single atom and at least one of the rings is a heterocycloalkyl.

“Hydroxyalkyl” means a HO-alkyl- group in which alkyl is as previously defined. The bond to the parent moiety is through a carbon atom of the alkyl group. Preferred hydroxyalkyls contain lower alkyl. Non-limiting examples of suitable hydroxyalkyl groups include hydroxymethyl and 2-hydroxyethyl.

When any variable (e.g., R⁶) occurs more than one time in any constituent or in Formula (I) or other generic formulas herein, its definition on each occurrence is independent of its definition at every other occurrence. Combinations of substituents and/or variables are permissible only if such combinations result in stable compounds. In choosing compounds of the present disclosure, one of ordinary skill in the art will recognize that the various substituents, e.g., R⁶, are to be chosen in conformity with well-known principles of chemical structure connectivity and stability. Unless expressly stated to the contrary, substitution by a named substituent is permitted on any atom in a ring (e.g., aryl, a heteroaryl ring, or a saturated heteroaryl ring) provided such ring substitution is chemically allowed and results in a stable compound. A “stable” compound is a compound which can be prepared and isolated and whose structure and properties remain or can be caused to remain essentially unchanged for a period of time sufficient to allow use of the compound for the purposes described herein (e.g., therapeutic or prophylactic administration to a subject).

The term “substituted” shall be deemed to include multiple degrees of substitution by a named substituent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

Unless expressly depicted or described otherwise, variables depicted in a structural formula with a “floating” bond, such as R⁶, are permitted on any available carbon atom in the ring to which the variable is attached. When a moiety is noted as being “optionally substituted” in Formula (I) or any embodiment thereof, it means that Formula (I) or the embodiment thereof encompasses compounds that contain the noted substituent (or substituents) on the moiety and also compounds that do not contain the noted substituent (or substituents) on the moiety.

The wavy line [symbol] , as used herein, indicates a point of attachment to the rest of the compound.

The compounds of Formula (I) may contain one or more asymmetric centers and can thus occur as racemates and racemic mixtures, single enantiomers, diastereoisomeric mixtures and individual diastereoisomers. Centers of asymmetry that are present in the compounds of Formula (I) can all independently of one another have S configuration or R configuration. The compounds of Formula (I) include all possible enantiomers and diastereomers and mixtures of two or more stereoisomers, for example, mixtures of enantiomers and/or diastereomers, in all ratios. Thus, enantiomers are a subject of the disclosure in enantiomerically pure form, both as levorotatory and as dextrorotatory antipodes, in the form of racemates and in the form of mixtures of the two enantiomers in all ratios. In the case of a cis/trans isomerism, the disclosure includes both the cis form and the trans form as well as mixtures of these forms in all ratios. The present disclosure is meant to comprehend all such stereoisomeric forms of the compounds of Formula (I). Where a structural formula or chemical name specifies a particular configuration at a stereocenter, the enantiomer or stereoisomer of the compound resulting from that specified stereocenter is intended. Where a structural formula of the compounds of Formula (I) indicates a straight line at a chiral center, the structural formula includes both the S and R stereoisomers associated with the chiral center and mixtures thereof.

The compounds of Formula (I) may be separated into their individual diastereoisomers by, for example, fractional crystallization from a suitable solvent, for example, methanol or ethyl acetate or a mixture thereof, or via chiral chromatography using an optically active stationary phase. Absolute stereochemistry may be determined by X-ray crystallography of crystalline products or crystalline intermediates which are derivatized, if necessary, with a reagent containing an asymmetric center of known absolute configuration. Vibrational circular dichroism (VCD) may also be used to determine the absolute stereochemistry. Alternatively, any stereoisomer or isomers of the compounds of Formula (I) may be obtained by stereospecific synthesis using optically pure starting materials or reagents of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereoisomeric mixture, followed by separation of the individual diastereoisomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

The compounds of Formula (I) which contain olefinic double bonds, unless specified otherwise, they are meant to include both E and Z geometric isomers.

Some of the compounds described herein may exist as tautomers which have different points of attachment of hydrogen accompanied by one or more double bond shifts. For example, a ketone and its enol form are keto-enol tautomers. The individual tautomers as well as mixtures thereof are encompassed by the compounds of Formula (I).

Some of the compounds of Formula (I) described herein may exist as atropisomers when the rotational energy barrier around a single bond is sufficiently high to prevent free rotation at a given temperature, thus allowing isolation of individual conformers with distinct properties. The individual atropisomers as well as mixtures thereof are encompassed with compounds of Formula (I) of the present disclosure. When resolved, individual atropisomers can be designated by established conventions such as those specified by the International Union of Pure Applied Chemistry (IUPAC) 2013 Recommendations.

In the compounds of Formula (I), the atoms may exhibit their natural isotopic abundances, or one or more of the atoms may be artificially enriched in a particular isotope having the same atomic number, but an atomic mass or mass number different from the atomic mass or mass number predominantly found in nature. The present disclosure as described and claimed herein is meant to include all suitable isotopic variations of the compounds of Formula (I) and embodiments thereof. For example, different isotopic forms of hydrogen (H) include protium (¹H) and deuterium (²H, also denoted herein as D). Protium is the predominant hydrogen isotope found in nature. Enriching for deuterium may afford certain therapeutic advantages, such as increasing in vivo half-life or reducing dosage requirements or may provide a compound useful as a standard for characterization of biological samples. Isotopically-enriched compounds can be prepared without undue experimentation by conventional techniques well known to those skilled in the art or by processes analogous to those described in the Schemes and Examples herein using appropriate isotopically-enriched reagents and/or intermediates.

The term “pharmaceutically acceptable salts” refers to salts prepared from pharmaceutically acceptable non-toxic bases or acids. When a compound of Formula (I) is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Preferred are the ammonium, calcium, magnesium, potassium and sodium salts. Salts prepared from pharmaceutically acceptable organic non-toxic bases include salts of primary, secondary, and tertiary amines derived from both naturally occurring and synthetic sources. Pharmaceutically acceptable organic non-toxic bases from which salts can be formed include, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, dicyclohexylamine, lysine, methylglucamine, morpholine, piperazine, piperidine, polyamine resins, procaine, purines, theobromine, triethylamine, trimethylamine, tripropylamine, tromethamine and the like.

When a compound of Formula (I) is basic, its corresponding salt can be conveniently prepared from pharmaceutically acceptable non-toxic inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, hydrochloric, maleic, phosphoric, sulfuric, and tartaric acids. If a compound of Formula (I) simultaneously contain acidic and basic groups in the molecule, the disclosure also includes, in addition to the salt forms mentioned, inner salts or betaines (zwitterions). Salts can be obtained from the compounds of Formula (I) by customary methods which are known to the person skilled in the art, for example, by combination with an organic or inorganic acid or base in a solvent or dispersant, or by anion exchange or cation exchange from other salts. The present disclosure also includes all salts of the compounds of Formula (I) which, owing to low physiological compatibility, are not directly suitable for use in pharmaceuticals but which can be used, for example, as intermediates for chemical reactions or for the preparation of pharmaceutically acceptable salts.

Furthermore, the compounds of Formula (I) may exist in amorphous form and/or one or more crystalline forms, and as such all amorphous and crystalline forms and mixtures thereof of the compounds of Formula (I), including the Examples, are intended to be included within the scope of the present disclosure. In addition, some of the compounds of Formula (I) may form solvates with water (i.e., a hydrate) or common organic solvents such as but not limited to ethyl acetate. Such solvates and hydrates, particularly the pharmaceutically acceptable solvates and hydrates, of the instant compounds are likewise encompassed within the scope of this disclosure, along with un-solvated and anhydrous forms.

Any pharmaceutically acceptable pro-drug modification of a compound of Formula (I) which results in conversion in vivo to a compound within the scope of this disclosure is also within the scope of this disclosure.

The terms “therapeutically effective (or efficacious) amount” and similar descriptions such as “an amount efficacious for treatment” or “an effective dose” are intended to mean that amount of a compound of Formula (I) that will elicit the biological or medical response of a tissue, a system, animal or human that is being sought by a researcher, veterinarian, medical doctor or other clinician. In a preferred embodiment, the term “therapeutically effective amount” means an amount of a compound of Formula (I) that alleviates at least one clinical symptom in a human patient. The terms “prophylactically effective (or efficacious) amount” and similar descriptions such as “an amount efficacious for prevention” are intended to mean that amount of a compound of Formula (I) that will prevent or reduce the risk of occurrence of the biological or medical event that is sought to be prevented in a tissue, a system, animal or human by a researcher, veterinarian, medical doctor or other clinician.

### Dosages of the Compounds of Formula (I)

The dosage regimen utilizing a compound of Formula (I) is selected in accordance with a variety of factors including type, species, age, weight, sex and medical condition of the patient; the severity of the condition to be treated; the potency of the compound chosen to be administered; the route of administration; and the renal and hepatic function of the patient. A consideration of these factors is well within the purview of the ordinarily skilled clinician for the purpose of determining the therapeutically effective or prophylactically effective dosage amount needed to prevent, counter, or arrest the progress of the condition. It is understood that a specific daily dosage amount can simultaneously be both a therapeutically effective amount, e.g., for treatment of an oncological condition, and a prophylactically effective amount, e.g., for prevention of an oncological condition.

While individual needs vary, determination of optimal ranges of effective amounts of the compounds of Formula (I) is within the skill of the art. For administration to a human in, for example, the curative or prophylactic treatment of the conditions and disorders identified herein, the typical dosages of the compounds of Formula (I) can be about 0.05 mg/kg/day to about 50 mg/kg/day, or at least 0.05 mg/kg, or at least 0.08 mg/kg, or at least 0.1 mg/kg, or at least 0.2 mg/kg, or at least 0.3 mg/kg, or at least 0.4 mg/kg, or at least 0.5 mg/kg, and any amount therebetween, to about 50 mg/kg or less, or about 40 mg/kg or less, or about 30 mg/kg or less, or about 20 mg/kg or less, or about 10 mg/kg or less and any amount therebetween, which can be, for example, about 2.5 mg/day (0.5 mg/kg×5 kg) to about 5000 mg/day (50 mg/kg×100 kg). For example, dosages of the compounds can be about 0.1 mg/kg/day to about 50 mg/kg/day, or about 0.05 mg/kg/day to about 10 mg/kg/day, or about 0.05 mg/kg/day to about 5 mg/kg/day, or about 0.05 mg/kg/day to about 3 mg/kg/day, or about 0.07 mg/kg/day to about 3 mg/kg/day, or about 0.09 mg/kg/day to about 3 mg/kg/day, or about 0.05 mg/kg/day to about 0.1 mg/kg/day, or about 0.1 mg/kg/day to about 1 mg/kg/day, or about 1 mg/kg/day to about 10 mg/kg/day, or about 1 mg/kg/day to about 5 mg/kg/day, or about 1 mg/kg/day to about 3 mg/kg/day, or about 3 mg/day to about 500 mg/day, or about 5 mg/day to about 250 mg/day, or about 10 mg/day to about 100 mg/day, or about 3 mg/day to about 10 mg/day, or about 100 mg/day to about 250 mg/day. Such doses may be administered in a single dose or may be divided into multiple doses.

### Pharmaceutical Compositions

The compounds of Formula (I) and their pharmaceutically acceptable salts can be administered to animals, preferably to mammals, and in particular to humans, as pharmaceuticals by themselves, in mixtures with one another or in the form of pharmaceutical compositions. The term “subject” or “patient” includes animals, preferably mammals and especially humans, who use the instant active agents for the prevention or treatment of a medical condition. Administering of the drug to the subject includes both self-administration and administration to the patient by another person. The subject may be in need of, or desire, treatment for an existing disease or medical condition, or may be in need of or desire prophylactic treatment to prevent or reduce the risk of occurrence of said disease or medical condition. As used herein, a subject “in need” of treatment of an existing condition or of prophylactic treatment encompasses both a determination of need by a medical professional as well as the desire of a patient for such treatment.

The present disclosure therefore also provides the compounds of Formula (I) and their pharmaceutically acceptable salts for use as pharmaceuticals, their use for modulating the activity of mutant KRAS, HRAS and/or NRAS proteins and in particular their use in the therapy and prophylaxis of the below-mentioned diseases or disorders as well as their use for preparing medicaments for these purposes. In certain embodiments, the compounds of Formula (I) and their pharmaceutically acceptable salts inhibit the KRAS G12C protein.

Furthermore, the present disclosure provides pharmaceutical compositions which comprise as active component an effective dose of at least one compound of Formula (I) and/or a pharmaceutically acceptable salt thereof and a customary pharmaceutically acceptable carrier, i.e., one or more pharmaceutically acceptable carrier substances and/or additives.

Thus, the present disclosure provides, for example, said compound and its pharmaceutically acceptable salts for use as pharmaceutical compositions which comprise as active component an effective dose of at least one compound of Formula (I) and/or a pharmaceutically acceptable salt thereof and a customary pharmaceutically acceptable carrier, and the uses of said compound and/or a pharmaceutically acceptable salt thereof in the therapy or prophylaxis of the below-mentioned diseases or disorders, e.g., cancer, as well as their use for preparing medicaments for these purposes.

The pharmaceutical compositions according to the disclosure can be administered orally, for example, in the form of pills, tablets, lacquered tablets, sugar-coated tablets, granules, hard and soft gelatin capsules, aqueous, alcoholic or oily solutions, syrups, emulsions or suspensions, or rectally, for example, in the form of suppositories. Administration can also be carried out parenterally, for example subcutaneously, intramuscularly or intravenously in the form of solutions for injection or infusion.

Other suitable administration forms are, for example, percutaneous or topical administration, for example, in the form of ointments, tinctures, sprays or transdermal therapeutic systems, or, for example, microcapsules, implants or rods. The preferred administration form depends, for example, on the disease to be treated and on its severity.

The amount of active compound of a compound described herein and/or its pharmaceutically acceptable salts in the pharmaceutical composition normally is from 0.01 to 200 mg, or from 0.1 to 200 mg, or from 1 to 200 mg, per dose, but depending on the type of the pharmaceutical composition, it can also be higher. In some embodiments, the amount of active compound of a compound of Formula (I) and/or its pharmaceutically acceptable salts in the pharmaceutical composition is from 0.01 to 10 mg per dose. The pharmaceutical compositions usually comprise 0.5 to 90 percent by weight of at least one compound of Formula (I) and/or its pharmaceutically acceptable salts. The preparation of the pharmaceutical compositions can be carried out in a manner known per se. For this purpose, one or more compounds of Formula (I) and/or their pharmaceutically acceptable salts, together with one or more solid or liquid pharmaceutical carrier substances and/or additives (or auxiliary substances) and, if desired, in combination with other pharmaceutically active compounds having therapeutic or prophylactic action, are brought into a suitable administration form or dosage form which can then be used as a pharmaceutical in human or veterinary medicine.

For the production of pills, tablets, sugar-coated tablets and hard gelatin capsules, it is possible to use, for example, lactose, starch, for example, maize starch, or starch derivatives, talc, stearic acid or its salts, etc. Carriers for soft gelatin capsules and suppositories are, for example, fats, waxes, semisolid and liquid polyols, natural or hardened oils, etc. Suitable carriers for the preparation of solutions, for example, of solutions for injection, or of emulsions or syrups are, for example, water, physiologically acceptable sodium chloride solution, alcohols such as ethanol, glycerol, polyols, sucrose, invert sugar, glucose, mannitol, vegetable oils, etc. It is also possible to lyophilize the compounds of Formula (I) and their pharmaceutically acceptable salts and to use the resulting lyophilisates, for example, for preparing preparations for injection or infusion. Suitable carriers for microcapsules, implants or rods are, for example, copolymers of glycolic acid and lactic acid.

Besides the active compounds and carriers, the pharmaceutical compositions can also contain customary additives, for example, fillers, disintegrants, binders, lubricants, wetting agents, stabilizers, emulsifiers, dispersants, preservatives, sweeteners, colorants, flavorings, aromatizers, thickeners, diluents, buffer substances, solvents, solubilizers, agents for achieving a depot effect, salts for altering the osmotic pressure, coating agents and/or antioxidants.

### Methods of Using the Compounds of Formula (I)

The present application provides a method of inhibiting RAS-mediated cell signaling comprising contacting a cell with a compound of Formula (I) or a pharmaceutically acceptable salt thereof. Inhibition of RAS-mediated signal transduction can be assessed and demonstrated by a wide variety of ways known in the art. Non-limiting examples include (a) a decrease in GTPase activity of RAS; (b) a decrease in GTP binding affinity or an increase in GDP binding affinity; (c) an increase in K_{off} of GTP or a decrease in K_{off} of GDP; (d) a decrease in the levels of signaling transduction molecules downstream in the RAS pathway, such as a decrease in pMEK, pERK, or pAKT levels; and/or (e) a decrease in binding of RAS complex to downstream signaling molecules including but not limited to Raf. Kits and commercially available assays can be utilized for determining one or more of the above.

The present application also provides methods of using the compounds of Formula (I) (or their pharmaceutically acceptable salts) or pharmaceutical compositions containing such compounds to treat disease conditions, including but not limited to, conditions implicated by mutant KRAS, HRAS and/or NRAS proteins (e.g., cancer), and in some embodiments the KRAS G12C mutant.

In some embodiments, a method for treatment of cancer is provided, the method comprising administering a therapeutically effective amount a compound of Formula (I) (or a pharmaceutically acceptable salt thereof) or any of the foregoing pharmaceutical compositions comprising such a compound to a subject in need of such treatment. In some embodiments, the cancer is mediated by a KRAS, HRAS or NRAS mutation, e.g., the KRAS G12C mutation. In various embodiments, the cancer is pancreatic cancer, colorectal cancer or lung cancer. In some embodiments, the cancer is gall bladder cancer, thyroid cancer, or bile duct cancer.

In some embodiments the present disclosure provides a method of treating a disorder in a subject in need thereof, wherein said method comprises determining if the subject has a KRAS, HRAS or NRAS mutation (e.g., KRAS G12C mutation) and if the subject is determined to have the KRAS, HRAS or NRAS mutation, then administering to the subject a therapeutically effective amount of a compound of Formula (I) or a pharmaceutically acceptable salt thereof.

The disclosed compounds inhibit anchorage-independent cell growth and therefore have the potential to inhibit tumor metastasis. Accordingly, another embodiment of the present disclosure provides a method for inhibiting tumor metastasis, the method comprising administering an effective amount a compound of Formula (I).

KRAS, HRAS or NRAS mutations have also been identified in hematological malignancies (e.g., cancers that affect blood, bone marrow and/or lymph nodes). Accordingly, certain embodiments are directed to administration of the compounds of Formula (I) (e.g., in the form of a pharmaceutical composition) to a subject in need of treatment of a hematological malignancy. Such malignancies include, but are not limited to leukemias and lymphomas. For example, the presently disclosed compounds can be used for treatment of diseases such as acute lymphoblastic leukemia (ALL), acute myelogenous leukemia (AML), chronic lymphocytic leukemia (CLL), small lymphocytic lymphoma (SLL), chronic myelogenous leukemia (CML), acute monocytic leukemia (AMoL) and/or other leukemias. In other embodiments, the compounds are useful for treatment of lymphomas such as Hodgkin's lymphoma or non-Hodgkin's lymphoma. In various embodiments, the compounds are useful for treatment of plasma cell malignancies such as multiple myeloma, mantle cell lymphoma, and Waldenstrom's macroglubunemia.

Determining whether a tumor or cancer comprises a KRAS, HRAS or NRAS mutation (e.g., the KRAS G12C mutation) can be undertaken by assessing the nucleotide sequence encoding the KRAS, HRAS or NRAS protein, by assessing the amino acid sequence of the KRAS, HRAS or NRAS protein, or by assessing the characteristics of a putative KRAS, HRAS or NRAS mutant protein. The sequences of wild-type human KRAS, HRAS or NRAS are known in the art.

Methods for detecting a mutation in a KRAS, HRAS or NRAS nucleotide sequence are also known by those of skill in the art. These methods include, but are not limited to, polymerase chain reaction-restriction fragment length polymorphism (PCR-RFLP) assays, polymerase chain reaction-single strand conformation polymorphism (PCR-SSCP) assays, real-time PCR assays, PCR sequencing, mutant allele-specific PCR amplification (MASA) assays, direct sequencing, primer extension reactions, electrophoresis, oligonucleotide ligation assays, hybridization assays, TaqMan assays, SNP genotyping assays, high resolution melting assays and microarray analyses. In some embodiments, samples are evaluated for KRAS, HRAS or NRAS mutations (e.g., the KRAS G12C mutation) by real-time PCR. In real-time PCR, fluorescent probes specific for the KRAS, HRAS or NRAS mutation are used. When a mutation is present, the probe binds and fluorescence is detected. In some embodiments, the KRAS, HRAS or NRAS mutation is identified using a direct sequencing method of specific regions (e.g., exon 2 and/or exon 3) in the KRAS, HRAS or NRAS gene.

Methods for detecting a mutation in a KRAS, HRAS or NRAS protein (e.g., the KRAS G12C mutation) are known by those of skill in the art. These methods include, but are not limited to, detection of a KRAS, HRAS or NRAS mutant using a binding agent (e.g., an antibody) specific for the mutant protein, protein electrophoresis and Western blotting, and direct peptide sequencing.

A number of tissue samples can be assessed for determining whether a tumor or cancer comprises a KRAS, HRAS or NRAS mutation (e.g., the KRAS G12C mutation). In some embodiments, the sample is taken from a subject having a tumor or cancer. In some embodiments, the sample is a fresh tumor/cancer sample. In some embodiments, the sample is a frozen tumor/cancer sample. In some embodiments, the sample is a formalin-fixed paraffin-embedded sample. In some embodiments, the sample is a circulating tumor cell (CTC) sample. In some embodiments, the sample is processed to a cell lysate. In some embodiments, the sample is processed to DNA or RNA.

The present application also provides a method of treating a hyperproliferative disorder comprising administering a therapeutically effective amount of a compound of Formula (I), or a pharmaceutically acceptable salt thereof to a subject in need thereof. In some embodiments, said method relates to the treatment of a subject who suffers from a cancer such as acute myeloid leukemia, cancer in adolescents, adrenocortical carcinoma childhood, AIDS-related cancers (e.g., lymphoma and Kaposi's Sarcoma), anal cancer, appendix cancer, astrocytomas, atypical teratoid, basal cell carcinoma, bile duct cancer, bladder cancer, bone cancer, brain stem glioma, brain tumor, breast cancer, bronchial tumors, Burkitt lymphoma, carcinoid tumor, atypical teratoid, embryonal tumors, germ cell tumor, primary lymphoma, cervical cancer, childhood cancers, chordoma, cardiac tumors, chronic lymphocytic leukemia (CLL), chronic myelogenous leukemia (CML), chronic myleoproliferative disorders, colon cancer, colorectal cancer, craniopharyngioma, cutaneous T-cell lymphoma, extrahepatic ductal carcinoma in situ (DCIS), embryonal tumors, CNS cancer, endometrial cancer, ependymoma, esophageal cancer, esthesioneuroblastoma, Ewing sarcoma, extracranial germ cell tumor, extragonadal germ cell tumor, eye cancer, fibrous histiocytoma of bone, gall bladder cancer, gastric cancer, gastrointestinal carcinoid tumor, gastrointestinal stromal tumors (GIST), germ cell tumor, gestational trophoblastic tumor, hairy cell leukemia, head and neck cancer, heart cancer, liver cancer, Hodgkin's lymphoma, hypopharyngeal cancer, intraocular melanoma, islet cell tumors, pancreatic neuroendocrine tumors, kidney cancer, laryngeal cancer, lip and oral cavity cancer, liver cancer, lobular carcinoma in situ (LCIS), lung cancer, lymphoma, metastatic squamous neck cancer with occult primary, midline tract carcinoma, mouth cancer; multiple endocrine neoplasia syndromes, multiple myeloma/plasma cell neoplasm, mycosis fungoides, myelodysplasia syndromes, myelodysplastic/myeloproliferative neoplasms, multiple myeloma, Merkel cell carcinoma, malignant mesothelioma, malignant fibrous histiocytoma of bone and osteosarcoma, nasal cavity and paranasal sinus cancer, nasopharyngeal cancer, neuroblastoma, non-Hodgkin's lymphoma, non-small cell lung cancer (NSCLC), oral cancer, lip and oral cavity cancer, oropharyngeal cancer, ovarian cancer, pancreatic cancer, papillomatosis, paraganglioma, paranasal sinus and nasal cavity cancer, parathyroid cancer, penile cancer, pharyngeal cancer, pleuropulmonary blastoma, primary central nervous system (CNS) lymphoma, prostate cancer, rectal cancer, transitional cell cancer, retinoblastoma, rhabdomyosarcoma, salivary gland cancer, skin cancer, stomach (gastric) cancer, small cell lung cancer; small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, or viral-induced cancer. In some embodiments, said method relates to the treatment of a non-cancerous hyperproliferative disorder such as benign hyperplasia of the skin (e.g., psoriasis), restenosis, or prostate (e.g., benign prostatic hypertrophy (BPH)).

In some embodiments, the methods for treatment are directed to treating lung cancers, and the methods comprise administering a therapeutically effective amount of the compounds of Formula (I) (or pharmaceutical composition comprising such compounds) to a subject in need thereof. In certain embodiments, the lung cancer is a non-small cell lung carcinoma (NSCLC), for example, adenocarcinoma, squamous-cell lung carcinoma or large-cell lung carcinoma. In some embodiments, the lung cancer is a small cell lung carcinoma. Other lung cancers which the compounds of Formula (I) may provide therapeutic benefit for include, but are not limited to, glandular tumors, carcinoid tumors and undifferentiated carcinomas.

The present disclosure also provides methods of modulating a mutant KRAS, HRAS or NRAS protein activity (e.g., activity resulting from the KRAS G12C mutation) by contacting the protein with an effective amount of a compound of Formula (I). Modulation can be inhibiting or activating protein activity. In some embodiments, the present disclosure provides methods of inhibiting protein activity by contacting the mutant KRAS, HRAS or NRAS protein (e.g., KRAS G12C mutant) with an effective amount of a compound of Formula (I) in solution. In some embodiments, the present disclosure provides methods of inhibiting the mutant KRAS, HRAS or NRAS protein activity by contacting a cell, tissue, or organ that expresses the protein of interest. In some embodiments, the disclosure provides methods of inhibiting protein activity in subjects including, but not limited to, rodents and mammals (e.g., humans) by administering into the subjects an effective amount of a compound of Formula (I).

### Combination Therapies

One or more additional pharmacologically active agents may be administered in combination with a compound of Formula (I) (or a pharmaceutically acceptable salt thereof). An additional active agent (or agents) is intended to mean a pharmaceutically active agent (or agents) that is active in the body, including pro-drugs that convert to pharmaceutically active form after administration, which are different from the compound of Formula (I). The additional active agents also include free-acid, free-base and pharmaceutically acceptable salts of said additional active agents. Generally, any suitable additional active agent or agents, including chemotherapeutic agents or therapeutic antibodies, may be used in any combination with the compound of Formula (I) in a single dosage formulation (e.g., a fixed dose drug combination), or in one or more separate dosage formulations which allows for concurrent or sequential administration of the active agents (co-administration of the separate active agents) to subjects. In addition, the compounds of Formula (I) (or pharmaceutically acceptable salts thereof) can be administered in combination with radiation therapy, hormone therapy, surgery or immunotherapy.

The present application also provides methods for combination therapies in which the additional active agent is known to modulate other pathways, or other components of the same pathway, or even overlapping sets of target enzymes which are used in combination with a compound of Formula (I), or a pharmaceutically acceptable salt thereof. In one embodiment, such therapy includes, but is not limited to, the combination of one or more compounds of Formula (I) with chemotherapeutic agents, immunotherapeutic agents, hormonal and anti-hormonal agents, targeted therapy agents, and anti-angiogenesis agents, to provide a synergistic or additive therapeutic effect. In another embodiment, such therapy includes radiation treatment to provide a synergistic or additive therapeutic effect.

Examples of additional active agents (i.e., additional anti-cancer agents) include chemotherapeutic agents (e.g., cytotoxic agents), immunotherapeutic agents, hormonal and anti-hormonal agents, targeted therapy agents, and anti-angiogenesis agents. Many anti-cancer agents can be classified within one or more of these groups. While certain anti-cancer agents have been categorized within a specific group(s) or subgroup(s) herein, many of these agents can also be listed within one or more other group(s) or subgroup(s), as would be presently understood in the art. It is to be understood that the classification herein of a particular agent into a particular group is not intended to be limiting. Many anti-cancer agents are presently known in the art and can be used in combination with the compounds of the present disclosure.

Further, an agent can be an agonist, antagonist, allosteric modulator, toxin or, more generally, may act to inhibit or stimulate its target (e.g., receptor or enzyme activation or inhibition). For example, suitable for use are one or more agents (e.g., antibodies, antigen binding regions, or soluble receptors) that specifically bind and inhibit the activity of growth factors, such as antagonists of hepatocyte growth factor (HGF, also known as Scatter Factor), and antibodies or antigen binding regions that specifically bind its receptor “c-met”.

In an embodiment, the additional anti-cancer agent is a chemotherapeutic agent, an immunotherapeutic agent, a hormonal agent, an anti-hormonal agent, a targeted therapy agent, or an anti-angiogenesis agent (or angiogenesis inhibitor). In an embodiment, the additional anti-cancer agent is selected from the group consisting of a chemotherapeutic agent, a mitotic inhibitor, a plant alkaloid, an alkylating agent, an anti-metabolite, a platinum analog, an enzyme, a topoisomerase inhibitor, a retinoid, an aziridine, an antibiotic, a hormonal agent, an anti-hormonal agent, an anti-estrogen, an anti-androgen, an anti-adrenal, an androgen, a targeted therapy agent, an immunotherapeutic agent, a biological response modifier, a cytokine inhibitor, a tumor vaccine, a monoclonal antibody, an immune checkpoint inhibitor, an anti-PD-1 agent, an anti-PD-L1 agent, a colony-stimulating factor, an immunomodulator, an immunomodulatory imide (IMiD), an anti-CTLA4 agent, an anti-LAG1 agent, an anti-OX40 agent, a GITR agonist, a CAR-T cell, a BiTE, a signal transduction inhibitor, a growth factor inhibitor, a tyrosine kinase inhibitor, an EGFR inhibitor, a histone deacetylase (HDAC) inhibitor, a proteasome inhibitor, a cell-cycle inhibitor, an anti-angiogenesis agent, a matrix-metalloproteinase (MMP) inhibitor, a hepatocyte growth factor inhibitor, a TOR inhibitor, a KDR inhibitor, a VEGF inhibitor, a HIF-1α inhibitor, a HIF-2α inhibitor, a fibroblast growth factor (FGF) inhibitor, a RAF inhibitor, a MEK inhibitor, an ERK inhibitor, a PI3K inhibitor, an AKT inhibitor, an MCL-1 inhibitor, a BCL-2 inhibitor, an SHP2 inhibitor, a HER-2 inhibitor, a BRAF-inhibitor, a gene expression modulator, an autophagy inhibitor, an apoptosis inducer, an antiproliferative agent, and a glycolysis inhibitor.

In one embodiment, the additional anti-cancer agent(s) is a chemotherapeutic agent. Non-limiting examples of chemotherapeutic agents include mitotic inhibitors and plant alkaloids, alkylating agents, anti-metabolites, platinum analogs, enzymes, topoisomerase inhibitors, retinoids, aziridines, and antibiotics.

Non-limiting examples of mitotic inhibitors and plant alkaloids include taxanes such as cabazitaxel, docetaxel, larotaxel, ortataxel, paclitaxel, and tesetaxel; demecolcine; epothilone; eribulin; etoposide (VP-16); etoposide phosphate; navelbine; noscapine; teniposide; thaliblastine; vinblastine; vincristine; vindesine; vinflunine; and vinorelbine.

Non-limiting examples of alkylating agents include nitrogen mustards such as chlorambucil, chlornaphazine, cholophosphamide, cytophosphane, estramustine, ifosfamide, mannomustine, mechlorethamine, mechlorethamine oxide hydrochloride, melphalan, novembichin, phenesterine, prednimustine, tris(2-chloroethyl)amine, trofosfamide, and uracil mustard; alkyl sulfonates such as busulfan, improsulfan, and piposulfan; nitrosoureas such as carmustine, chlorozotocin, fotemustine, lomustine, nimustine, ranimustine, streptozotocin, and TA-07; ethylenimines and methylamelamines such as altretamine, thiotepa, triethylenemelamine, triethylenethiophosphaoramide, trietylenephosphoramide, and trimethylolomelamine; ambamustine; bendamustine; dacarbazine; etoglucid; irofulven; mafosfamide; mitobronitol; mitolactol; pipobroman; procarbazine; temozolomide; treosulfan; and triaziquone.

Non-limiting examples of anti-metabolites include folic acid analogues such as aminopterin, denopterin, edatrexate, methotrexate, pteropterin, raltitrexed, and trimetrexate; purine analogs such as 6-mercaptopurine, 6-thioguanine, fludarabine, forodesine, thiamiprine, and thioguanine; pyrimidine analogs such as 5-fluorouracil (5-FU), 6-azauridine, ancitabine, azacytidine, capecitabine, carmofur, cytarabine, decitabine, dideoxyuridine, doxifiuridine, doxifluridine, enocitabine, floxuridine, galocitabine, gemcitabine, and sapacitabine; 3-aminopyridine-2-carboxaldehyde thiosemicarbazone; broxuridine; cladribine; cyclophosphamide; cytarabine; emitefur; hydroxyurea; mercaptopurine; nelarabine; pemetrexed; pentostatin; tegafur; and troxacitabine.

Non-limiting examples of platinum analogs include carboplatin, cisplatin, dicycloplatin, heptaplatin, lobaplatin, nedaplatin, oxaliplatin, satraplatin, and triplatin tetranitrate.

Non-limiting examples of enzymes include asparaginase and pegaspargase.

Non-limiting examples of topoisomerase inhibitors include acridine carboxamide, amonafide, amsacrine, belotecan, elliptinium acetate, exatecan, indolocarbazole, irinotecan, lurtotecan, mitoxantrone, razoxane, rubitecan, SN-38, sobuzoxane, and topotecan.

Non-limiting examples of retinoids include alitretinoin, bexarotene, fenretinide, isotretinoin, liarozole, RII retinamide, and tretinoin.

Non-limiting examples of aziridines include benzodopa, carboquone, meturedopa, and uredopa.

Non-limiting examples of antibiotics include intercalating antibiotics; anthracenediones; anthracycline antibiotics such as aclarubicin, amrubicin, daunomycin, daunorubicin, doxorubicin, epirubicin, idarubicin, menogaril, nogalamycin, pirarubicin, and valrubicin; 6-diazo-5-oxo-L-norleucine; aclacinomysins; actinomycin; authramycin; azaserine; bleomycins; cactinomycin; calicheamicin; carabicin; carminomycin; carzinophilin; chromomycins; dactinomycin; detorubicin; esorubicin; esperamicins; geldanamycin; marcellomycin; mitomycins; mitomycin C; mycophenolic acid; olivomycins; novantrone; peplomycin; porfiromycin; potfiromycin; puromycin; quelamycin; rebeccamycin; rodorubicin; streptonigrin; streptozocin; tanespimycin; tubercidin; ubenimex; zinostatin; zinostatin stimalamer; and zorubicin.

In one embodiment, the additional anti-cancer agent(s) is a hormonal and/or anti-hormonal agent (i.e., hormone therapy). Non-limiting examples of hormonal and anti-hormonal agents include anti-androgens such as abiraterone, apalutamide, bicalutamide, darolutamide, enzalutamide, flutamide, goserelin, leuprolide, and nilutamide; anti-estrogens such as 4-hydroxy tamoxifen, aromatase inhibiting 4(5)-imidazoles, EM-800, fosfestrol, fulvestrant, keoxifene, LY 117018, onapristone, raloxifene, tamoxifen, toremifene, and trioxifene; anti-adrenals such as aminoglutethimide, dexaminoglutethimide, mitotane, and trilostane; androgens such as calusterone, dromostanolone propionate, epitiostanol, mepitiostane, and testolactone; abarelix; anastrozole; cetrorelix; deslorelin; exemestane; fadrozole; finasteride; formestane; histrelin (RL 0903); human chorionic gonadotropin; lanreotide; LDI 200 (Milkhaus); letrozole; leuprorelin; mifepristone; nafarelin; nafoxidine; osaterone; prednisone; thyrotropin alfa; and triptorelin.

In one embodiment, the additional anti-cancer agent(s) is an immunotherapeutic agent (i.e., immunotherapy). Non-limiting examples of immunotherapeutic agents include biological response modifiers, cytokine inhibitors, tumor vaccines, monoclonal antibodies, immune checkpoint inhibitors, colony-stimulating factors, and immunomodulators.

Non-limiting examples of biological response modifiers, including cytokine inhibitors (cytokines) such as interferons and interleukins, include interferon alfa/interferon alpha such as interferon alfa-2, interferon alfa-2a, interferon alfa-2b, interferon alfa-n1, interferon alfa-n3, interferon alfacon-1, peginterferon alfa-2a, peginterferon alfa-2b, and leukocyte alpha interferon; interferon beta such as interferon beta-1a, and interferon beta-1b; interferon gamma such as natural interferon gamma-1a, and interferon gamma-1b; aldesleukin; interleukin-1 beta; interleukin-2; oprelvekin; sonermin; tasonermin; and virulizin.

Non-limiting examples of tumor vaccines include APC 8015, AVICINE, bladder cancer vaccine, cancer vaccine (Biomira), gastrin 17 immunogen, Maruyama vaccine, melanoma lysate vaccine, melanoma oncolysate vaccine (New York Medical College), melanoma vaccine (New York University), melanoma vaccine (Sloan Kettering Institute), TICE® BCG (Bacillus Calmette-Guerin), and viral melanoma cell lysates vaccine (Royal Newcastle Hospital).

Non-limiting examples of monoclonal antibodies include abagovomab, adecatumumab, aflibercept, alemtuzumab, blinatumomab, brentuximab vedotin, CA 125 MAb (Biomira), cancer MAb (Japan Pharmaceutical Development), daclizumab, daratumumab, denosumab, edrecolomab, gemtuzumab zogamicin, HER-2 and Fc MAb (Medarex), ibritumomab tiuxetan, idiotypic 105AD7 MAb (CRC Technology), idiotypic CEA MAb (Trilex), ipilimumab, lintuzumab, LYM-1-iodine 131 MAb (Techni clone), mitumomab, moxetumomab, ofatumumab, polymorphic epithelial mucin-yttrium 90 MAb (Antisoma), ranibizumab, rituximab, and trastuzumab.

Non-limiting examples of immune checkpoint inhibitors include anti-PD-1 agents or antibodies such as cemiplimab, nivolumab, and pembrolizumab; anti-PD-L1 agents or antibodies such as atezolizumab, avelumab, and durvalumab; anti-CTLA-4 agents or antibodies such as ipilumumab; anti-LAG1 agents; and anti-OX40 agents.

Non-limiting examples of colony-stimulating factors include darbepoetin alfa, epoetin alfa, epoetin beta, filgrastim, granulocyte macrophage colony stimulating factor, lenograstim, leridistim, mirimostim, molgramostim, nartograstim, pegfilgrastim, and sargramostim.

Non-limiting examples of additional immunotherapeutic agents include BiTEs, CAR-T cells, GITR agonists, imiquimod, immunomodulatory imides (IMiDs), mismatched double stranded RNA (Ampligen), resiquimod, SRL 172, and thymalfasin.

In one embodiment, the additional anti-cancer agent(s) is a targeted therapy agent (i.e., targeted therapy). Targeted therapy agents include, for example, monoclonal antibodies and small molecule drugs. Non-limiting examples of targeted therapy agents include signal transduction inhibitors, growth factor inhibitors, tyrosine kinase inhibitors, EGFR inhibitors, histone deacetylase (HDAC) inhibitors, proteasome inhibitors, cell-cycle inhibitors, angiogenesis inhibitors, matrix-metalloproteinase (MMP) inhibitors, hepatocyte growth factor inhibitors, TOR inhibitors, KDR inhibitors, VEGF inhibitors, fibroblast growth factors (FGF) inhibitors, MEK inhibitors, ERK inhibitors, PI3K inhibitors, AKT inhibitors, MCL-1 inhibitors, BCL-2 inhibitors, SHP2 inhibitors, HER-2 inhibitors, BRAF-inhibitors, gene expression modulators, autophagy inhibitors, apoptosis inducers, antiproliferative agents, and glycolysis inhibitors.

Non-limiting examples of signal transduction inhibitors include tyrosine kinase inhibitors, multiple-kinase inhibitors, anlotinib, avapritinib, axitinib, dasatinib, dovitinib, imatinib, lenvatinib, lonidamine, nilotinib, nintedanib, pazopanib, pegvisomant, ponatinib, vandetanib, and EGFR inhibitory agents.

Non-limiting examples of EGFR inhibitory agents include small molecule antagonists of EGFR such as afatinib, brigatinib, erlotinib, gefitinib, lapatinib, and osimertinib; and antibody-based EGFR inhibitors, including any anti-EGFR antibody or antibody fragment that can partially or completely block EGFR activation by its natural ligand. Antibody-based EGFR inhibitory agents may include, for example, those described in Modjtahedi, H., et al., 1993, Br. J. Cancer 67:247-253; Teramoto, T., et al., 1996, Cancer 77:639-645; Goldstein et al, 1995, Clin. Cancer Res. 1:1311-1318; Huang, S. M., et al., 1999, Cancer Res. 15:59(8): 1935-40; and Yang, X., et al., 1999, Cancer Res. 59: 1236-1243; monoclonal antibody Mab E7.6.3 (Yang, 1999 supra); Mab C225 (ATCC Accession No. HB-8508), or an antibody or antibody fragment having the binding specificity thereof; specific antisense nucleotide or siRNA; afatinib, cetuximab; matuzumab; necitumumab; nimotuzumab; panitumumab; and zalutumumab.

Non-limiting examples of histone deacetylase (HDAC) inhibitors include belinostat, panobinostat, romidepsin, and vorinostat.

Non-limiting examples of proteasome inhibitors include bortezomib, carfilzomib, ixazomib, marizomib (salinosporamide a), and oprozomib.

Non-limiting examples of cell-cycle inhibitors, including CDK inhibitors, include abemaciclib, alvocidib, palbociclib, and ribociclib.

In one embodiment, the additional anti-cancer agent(s) is an anti-angiogenic agent (or angiogenesis inhibitor) including, but not limited to, matrix-metalloproteinase (MMP) inhibitors; VEGF inhibitors; EGFR inhibitors; TOR inhibitors such as everolimus and temsirolimus; PDGFR kinase inhibitory agents such as crenolanib; HIF-1a inhibitors such as PX 478; HIF-2α inhibitors such as belzutifan and the HIF-2α inhibitors described in WO 2015/035223; fibroblast growth factor (FGF) or FGFR inhibitory agents such as B-FGF and RG 13577; hepatocyte growth factor inhibitors; KDR inhibitors; anti-Ang1 and anti-Ang2 agents; anti-Tie2 kinase inhibitory agents; Tek antagonists (US 2003/0162712; U.S. Pat. No. 6,413,932); anti-TWEAK agents (U.S. Pat. No. 6,727,225); ADAM distintegrin domain to antagonize the binding of integrin to its ligands (US 2002/0042368); anti-eph receptor and/or anti-ephrin antibodies or antigen binding regions (U.S. Pat. Nos. 5,981,245; 5,728,813; 5,969,110; 6,596,852; 6,232,447; and 6,057,124); and anti-PDGF-BB antagonists as well as antibodies or antigen binding regions specifically binding to PDGF-BB ligands.

Non-limiting examples of matrix-metalloproteinase (MMP) inhibitors include MMP-2 (matrix-metalloproteinase 2) inhibitors, MMP-9 (matrix-metalloproteinase 9) inhibitors, prinomastat, RO 32-3555, and RS 13-0830. Examples of useful matrix metalloproteinase inhibitors are described, for example, in WO 96/33172, WO 96/27583, EP 1004578, WO 98/07697, WO 98/03516, WO 98/34918, WO 98/34915, WO 98/33768, WO 98/30566, EP 0606046, EP 0931788, WO 90/05719, WO 99/52910, WO 99/52889, WO 99/29667, WO 1999/007675, EP 1786785, EP 1181017, US 2009/0012085, U.S. Pat. Nos. 5,863,949, 5,861,510, and EP 0780386. Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e., MAP-1, MMP-3, MMP-4, MMP-5, MMP-6, MMP-7, MMP-8, MMP-10, MMP-11, MMP-12, and MMP-13).

Non-limiting examples of VEGF and VEGFR inhibitory agents include bevacizumab, cediranib, CEP 7055, CP 547632, KRN 633, orantinib, pazopanib, pegaptanib, pegaptanib octasodium, semaxanib, sorafenib, sunitinib, VEGF antagonist (Borean, Denmark), and VEGF-TRAP™.

The additional anti-cancer agent(s) may also be another anti-angiogenic agent including, but not limited to, 2-methoxyestradiol, AE 941, alemtuzumab, alpha-D148 Mab (Amgen, US), alphastatin, anecortave acetate, angiocidin, angiogenesis inhibitors, (SUGEN, US), angiostatin, anti-Vn Mab (Crucell, Netherlands), atiprimod, axitinib, AZD 9935, BAY RES 2690 (Bayer, Germany, BC 1 (Genoa Institute of Cancer Research, Italy), beloranib, benefin (Lane Labs, US), cabozantinib, CDP 791 (Celltech Group, UK), chondroitinase AC, cilengitide, combretastatin A4 prodrug, CP 564959 (OSI, US), CV247, CYC 381 (Harvard University, US), E 7820, EHT 0101, endostatin, enzastaurin hydrochloride, ER-68203-00 (IVAX, US), fibrinogen-E fragment, Flk-1 (ImClone Systems, US), forms of FLT 1 (VEGFR 1), FR-111142, GCS-100, GW 2286 (GlaxoSmithKline, UK), IL-8, ilomastat, IM-862, irsogladine, KM-2550 (Kyowa Hakko, Japan), lenalidomide, lenvatinib, MAb alpha5beta3 integrin, second generation (Applied Molecular Evolution, USA and Medlmmune, US), MAb VEGF (Xenova, UK), marimastat, maspin (Sosei, Japan), metastatin, motuporamine C, M-PGA, ombrabulin, OXI4503, PI 88, platelet factor 4, PPI 2458, ramucirumab, rBPI 21 and BPI-derived antiangiogenic (XOMA, US), regorafenib, SC-236, SD-7784 (Pfizer, US), SDX 103 (University of California at San Diego, US), SG 292 (Telios, US), SU-0879 (Pfizer, US), TAN-1120, TBC-1635, tesevatinib, tetrathiomolybdate, thalidomide, thrombospondin 1 inhibitor, Tie-2 ligands (Regeneron, US), tissue factor pathway inhibitors (EntreMed, US), tumor necrosis factor-alpha inhibitors, tumstatin, TZ 93, urokinase plasminogen activator inhibitors, vadimezan, vandetanib, vasostatin, vatalanib, VE-cadherin-2 antagonists, xanthorrhizol, XL 784 (Exelixis, US), ziv-aflibercept, and ZD 6126.

In embodiments, the additional anti-cancer agent(s) is an additional active agent that disrupts or inhibits RAS-RAF-ERK or PI3K-AKT-TOR signaling pathways or is a PD-1 and/or PD-L1 antagonist. In embodiments, the additional anti-cancer agent(s) is a RAF inhibitor, EGFR inhibitor, MEK inhibitor, ERK inhibitor, PI3K inhibitor, AKT inhibitor, TOR inhibitor, MCL-1 inhibitor, BCL-2 inhibitor, SHP2 inhibitor, proteasome inhibitor, or immune therapy, including monoclonal antibodies, immunomodulatory imides (IMiDs), anti-PD-1, anti-PDL-1, anti-CTLA4, anti-LAG1, and anti-OX40 agents, GITR agonists, CAR-T cells, and BiTEs.

Non-limiting examples of RAF inhibitors include dabrafenib, encorafenib, regorafenib, sorafenib, and vemurafenib.

Non-limiting examples of MEK inhibitors include binimetinib, CI-1040, cobimetinib, PD318088, PD325901, PD334581, PD98059, refametinib, selumetinib, and trametinib.

Non-limiting examples of ERK inhibitors include LY3214996, LTT462, MK-8353, SCH772984, ravoxertinib, ulixertinib, and an ERKi as described in WO 2017/068412.

Non-limiting examples of PI3K inhibitors include 17-hydroxywortmannin analogs (e.g., WO 06/044453); AEZS-136; alpelisib; AS-252424; buparlisib; CAL263; copanlisib; CUDC-907; dactolisib (WO 06/122806); demethoxyviridin; duvelisib; GNE-477; GSK1059615; IC87114; idelalisib; INK1117; LY294002; Palomid 529; paxalisib; perifosine; PI-103; PI-103 hydrochloride; pictilisib (e.g., WO 09/036,082; WO 09/055,730); PIK 90; PWT33597; SF1126; sonolisib; TGI 00-115; TGX-221; XL147; XL-765; wortmannin; and ZSTK474.

Non-limiting examples of AKT inhibitors include Akt-1-1 (inhibits Aktl) (Barnett et al. (2005) Biochem. J., 385 (Pt. 2), 399-408); Akt-1-1,2 (Barnett et al. (2005) Biochem. J. 385 (Pt. 2), 399-408); API-59CJ-Ome (e.g., Jin et al. (2004) Br. J. Cancer 91, 1808-12); 1-H-imidazo[4,5-c]pyridinyl compounds (e.g., WO05011700); indole-3-carbinol and derivatives thereof (e.g., U.S. Pat. No. 6,656,963; Sarkar and Li (2004) J Nutr. 134(12 Suppl), 3493S-3498S); perifosine, Dasmahapatra et al. (2004) Clin. Cancer Res. 10(15), 5242-52, 2004); phosphatidylinositol ether lipid analogues (e.g., Gills and Dennis (2004) Expert. Opin. Investig. Drugs 13, 787-97); triciribine (Yang et al. (2004) Cancer Res. 64, 4394-9); imidazooxazone compounds including trans-3-amino-1-methyl-3-[4-(3-phenyl-5H-imidazo[1,2-c]pyrido[3,4-e][1,3]oxazin-2-yl)phenyl]-cyclobutanol hydrochloride (WO 2012/137870); afuresertib; capivasertib; MK2206; patasertib, and those disclosed in WO 2011/082270 and WO 2012/177844.

Non-limiting examples of TOR inhibitors include deforolimus; ATP-competitive TORC1/TORC2 inhibitors, including PI-103, PP242, PP30, and Torin 1; TOR inhibitors in FKBP12 enhancer, rapamycins and derivatives thereof, including temsirolimus, everolimus, WO 9409010; rapalogs, e.g. as disclosed in WO 98/02441 and WO 01/14387, e.g. AP23573, AP23464, or AP23841; 40-(2-hydroxyethyl)rapamycin, 40-[3-hydroxy(hydroxymethyl)methylpropanoate]-rapamycin; 40-epi-(tetrazolyl)-rapamycin (also called ABT578); 32-deoxorapamycin; 16-pentynyloxy-32(S)-dihydrorapanycin, and other derivatives disclosed in WO 05/005434; derivatives disclosed in U.S. Pat. No. 5,258,389, WO 94/090101, WO 92/05179, U.S. Pat. Nos. 5,118,677, 5,118,678, 5,100,883, 5,151,413, 5,120,842, WO 93/111130, WO 94/02136, WO 94/02485, WO 95/14023, WO 94/02136, WO 95/16691, WO 96/41807, WO 96/41807 and U.S. Pat. No. 5,256,790; and phosphorus-containing rapamycin derivatives (e.g., WO 05/016252).

Non-limiting examples of MCL-1 inhibitors include AMG-176, MIK665, and S63845.

Non-limiting examples of SHP2 inhibitors include SHP2 inhibitors described in WO 2019/167000 and WO 2020/022323.

Additional non-limiting examples of anti-cancer agents that are suitable for use include 2-ethylhydrazide, 2,2′,2″-trichlorotriethylamine, ABVD, aceglatone, acemannan, aldophosphamide glycoside, alpharadin, amifostine, aminolevulinic acid, anagrelide, ANCER, ancestim, anti-CD22 immunotoxins, antitumorigenic herbs, apaziquone, arglabin, arsenic trioxide, azathioprine, BAM 002 (Novelos), bcl-2 (Genta), bestrabucil, biricodar, bisantrene, bromocriptine, brostallicin, bryostatin, buthionine sulfoximine, calyculin, cell-cycle nonspecific antineoplastic agents, celmoleukin, clodronate, clotrimazole, cytarabine ocfosfate, DA 3030 (Dong-A), defofamine, denileukin diftitox, dexrazoxane, diaziquone, dichloroacetic acid, dilazep, discodermolide, docosanol, doxercalciferol, edelfosine, eflornithine, EL532 (Elan), elfomithine, elsamitrucin, eniluracil, etanidazole, exisulind, ferruginol, folic acid replenisher such as frolinic acid, gacytosine, gallium nitrate, gimeracil/oteracil/tegafur combination (S-1), glycopine, histamine dihydrochloride, HIT diclofenac, HLA-B7 gene therapy (Vical), human fetal alpha fetoprotein, ibandronate, ibandronic acid, ICE chemotherapy regimen, imexon, iobenguane, IT-101 (CRLX101), laniquidar, LC 9018 (Yakult), leflunomide, lentinan, levamisole+fluorouracil, lovastatin, lucanthone, masoprocol, melarsoprol, metoclopramide, miltefosine, miproxifene, mitoguazone, mitozolomide, mopidamol, motexafin gadolinium, MX6 (Galderma), naloxone+pentazocine, nitracrine, nolatrexed, NSC 631570 octreotide (Ukrain), olaparib, P-30 protein, PAC-1, palifermin, pamidronate, pamidronic acid, pentosan polysulfate sodium, phenamet, picibanil, pixantrone, platinum, podophyllinic acid, porfimer sodium, PSK (Polysaccharide-K), rabbit antithymocyte polyclonal antibody, rasburiembodiment, retinoic acid, rhenium Re 186 etidronate, romurtide, samarium (153 Sm) lexidronam, sizofiran, sodium phenylacetate, sparfosic acid, spirogermanium, strontium-89 chloride, suramin, swainsonine, talaporfin, tariquidar, tazarotene, tegafur-uracil, temoporfin, tenuazonic acid, tetrachlorodecaoxide, thrombopoietin, tin ethyl etiopurpurin, tirapazamine, TLC ELL-12, tositumomab-iodine 131, trifluridine and tipiracil combination, troponin I (Harvard University, US), urethan, valspodar, verteporfin, zoledronic acid, and zosuquidar.

The present disclosure further provides a method for using the compounds of Formula (I) or pharmaceutical compositions provided herein, in combination with radiation therapy to treat cancer. Techniques for administering radiation therapy are known in the art, and these techniques can be used in the combination therapy described herein. The administration of the compound of Formula (I) in this combination therapy can be determined as described herein.

Radiation therapy can be administered through one of several methods, or a combination of methods, including, without limitation, external-beam therapy, internal radiation therapy, implant radiation, stereotactic radiosurgery, systemic radiation therapy, radiotherapy and permanent or temporary interstitial brachy therapy. The term “brachytherapy,” as used herein, refers to radiation therapy delivered by a spatially confined radioactive material inserted into the body at or near a tumor or other proliferative tissue disease site. The term is intended, without limitation, to include exposure to radioactive isotopes (e.g., At-211, I-131, I-125, Y-90, Re-186, Re-188, Sm-153, Bi-212, P-32, and radioactive isotopes of Lu). Suitable radiation sources for use as a cell conditioner of the present disclosure include both solids and liquids. By way of non-limiting example, the radiation source can be a radionuclide, such as 1-125, I-131, Yb-169, Ir-192 as a solid source, 1-125 as a solid source, or other radionuclides that emit photons, beta particles, gamma radiation, or other therapeutic rays. The radioactive material can also be a fluid made from any solution of radionuclide(s), e.g., a solution of 1-125 or 1-131, or a radioactive fluid can be produced using a slurry of a suitable fluid containing small particles of solid radionuclides, such as Au-198, Y-90. Moreover, the radionuclide(s) can be embodied in a gel or radioactive microspheres.

The present disclosure also provides methods for combination therapies in which the additional active agent is known to modulate other pathways, or other components of the same pathway, or even overlapping sets of target enzymes which are used in combination with a compound of Formula (I), or a pharmaceutically acceptable salt thereof. In one embodiment, such therapy includes, but is not limited to, the combination of one or more compounds of Formula (I) with chemotherapeutic agents, immunotherapeutic agents, hormonal therapy agents, therapeutic antibodies, targeted therapy agents, and radiation treatment, to provide a synergistic or additive therapeutic effect.

The compounds of the disclosure can be used in combination with the agents disclosed herein or other suitable agents, depending on the condition being treated. Hence, in some embodiments the one or more compounds of the disclosure will be co-administered with other agents as described above. When used in combination therapy, the compounds described herein are administered with the second agent simultaneously or separately. This administration in combination can include simultaneous administration of the two agents in the same dosage form, simultaneous administration in separate dosage forms, and separate administration. That is, a compound of Formula (I) and any of the agents described above can be formulated together in the same dosage form and administered simultaneously. Alternatively, a compound of Formula (I) and any of the agents described above can be simultaneously administered, wherein both the agents are present in separate formulations. In another alternative, a compound of Formula (I) can be administered just followed by and any of the agents described above, or vice versa. In some embodiments of the separate administration protocol, a compound of Formula (I) and any of the agents described above are administered a few minutes apart, or a few hours apart, or a few days apart.

As one aspect of the present disclosure contemplates the treatment of the disease/conditions with a combination of pharmaceutically active compounds that may be administered separately, the disclosure further relates to combining separate pharmaceutical compositions in kit form. The kit comprises two separate pharmaceutical compositions: a compound of Formula (I), and a second pharmaceutical compound. The kit comprises a container for containing the separate compositions such as a divided bottle or a divided foil packet. Additional examples of containers include syringes, boxes, and bags. In some embodiments, the kit comprises directions for the use of the separate components. The kit form is particularly advantageous when the separate components are preferably administered in different dosage forms (e.g., oral and parenteral), are administered at different dosage intervals, or when titration of the individual components of the combination is desired by the prescribing health care professional.

The present disclosure also provides for the compound of Formula (I), or the pharmaceutically acceptable salt thereof, for use in therapy, or use of the compound of Formula (I), or the pharmaceutically acceptable salt thereof, in therapy. The present disclosure also provides for the compound of Formula (I), or the pharmaceutically acceptable salt thereof, for use in treating cancer, or use of a compound of Formula (I), or the pharmaceutically acceptable salt thereof, for treating cancer. The present disclosure also provides for the compound of Formula (I), or the pharmaceutically acceptable salt thereof, for the preparation of a medicament for the treatment of cancer, or use of the compound of Formula (I), or the pharmaceutically acceptable salt thereof, for the preparation of a medicament for the treatment of cancer. The present disclosure also provides for the compound of Formula (I), or the pharmaceutically acceptable salt thereof, and an additional anti-cancer agent, for use in the treatment of cancer, or use of the compound of Formula (I), or the pharmaceutically acceptable salt thereof, and the additional anti-cancer agent for treating cancer. The disclosure also provides the compound of Formula (I), or the pharmaceutically acceptable salt thereof, and an additional anti-cancer agent, for the preparation of a medicament for the treatment of cancer, or use of the compound of Formula (I), or the pharmaceutically acceptable salt thereof, and the additional anti-cancer agent, for the preparation of a medicament for the treatment of cancer. The present disclosure also provides for a pharmaceutical composition comprising the compound of Formula (I), or the pharmaceutically acceptable salt thereof, for use in the treatment of cancer, or use of the pharmaceutical composition comprising the compound of Formula (I), or the pharmaceutically acceptable salt thereof, for treating cancer. The present disclosure also provides for a pharmaceutical composition comprising the compound of Formula (I), or the pharmaceutically acceptable salt thereof, and an additional anti-cancer agent, for use in the treatment of cancer, or use of the pharmaceutical composition comprising the compound of Formula (I), or the pharmaceutically acceptable salt thereof, and the additional anti-cancer agent, for treating cancer.

### Methods of Preparing the Compounds of the Disclosure

The compounds described herein can be prepared according to the procedures of the following schemes and examples, using appropriate materials and are further exemplified by the following specific examples. The examples further illustrate details for the preparation of the compounds of the present disclosure. Those skilled in the art will readily understand that known variations of the conditions and processes of the following preparative procedures can be used to prepare these compounds. For instance, in some cases, the order of carrying out the steps of reaction schemes may be varied to facilitate the reaction or to avoid unwanted reaction products. These examples are provided for the purpose of further illustration only and are not intended to be limitations on the disclosure.

Throughout the synthetic schemes and examples, abbreviations and acronyms may be used with the following meanings unless otherwise indicated: s: singlet, d: doublet, t: triplet, q: quartet, sep: septet, dd: double doublet, dt: double triplet, td: triple doublet, tt: triple triplet, ddd: double double doublet, ddt: double double triplet, dtd: double triple doublet, tdd: triple double doublet, m: multiplet, br: broad, brs: broad singlet, tert: tertiary, DMSO-d₆: deuterated dimethyl sulfoxide, CDCl₃: deuterated chloroform, CD₃OD: deuterated methanol, THF: tetrahydrofuran, DMF: N,N-dimethylformamide, DMSO: dimethyl sulfoxide, DCM: dichloromethane, IPE: diisopropyl ether, MTBE: methyl tert-butyl ether, EtOAc: ethyl acetate, AcOH: acetic acid, TFA: trifluoroacetic acid, MeOH: methanol, EtOH: ethanol, DIAD: diisopropyl azodicarboxylate, TMAD: N,N,N′,N′-tetramethylazodicarboxamide, Et₃N: triethylamine, DIEA: N,N-diisopropylethylamine, RT: room temperature, NIS: N-iodosuccinimide, Pd(dppf)Cl₂: [1,1′-Bis(diphenylphosphino)ferrocene]dichloropalladium(II), and HATU: 1-[Bis(dimethylamino)methylene]-1H-1,2,3-triazolo[4,5-b]pyridinium 3-Oxide Hexafluorophosphate, KOAc: potassium acetate.

The reagents used in the Examples are commercially available products unless indicated otherwise. Prepacked columns manufactured by Shoko Scientific Co., Ltd., or Biotage were used in silica gel column chromatography and basic silica gel column chromatography. AVANCE NEO 400 spectrometer (400 MHz; BRUKER) and AVANCE III HD 500 spectrometer (500 MHz; BRUKER) were used for NMR spectra. For a deuterated solvent containing tetramethylsilane, tetramethylsilane was used as the internal reference. For other cases, measurement was performed using an NMR solvent as the internal reference. All δ values are indicated in ppm. Microwave reaction was performed using an Initiator (trademark) manufactured by Biotage. XSelect CSH C18 OBD Prep Columns manufactured by Waters were used for preparative reversed-phase HPLC.

SFC Columns used in the chiral resolution of stereoisomers are summarized in the following Table:

| | SFC Column |
|---|---|
| SFC Column | Abbreviation |
| Daicel Chiralcel OD-H (30 mm × 250 mm, 5 um) | Column A |
| Daicel Chiralcel OJ (30 mm × 250 mm, 10 um) | Column B |
| Daicel Chiralpak AS (50 mm × 250 mm, 10 um) | Column C |

### Intermediate A6-2: (S)-((1-((benzyloxy)methyl)-2,2-difluorocyclopropyl)methoxy)(tert-butyl)diphenylsilane

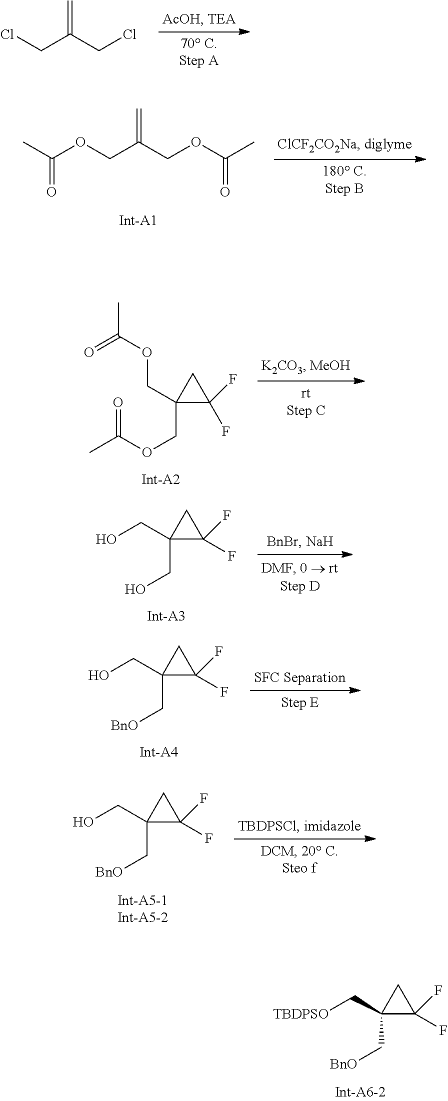

### Step A: 2-[(acetyloxy)methyl]prop-2-en-1-yl acetate (Int-A1)

A 5 L 4-necked round-bottom flask was charged with 3-chloro-2-(chloromethyl)prop-1-ene (600 g, 4.80 mol), triethylamine (1.46 kg, 14.40 mol), and acetic acid (721 g, 12.0 mol). The resulting solution was stirred overnight at 70° C. The reaction mixture was cooled to room temperature and quenched by the addition of 3 L of water. The resulting solution was extracted with ethyl acetate (3×1 L) and the combined organic layers were washed with brine solution (2×1 L). The organic layers were dried over anhydrous sodium sulfate. The dried solution was filtered and the filtrate was concentrated. The residue was purified by silica gel column with ethyl acetate/petroleum ether (1:6) to provide 2-[(acetyloxy)methyl]prop-2-en-1-yl acetate (Int-A1).

### Step B: [1-[(acetyloxy)methyl]-2,2-difluorocyclopropyl]methyl acetate (Int-A2)

Into a 20-L 4-necked round-bottom flask and maintained with an inert atmosphere of nitrogen was placed a solution of 2-[(acetyloxy)methyl]prop-2-en-1-yl acetate (Int-A1) (600 g, 3.48 mol) in diglyme (5 L). This was followed by the addition of a solution of ClCF₂CO₂Na (2.65 kg, 17.4 mol) in diglyme (5 L) dropwise with stirring at 180° C. over 5 h. The resulting solution was stirred for 1 h at 180° C. The reaction mixture was cooled to room temperature and quenched by the addition of H₂O (5 L). The resulting solution was extracted with petroleum ether (4×2 L) and the organic layers were combined. The combined organic layers were washed with water (3×2 L) and dried over anhydrous sodium sulfate. The dried solution was filtered and the filtrate was concentrated to dryness to afford [1-[(acetyloxy)methyl]-2,2-difluorocyclopropyl]methyl acetate (Int-A2), which was used directly into the next step without purification.

### Step C: 2,2-difluoro-1-(hydroxymethyl)cyclopropyl]methanol (Int-A3)

Into a 20-L 4-necked round-bottomed flask were placed [1-[(acetyloxy)methyl]-2,2-difluorocyclopropyl]methyl acetate (Int-A2) (800 g, 3.60 mol), MeOH (10 L) and K₂CO₃ (995 g, 7.20 mol). The resulting solution was stirred overnight at room temperature. The solids were filtered out. The filtrate was concentrated. The resulting mixture was then diluted by the addition of water (2 L). The resulting solution was extracted with ethyl acetate (5×1 L). The organic layers were combined and dried over anhydrous sodium sulfate. The dried solution was filtered and the filtrate was concentrated under vacuum. The residue was purified by silica gel column chromatography with ethyl acetate/petroleum ether (1:1) to afford [2,2-difluoro-1-(hydroxymethyl)cyclopropyl]methanol (Int-A3). ¹H NMR (300 MHz, DMSO-d₆) δ 1.30 (t, J=8.8 Hz, 2H), 3.52 (m, 4H), 4.79 (t, J=5.6 Hz, 2H).

### Step D: (1-((benzyloxy)methyl)-2,2-difluorocyclopropyl)methanol (Int-A4)

A 500 mL single neck round bottom flask fitted with a pour-through nitrogen adapter was purged with nitrogen and then charged with sodium hydride (4.52 g, 113 mmol) and N,N-dimethylformamide (100 mL). The suspension was cooled to 0° C. Solid [2,2-difluoro-1-(hydroxymethyl)cyclopropyl]methanol (Int-A3) (12.0 g, 87 mmol) was added portion wise. The mixture was stirred while warming to rt for 1 h. The resultant reaction mixture was cooled to 0° C. and treated with a solution of benzyl bromide (10.3 mL, 87 mmol) in N,N-dimethylformamide (10 mL). The mixture was stirred at rt for 1 h and then treated with saturated aqueous ammonium chloride (10 mL) and water (10 mL). The mixture was partitioned between ethyl acetate (75 mL) and water (75 mL). The organic layer was washed with 1 wt % aqueous LiCl (30 mL×3), dried with anhydrous sodium sulfate, filtered and the filtrate was concentrated. Purification by column chromatography on silica gel (220 g, 0 to 40% EtOAc/hexanes) afforded (1-((benzyloxy)methyl)-2,2-difluorocyclopropyl)methanol (Int-A4). ¹H NMR (499 MHz, CD₃OD) δ 7.39-7.32 (m, 4H), 7.32-7.26 (m, 1H), 4.62-4.49 (m, 2H), 3.79-3.64 (m, 3H), 3.60 (dd, J=10.4, 2.1 Hz, 1H), 1.35 (dddd, J=29.1, 12.5, 8.0, 4.5 Hz, 2H).

### Step E: (1-((benzyloxy)methyl)-2,2-difluorocyclopropyl)methanol (Int-A5)

Racemic (1-((benzyloxy)methyl)-2,2-difluorocyclopropyl)methanol (Int-A4) was resolved using SFC chiral chromatography (Column B; Conditions: 5% MeOH w/0.1% NH₄OH and 5% H₂O) to yield (R)-(1-((benzyloxy)methyl)-2,2-difluorocyclopropyl)methanol (Int-A5-1, Peak 1). ¹H NMR (499 MHz, CD₃OD) δ 7.39-7.26 (m, 5H), 4.58-4.51 (m, 2H), 3.78-3.67 (m, 3H), 3.60 (dd, J=10.4, 2.0 Hz, 1H), 1.35 (dddd, J=28.4, 12.5, 8.0, 4.5 Hz, 2H). (S)-(1-((benzyloxy)methyl)-2,2-difluorocyclopropyl)methanol (Int-A5-2, Peak 2) was also isolated.

### Step F: (R)-((1-((benzyloxy)methyl)-2,2-difluorocyclopropyl)methoxy)(tert-butyl)diphenylsilane (Int-A6-2)

To a solution of (S)-(1-((benzyloxy)methyl)-2,2-difluorocyclopropyl)methanol (Int-A5-2) (2 g, 8.76 mmol) in DCM (20 mL) was added imidazole (1.19 g, 17.5 mmol) and tert-butylchlorodiphenylsilane (2.65 g, 9.64 mmol) at 20° C. The mixture was stirred at 20° C. for 0.5 h. The mixture was diluted with DCM (60 mL), washed with H₂O (15 mL), dried over Na₂SO₄, filtered and the solvent was evaporated under reduced pressure to give the crude product. The crude product was purified by flash silica gel chromatography (ISCO®; 20 g SepaFlash® Silica Flash Column, Eluent of 5% ethyl acetate in petroleum ether gradient @ 30 mL/min) to give (R)-((1-((benzyloxy)methyl)-2,2-difluorocyclopropyl)methoxy)(tert-butyl)diphenylsilane (Int-A6-2) as colorless oil. ¹H NMR (400 MHz, CDCl₃) δ 8.02-7.50 (m, 5H), 7.48-7.23 (m, 11H), 4.56-4.45 (m, 2H), 3.81 (s, 2H), 3.70 (s, 2H), 1.28-1.15 (m, 2H), 1.05 (s, 9H).

### Intermediate B6: ((2R,7aS)-2-fluorotetrahydro-1H-pyrrolizin-7a(5H)-yl)methanol

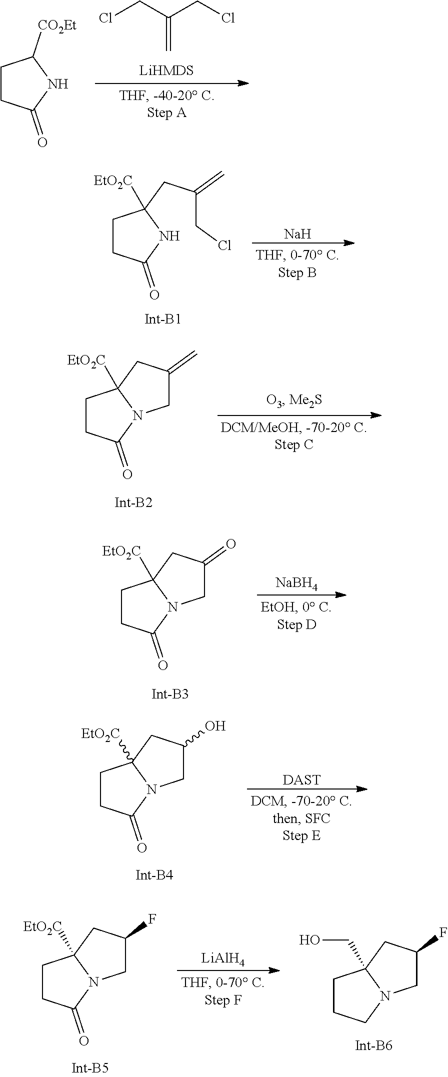

### Step A: ethyl 2-(2-(chloromethyl)allyl)-5-oxopyrrolidine-2-carboxylate (Int-B1)

LiHMDS (1.00 M, 2.55 L) was added dropwise to a solution of ethyl 5-oxopyrrolidine-2-carboxylate (200. g, 1.27 mol) and 3-chloro-2-(chloromethyl)prop-1-ene (255 g, 2.04 mol, 236 mL) in THF (2.00 L) at −40° C. under N₂. The mixture was stirred at 20° C. for 20 h.

The reaction mixture was poured into sat. NH₄Cl solution (1.00 L) and the pH of the mixture was adjusted to 6-7 with 1 N HCl. The biphasic solution was extracted with EtOAc (500 mL×3). The organic layers were combined, washed with brine (600 mL), and concentrated under reduced pressure to give a crude residue. The crude material was purified by silica gel column chromatography (Eluent: petroleum ether:ethyl acetate=50:1 to 1:1 gradient) to yield ethyl 2-(2-(chloromethyl)allyl)-5-oxopyrrolidine-2-carboxylate (Int-B1). ¹H NMR (400 MHz, CDCl₃) δ 5.06 (br d, J=17 Hz, 2H), 4.14-4.38 (m, 3H), 3.73 (br d, J=16 Hz, 1H), 3.06 (br d, J=16 Hz, 1H), 2.70-2.85 (m, 1H), 2.53-2.66 (m, 1H), 2.36-2.50 (m, 2H), 2.09-2.21 (m, 1H), 1.23-1.31 (m, 3H).

### Step B: ethyl 2-methylene-5-oxotetrahydro-1H-pyrrolizine-7a(5H)-carboxylate (Int-B2)

A solution of ethyl 2-(2-(chloromethyl)allyl)-5-oxopyrrolidine-2-carboxylate (Int-B1) (500. g, 2.03 mol) in THF (500 mL) was added dropwise to a mixture of sodium hydride (97.7 g, 2.44 mol, 60.0% purity) in THF (3.00 L) at 0° C. under nitrogen. The reaction mixture was stirred at 70° C. for 12 h under nitrogen. The reaction mixture was cooled and poured into sat. ammonium chloride solution (2.00 L) and stirred at 5° C. for 1 h. The biphasic mixture was extracted with EtOAc (600 mL×3). The combined organic layers were washed with brine (500 mL×2), dried over anhydrous Na₂SO₄, filtered, and concentrated under reduced pressure. The crude residue was purified by silica gel column chromatography (Eluent: Petroleum ether:Ethyl acetate=50:1 to 1:1) to yield ethyl 2-methylene-5-oxotetrahydro-1H-pyrrolizine-7a(5H)-carboxylate (Int-B2). ¹H NMR (400 MHz, CDCl₃) δ 4.96-5.13 (m, 2H), 4.27 (br d, J=16 Hz, 1H), 4.19 (q, J=7 Hz, 2H), 3.71 (br d, J=16 Hz, 1H), 3.04 (d, J=16 Hz, 1H), 2.69-2.83 (m, 1H), 2.59 (ddd, J=2, 9, 13 Hz, 1H), 2.40-2.52 (m, 2H), 1.96-2.22 (m, 1H), 1.26 (t, J=7 Hz, 3H).

### Step C: ethyl 2,5-dioxotetrahydro-1H-pyrrolizine-7a(5H)-carboxylate (Int-B3)

Ozone (239 mmol) (0.5˜1 m³/h) was bubbled into a solution of ethyl 2-methylene-5-oxotetrahydro-1H-pyrrolizine-7a(5H)-carboxylate (Int-B2) (160. g, 765 mmol in DCM (1.60 L) and MeOH (160 mL) at −70° C. for 9 h. Nitrogen was bubbled through the reaction mixture to purge excess ozone. Then, dimethyl sulfide (76.0 g, 1.22 mol) was added to the mixture at −70° C. The reaction mixture was stirred at 20° C. for 14 h. The reaction mixture was concentrated under reduced pressure to give a residue. The crude residue was purified by silica gel column chromatography (Eluent: Petroleum ether:Ethyl acetate=50:1 to 1:1) to yield ethyl 2,5-dioxotetrahydro-1H-pyrrolizine-7a(5H)-carboxylate (Int-B3). ¹H NMR (400 MHz, CDCl₃) δ 4.22 (q, J=7 Hz, 2H), 4.07-4.12 (m, 1H), 3.54 (dd, J=1, 18 Hz, 1H), 2.92-3.03 (m, 2H), 2.74-2.88 (m, 1H), 2.42-2.51 (m, 2H), 2.12-2.23 (m, 1H), 1.27 (t, J=7 Hz, 3H)

### Step D: ethyl 2-hydroxy-5-oxotetrahydro-1H-pyrrolizine-7a(5H)-carboxylate (Int-B4)

To a solution of ethyl 2,5-dioxotetrahydro-1H-pyrrolizine-7a(5H)-carboxylate (Int-B3) (200 g, 947 mmol) in EtOH (2.00 L) at 0° C. under N₂ was added NaBH₄ (10.8 g, 284 mmol). The reaction mixture was stirred at 0° C. for 10 min. The reaction mixture was quenched by addition of sat. NH₄Cl (50.0 mL) at 5° C. and the mixture was stirred at 5° C. for 0.5 h. The reaction mixture was concentrated under reduced pressure. The crude product from four identical reactions was then combined. The crude residue was purified by silica gel column chromatography (Eluent: Petroleum ether:Ethyl acetate=50:1 to 1:1) to yield ethyl 2-hydroxy-5-oxotetrahydro-1H-pyrrolizine-7a(5H)-carboxylate (Int-B4). ¹H NMR (400 MHz, CDCl₃) δ 4.54-4.70 (m, 1H), 4.16-4.31 (m, 2H), 3.93 (dd, J=6.0, 13 Hz, 1H), 3.09 (d, J=13 Hz, 1H), 2.75-2.90 (m, 1H), 2.39-2.63 (m, 4H), 2.01-2.13 (m, 1H), 1.83 (dd, J=6, 14 Hz, 1H), 1.29 (t, J=7 Hz, 3H).

### Step E: ethyl (2R,7aS)-2-fluoro-5-oxotetrahydro-1H-pyrrolizine-7a(5H)-carboxylate (Int-B5)

To a solution of ethyl 2-hydroxy-5-oxotetrahydro-1H-pyrrolizine-7a(5H)-carboxylate (Int-B4) in DCM (600 mL) was added DAST (90.7 g, 563 mmol, 74.4 mL) dropwise at −70° C. under N₂. The reaction mixture was warmed to 20° C. and stirred for 16 h. The reaction was quenched by the addition of EtOH (50.0 mL) at 10° C., and then diluted with water (300 mL) and extracted with DCM (200 mL×2). The combined organic layers were washed with brine (200 mL), dried over anhydrous Na₂SO₄, filtered, and concentrated under reduced pressure. The crude product was combined from six identical reactions and purified by silica gel column chromatography (Petroleum ether:Ethyl Acetate=50:1 to 1:1). This material was further purified by prep-HPLC with HCl modifier (MeCN/water with 0.05% HCl modifier). The racemic mixture was resolved using chiral SFC (Column C; Conditions: 0.1% NH₄OH in EtOH) to yield ethyl (2R,7aS)-2-fluoro-5-oxotetrahydro-1H-pyrrolizine-7a(5H)-carboxylate (Int-B5, Peak 2). ¹H NMR (400 MHz, CDCl₃): δ 5.16-5.43 (m, 1H), 4.14-4.27 (m, 3H), 3.06-3.26 (m, 1H), 2.57-2.85 (m, 3H), 2.38-2.50 (m, 1H), 2.07-−2.30 (m, 2H), 1.28 (t, J=7 Hz, 3H).

### Step F: ((2R,7aS)-2-fluorotetrahydro-1H-pyrrolizin-7a(5H)-yl)methanol (Int-B6)

A solution of ethyl (2R,7aS)-2-fluoro-5-oxotetrahydro-1H-pyrrolizine-7a(5H)-carboxylate (Int-B5) (82.0 g, 381 mmol) in THF (300 mL) was added to the mixture of LAH (21.7 g, 571 mmol) in THF (520 mL) at 0° C. under nitrogen. The reaction mixture was warmed to 70° C. and stirred for 3 h. The reaction mixture was cooled to 0° C. and quenched by the addition of Na₂SO₄·10 H₂O at 0° C. under nitrogen. The reaction mixture was stirred at 20° C. for 0.5 h and then filtered. The filter cake was washed with EtOAc (600 mL×5) and the filtrate was dried over anhydrous Mg₂SO₄. The mixture was filtered and the filtrate concentrated under reduced pressure to give a residue. The crude residue was purified by silica gel column chromatography (SiO₂, DCM:Methanol=100:1 to 10:1) to yield ((2R,7aS)-2-fluorotetrahydro-1H-pyrrolizin-7a(5H)-yl)methanol (Int-B6). ¹H NMR (400 MHz, CDCl₃): δ 5.06-5.34 (m, 1H), 3.25 (s, 2H), 3.08-3.23 (m, 3H), 2.85-3.08 (m, 2H), 2.00-2.12 (m, 2H), 1.74-1.93 (m, 4H).

### Example 1: 2-((S)-4-((S)-7-(8-ethynyl-3-hydroxynaphthalen-1-yl)-2-((1-(morpholinomethyl)cyclopropyl)methoxy)-7,8-dihydro-5H-pyrano[4,3-d]pyrimidin-4-yl)1-((E)-4-(4-methoxypiperidin-1-yl)but-2-enoyl)piperazin-2-yl)acetonitrile (Ex. 1)

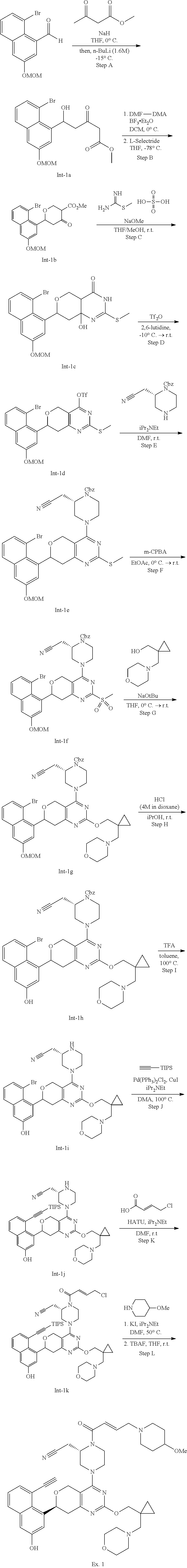

### Step A: Methyl 5-(8-bromo-3-(methoxymethoxy)naphthalen-1-yl)-5-hydroxy-3-oxopentanoate (Int-1a)

To a solution of methyl acetoacetate (1.5 mL, 14 mmol) in THF (40 mL) was added sodium hydride (0.56 g, 14 mmol, 60% dispersion in paraffin liquid) at 0° C., and the mixture was stirred for 30 min at 0° C. To the mixture was dropwise added n-butyl lithium (8.9 mL, 14 mmol, 1.6 M in hexane) at −15° C., and the mixture was stirred for an hour at the same temperature. To the mixture was added 8-bromo-3-(methoxymethoxy)-1-naphthaldehyde (2.6 g, 8.8 mmol) at −15° C., and the mixture was stirred for 30 min at the same temperature. To the mixture was added saturated aqueous NH₄Cl solution, and diluted with EtOAc. The organic layer was separated and washed with H₂O and concentrated. The residue was purified by column chromatography on silica gel (gradient elution, 20-60% EtOAc/Hexane) to give the title compound (Int-1a).

### Step B: Methyl 6-(8-bromo-3-(methoxymethoxy)naphthalen-1-yl)-4-oxotetrahydro-2H-pyran-3-carboxylate (Int-1b)

To a solution of methyl 5-(8-bromo-3-(methoxymethoxy)naphthalen-1-yl)-5-hydroxy-3-oxopentanoate (Int-1a) (3.0 g, 7.2 mmol) in dichloromethane (36 mL) was added N,N-dimethylformamide dimethyl acetal (1.1 mL, 8.0 mmol) at room temperature, and the mixture was stirred for 2 hours. To the mixture was added boron trifluoride-ethyl ether complex (0.91 mL, 7.2 mmol) at 0° C., and then the mixture was diluted with EtOAc. After removal of dichloromethane in vacuo, saturated aqueous NaHCO₃ solution was added to the mixture, and the organic layer was separated and washed with H₂O and concentrated. The residue was used for the next reaction without purification.

To a solution of the above product in THF (36 mL) was added L-Selectride solution (7.2 mL, 7.2 mmol, 1M in THF) at −78° C., and the mixture was stirred for 3 hours at the same temperature. Additional L-Selectride solution (0.5 mL) was added, and then saturated aqueous NH₄Cl solution was added. The mixture was extracted with EtOAc, and the organic layer was separated and washed with H₂O and concentrated. The residue was purified by column chromatography on silica gel (gradient elution, 0-30% EtOAc/Hexane) to give the title compound (Int-1b). ESI-MS m/z 423, 425 (M+H)⁺.

### Step C: 7-(8-bromo-3-(methoxymethoxy)naphthalen-1-yl)-8a-hydroxy-2-(methylthio)-3,4a,5,7,8,8a-hexahydro-4H-pyrano[4,3-d]pyrimidin-4-one (Int-1c)

To a solution of methyl 6-(8-bromo-3-(methoxymethoxy)naphthalen-1-yl)-4-oxotetrahydro-2H-pyran-3-carboxylate (Int-1b) (2.7 g, 5.3 mmol) and S-methylisothiourea sulfate (3.0 g, 11 mmol) in MeOH (60 mL) and THF (20 mL) was added sodium methoxide (1.4 g, 27 mmol) at room temperature, and the mixture was stirred for 2 hours at the same temperature. Solvent was removed under reduced pressure, and dichloromethane was added to the residue. The mixture was washed with water and concentrated to give the title compound (Int-1c). ESI-MS m/z 481, 483 (M+H)⁺.

### Step D: 7-(8-bromo-3-(methoxymethoxy)naphthalen-1-yl)-2-(methylthio)-7,8-dihydro-5H-pyrano[4,3-d]pyrimidin-4-yl trifluoromethanesulfonate (Int-1d)

To a solution of 7-(8-bromo-3-(methoxymethoxy)naphthalen-1-yl)-8a-hydroxy-2-(methylthio)-3,4a,5,7,8,8a-hexahydro-4H-pyrano[4,3-d]pyrimidin-4-one (Int-1c) (1.0 g, 2.1 mmol) in 2,6-lutidine (20 mL) was added trifluoromethanesulfonic anhydride (2.1 mL, 12 mmol) at −10° C., and the mixture was stirred for 10 min at room temperature. After cooling to −10° C., additional trifluoromethanesulfonic anhydride (2.1 mL, 12 mmol) was added, and the mixture was allowed to warm to room temperature. After the reaction was completed, to the mixture was added EtOAc and aqueous HCl solution (1M), followed by extraction with EtOAc. The organic layer was separated and washed with aqueous HCl solution (1M) and H₂O, and concentrated. The residue was purified by column chromatography on silica gel (gradient elution, 0-30% EtOAc/Hexane) to give the title compound (Int-1d). ESI-MS m/z 595, 597 (M+H)⁺.

### Step E: Benzyl (2S)-4-(7-(8-bromo-3-(methoxymethoxy)naphthalen-1-yl)-2-(methylthio)-7,8-dihydro-5H-pyrano[4,3-d]pyrimidin-4-yl)-2-(cyanomethyl)piperazine-1-carboxylate (Int-1e)

To a solution of 7-(8-bromo-3-(methoxymethoxy)naphthalen-1-yl)-2-(methylthio)-7,8-dihydro-5H-pyrano[4,3-d]pyrimidin-4-yl trifluoromethanesulfonate (Int-1d) (500 mg, 0.84 mmol) in DMF (5 mL) was added benzyl (S)-2-(cyanomethyl)piperazine-1-carboxylate (260 mg, 1.0 mmol) and N,N-diisopropylethylamine (0.43 mL, 2.5 mmol) at room temperature. After stirring at room temperature for an hour, the reaction mixture was diluted with EtOAc and saturated aqueous NH₄Cl solution. The organic layer was separated and washed with H₂O and concentrated. The residue was purified by column chromatography on silica gel (gradient elution, 0-100% EtOAc/Hexane) to give the title compound (Int-1e). ESI-MS m/z 704, 706 (M+H)⁺.

### Step F: Benzyl (2S)-4-(7-(8-bromo-3-(methoxymethoxy)naphthalen-1-yl)-2-(methylsulfonyl)-7,8-dihydro-5H-pyrano[4,3-d]pyrimidin-4-yl)-2-(cyanomethyl)piperazine-1-carboxylate (Int-1f)

To a solution of benzyl (2S)-4-(7-(8-bromo-3-(methoxymethoxy)naphthalen-1-yl)-2-(methylthio)-7,8-dihydro-5H-pyrano[4,3-d]pyrimidin-4-yl)-2-(cyanomethyl)piperazine-1-carboxylate (Int-1e) (700 mg, 1.0 mmol) in EtOAc (10 mL) was added m-chloroperoxybenzoic acid (580 mg, 2.2 mmol, with abt. 35% water) at 0° C., and the mixture was stirred for an hour at room temperature. To the mixture was added aqueous NaHCO₃ solution, and the organic layer was separated and washed with H₂O and concentrated. The residue was purified by column chromatography on silica gel (gradient elution, 30-100% EtOAc/Hexane) to give the title compound (Int-1f). ESI-MS m/z 736, 738 (M+H)⁺.

### Step G: Benzyl (2S)-4-(7-(8-bromo-3-(methoxymethoxy)naphthalen-1-yl)-2-((1-(morpholinomethyl)cyclopropyl)methoxy)-7,8-dihydro-5H-pyrano[4,3-d]pyrimidin-4-yl)-2-(cyanomethyl)piperazine-1-carboxylate (Int-1g)

To a solution of benzyl (2S)-4-(7-(8-bromo-3-(methoxymethoxy)naphthalen-1-yl)-2-(methylsulfonyl)-7,8-dihydro-5H-pyrano[4,3-d]pyrimidin-4-yl)-2-(cyanomethyl)piperazine-1-carboxylate (Int-1f) (120 mg, 0.16 mmol) and (1-(morpholinomethyl)cyclopropyl)methanol (55 mg, 0.33 mmol) in THF (1 mL) was added sodium tert-butoxide (31 mg, 0.33 mmol) at 0° C., and the mixture was stirred for an hour at room temperature. The reaction mixture was directly loaded on amino-functionalized silica gel and purified by column chromatography (gradient elution, 40-100% EtOAc/Hexane) to give the title compound (Int-1g). The mixture was used in the next step without further purification. ESI-MS m/z 827, 829 (M+H)⁺.

### Step H: Benzyl (2S)-4-(7-(8-bromo-3-hydroxynaphthalen-1-yl)-2-((1-(morpholinomethyl)cyclopropyl)methoxy)-7,8-dihydro-5H-pyrano[4,3-d]pyrimidin-4-yl)-2-(cyanomethyl)piperazine-1-carboxylate (Int-1h)

To a solution of benzyl (2S)-4-(7-(8-bromo-3-(methoxymethoxy)naphthalen-1-yl)-2-((1-(morpholinomethyl)cyclopropyl)methoxy)-7,8-dihydro-5H-pyrano[4,3-d]pyrimidin-4-yl)-2-(cyanomethyl)piperazine-1-carboxylate (Int-1g) (110 mg) in isopropanol (1.1 mL) was added HCl solution (1.1 mL, 4 M in 1,4-dioxane) at room temperature. After stirring for 30 min, aqueous NaHCO₃ solution was added to the reaction mixture, and the organic layer was separated and washed with H₂O and concentrated to give the title compound (Int-1h). The crude compound was used in the next step without further purification. ESI-MS m/z 783, 785 (M+H)⁺.

### Step I: 2-((2S)-4-(7-(8-bromo-3-hydroxynaphthalen-1-yl)-2-((1-(morpholinomethyl)cyclopropyl)methoxy)-7,8-dihydro-5H-pyrano[4,3-d]pyrimidin-4-yl)piperazin-2-yl)acetonitrile (Int-1i)

To a solution of benzyl (2S)-4-(7-(8-bromo-3-hydroxynaphthalen-1-yl)-2-((1-(morpholinomethyl)cyclopropyl)methoxy)-7,8-dihydro-5H-pyrano[4,3-d]pyrimidin-4-yl)-2-(cyanomethyl)piperazine-1-carboxylate (Int-1h) (93 mg) in toluene (1.0 mL) was added trifluoroacetic acid (1.0 mL), and the mixture was stirred at 100° C. for 3 h. After the reaction was completed, aqueous NaHCO₃ solution was added to the reaction mixture, and the organic layer was separated and washed with H₂O and concentrated to give the title compound (Int-1i). The crude compound was used in the next step without further purification. ESI-MS m/z 649, 651 (M+H)⁺.

### Step J: 2-((2S)-4-(7-(3-hydroxy-8-((triisopropylsilyl)ethynyl)naphthalen-1-yl)-2-((1-(morpholinomethyl)cyclopropyl)methoxy)-7,8-dihydro-5H-pyrano[4,3-d]pyrimidin-4-yl)piperazin-2-yl)acetonitrile (Int-1j)

To a solution of 2-((2S)-4-(7-(8-bromo-3-hydroxynaphthalen-1-yl)-2-((1-(morpholinomethyl)cyclopropyl)methoxy)-7,8-dihydro-5H-pyrano[4,3-d]pyrimidin-4-yl)piperazin-2-yl)acetonitrile (Int-1i) (88 mg), copper(I) iodide (5.1 mg, 0.027 mmol), bis(triphenylphosphine)palladium(II) dichloride (19 mg, 0.027 mmol) and N,N-diisopropylethylamine (92 uL, 0.68 mmol) in DMA (1 mL) was added (triisopropylsilyl)acetylene (150 uL, 0.68 mmol) at room temperature. The vessel was evacuated and backfilled with nitrogen, and the mixture was stirred at 100° C. for an hour. After the reaction was completed, the mixture was diluted with EtOAc and water, and the organic layer was separated and washed with water and concentrated. The residue was purified by column chromatography on amine-functionalized silica gel (gradient elution, 0-40% MeOH/EtOAc) to give the title compound (Int-1j). ESI-MS m/z 751 (M+H)⁺.

### Step K: 2-((2S)-1-((E)-4-chlorobut-2-enoyl)-4-(7-(3-hydroxy-8-((triisopropylsilyl)ethynyl)naphthalen-1-yl)-2-((1-(morpholinomethyl)cyclopropyl)methoxy)-7,8-dihydro-5H-pyrano[4,3-d]pyrimidin-4-yl)piperazin-2-yl)acetonitrile (Int-1k)

To a solution of 2-((2S)-4-(7-(3-hydroxy-8-((triisopropylsilyl)ethynyl)naphthalen-1-yl)-2-((1-(morpholinomethyl)cyclopropyl)methoxy)-7,8-dihydro-5H-pyrano[4,3-d]pyrimidin-4-yl)piperazin-2-yl)acetonitrile (Int-1j) (53 mg, 0.071 mmol) in DMF (1 mL) was added 1-[Bis(dimethylamino)methylene]-1H-1,2,3-triazolo[4,5-b]pyridinium 3-oxide Hexafluorophosphate (32 mg, 0.085 mmol), (E)-4-chlorobut-2-enoic acid (10 mg, 0.085 mmol) and N,N-diisopropylethylamine (37 uL, 0.21 mmol) at room temperature. After stirring for 30 min, the reaction mixture was diluted with EtOAc, and the mixture was washed with water. The organic layer was dried over sodium sulfate. The dried solution was filtered, and the filtrate was concentrated in vacuo to give the title compound (Int-1k). The crude compound was used in the next step without further purification. ESI-MS m/z 853 (M+H)⁺.

### Step L: 2-((S)-4-((S)-7-(8-ethynyl-3-hydroxynaphthalen-1-yl)-2-((1-(morpholinomethyl)cyclopropyl)methoxy)-7,8-dihydro-5H-pyrano[4,3-d]pyrimidin-4-yl)-1-((E)-4-(4-methoxypiperidin-1-yl)but-2-enoyl)piperazin-2-yl)acetonitrile (Ex. 1)

To a solution of 2-((2S)-1-((E)-4-chlorobut-2-enoyl)-4-(7-(3-hydroxy-8-((triisopropylsilyl)ethynyl)naphthalen-1-yl)-2-((1-(morpholinomethyl)cyclopropyl)methoxy)-7,8-dihydro-5H-pyrano[4,3-d]pyrimidin-4-yl)piperazin-2-yl)acetonitrile (Int-1k) (68 mg) in DMF (1 mL) was added 4-methoxypiperidine (18 mg, 0.16 mmol), potassium iodide (16 mg, 0.096 mmol) and N,N-diisopropylethylamine (28 uL, 0.16 mmol) at room temperature. The reaction mixture was heated at 50° C. for 3 h. After the reaction was completed, the reaction mixture was diluted with EtOAc, and the mixture was washed with water. The organic layer was dried over sodium sulfate. The dried solution was filtered, and the filtrate was concentrated in vacuo. To a solution of crude compound in THF (1 mL) was added tetrabutylammonium fluoride (0.16 mL, 0.16 mmol, 1M solution in THF) at room temperature. After stirring for 15 min, the reaction mixture was diluted with EtOAc, and the mixture was washed with water. The organic layer was dried over sodium sulfate. The dried solution was filtered, and the filtrate was concentrated in vacuo. The residue was purified via reverse phase HPLC (elution: acetonitrile/water, 0.1% formic acid) to give the title compound (Ex. 1). 1H-NMR (400 MHz, CDCl₃δ 7.74 (d, J=8.4 Hz, 1H), 7.66 (d, J=7.2 Hz, 1H), 7.55-7.51 (m, 1H), 7.38-7.33 (m, 1H) 7.15-7.13 (m, 1H), 7.02-6.93 (m, 1H), 6.87-6.82 (m, 1H) 6.60-6.40 (br s, 1H), 5.03-4.92 (m, 2H), 4.79 (d, J=13.6 Hz, 1H), 4.24 (s, 2H), 4.00-3.61 (m, 8H), 3.44-3.19 (m, 3H), 3.36 (s, 3H), 3.09-2.59 (m, 5H), 2.55-2.29 (m, 7H), 2.03-1.86 (m, 2H), 1.70-1.42 (m, 4H), 1.31-1.22 (m, 4H), 0.69-0.62 (m, 2H), 0.48-0.42 (m, 2H). ESI-MS m/z 776 (M+H)⁺.

The examples in the table below were prepared in a similar manner to Ex. 1 above.

| Ex. | Structure | Compound Name | [M + H]⁺ |
|---|---|---|---|
| Ex. 2 | 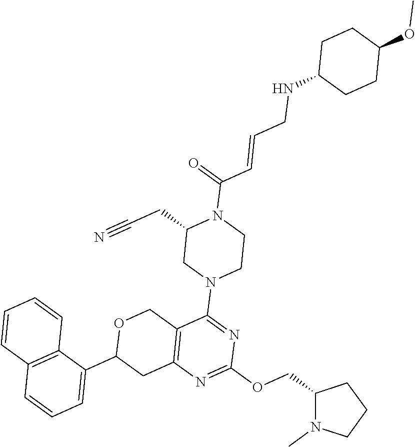 [SMILES: CO[C@H]1CC[C@H](NC/C=C/C(=O)N2CCN(c3nc(OC[C@@H]4CCCN4C)nc4c3COC(c3cccc5ccccc35)C4)C[C@@H]2CC#N)CC1] | 2-((2S)-1-((E)-4-(((1r,4S)-4-methoxycyclohexyl)amino)- but-2-enoyl)-4-(2-(((S)-1- methylpyrrolidin-2-yl)meth- oxy)-7-(naphthalen-1-yl)- 7,8-dihydro-5H-pyrano[4,3- d]pyrimidin-4-yl)piperazin- 2-yl)acetonitrile | 695 |
| Ex. 3 | 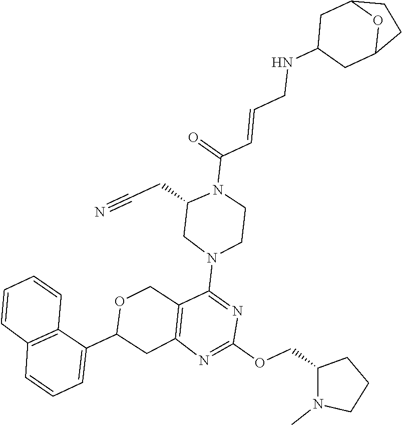 [SMILES: CN1CCC[C@H]1COc1nc2c(c(N3CCN(C(=O)/C=C/CNC4CC5CCC(C4)O5)[C@@H](CC#N)C3)n1)COC(c1cccc3ccccc13)C2] | 2-((2S)-1-((E)-4-((8-oxa- bicyclo[3.2.1]octan-3-yl)- amino)but-2-enoyl)-4-(2- (((S)-1-methylpyrrolidin-2- yl)methoxy)-7-(naphthalen- 1-yl)-7,8-dihydro-5H- pyrano[4,3-d]pyrimidin-4- yl)piperazin-2-yl)acetonitrile | 693 |
| Ex. 4 | 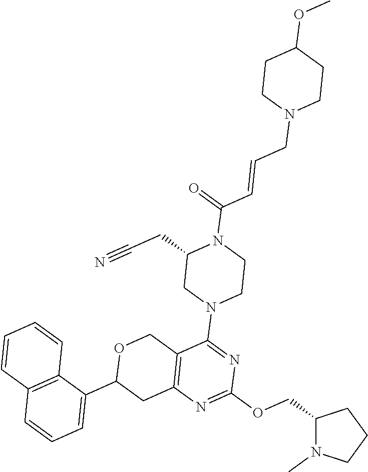 [SMILES: COC1CCN(C/C=C/C(=O)N2CCN(c3nc(OC[C@@H]4CCCN4C)nc4c3COC(c3cccc5ccccc35)C4)C[C@@H]2CC#N)CC1] | 2-((2S)-1-((E)-4-(4-methoxy- piperidin-1-yl)but-2-enoyl)- 4-(2-(((S)-1-methylpyrroli- din-2-yl)methoxy)-7-(naph- thalen-1-yl)-7,8-dihydro-5H- pyrano[4,3-d]pyrimidin-4- yl)piperazin-2-yl)acetonitrile | 681 |
| Ex. 5 | 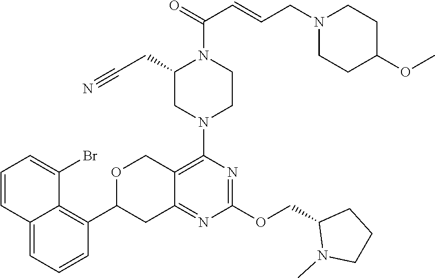 [SMILES: COC1CCN(C/C=C/C(=O)N2CCN(c3nc(OC[C@@H]4CCCN4C)nc4c3COC(c3cccc5cccc(Br)c35)C4)C[C@@H]2CC#N)CC1] | 2-((2S)-4-(7-(8-bromonaph- thalen-1-yl)-2-(((S)-1-meth- ylpyrrolidin-2-yl)methoxy)- 7,8-dihydro-5H-pyrano[4,3- d]pyrimidin-4-yl)-1-((E)-4- (4-methoxypiperidin-1-yl)- but-2-enoyl)piperazin-2-yl)- acetonitrile | 758, 760 |
| Ex. 6 | 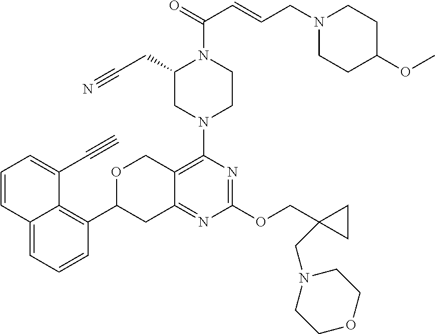 [SMILES: C#Cc1cccc2cccc(C3Cc4nc(OCC5(CN6CCOCC6)CC5)nc(N5CCN(C(=O)/C=C/CN6CCC(OC)CC6)[C@@H](CC#N)C5)c4CO3)c12] | 2-((2S)-4-(7-(8-ethynylnaph- thalen-1-yl)-2-((1-(morpho- linomethyl)cyclopropyl)- methoxy)-7,8-dihydro-5H- pyrano[4,3-d]pyrimidin-4- yl)-1-((E)-4-(4-methoxy- piperidin-1-yl)but-2-enoyl)- piperazin-2-yl)acetonitrile | 760 |
| Ex. 7 | 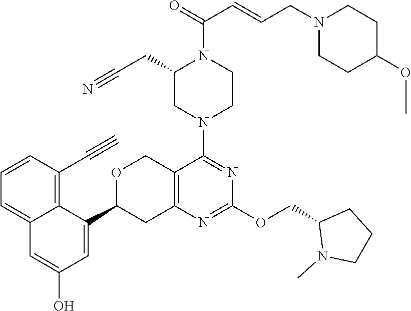 [SMILES: C#Cc1cccc2cc(O)cc([C@@H]3Cc4nc(OC[C@@H]5CCCN5C)nc(N5CCN(C(=O)/C=C/CN6CCC(OC)CC6)[C@@H](CC#N)C5)c4CO3)c12] | 2-((S)-4-((S)-7-(8-ethynyl- 3-hydroxynaphthalen-1-yl)- 2-(((S)-1-methylpyrrolidin- 2-yl)methoxy)-7,8-dihydro- 5H-pyrano[4,3-d]pyrimidin- 4-yl)-1-((E)-4-(4-methoxy- piperidin-1-yl)but-2-enoyl)- piperazin-2-yl)acetonitrile | 720 |
| Ex. 8 | 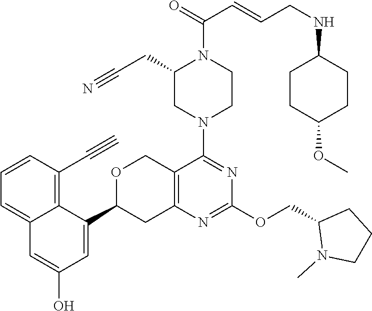 [SMILES: C#Cc1cccc2cc(O)cc([C@@H]3Cc4nc(OC[C@@H]5CCCN5C)nc(N5CCN(C(=O)/C=C/CN[C@H]6CC[C@H](OC)CC6)[C@@H](CC#N)C5)c4CO3)c12] | 2-((S)-4-((S)-7-(8-ethynyl- 3-hydroxynaphthalen-1-yl)- 2-(((S)-1-methylpyrrolidin- 2-yl)methoxy)-7,8-dihydro- 5H-pyrano[4,3-d]pyrimidin- 4-yl)-1-((E)-4-(((1r,4S)-4- methoxycyclohexyl)amino)- but-2-enoyl)piperazin-2-yl)- acetonitrile | 734 |
| Ex. 9 | 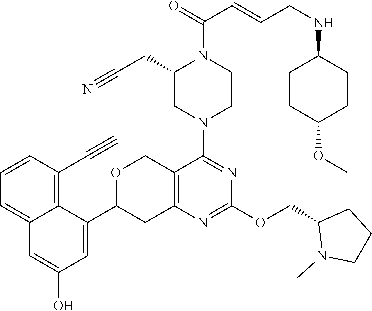 [SMILES: C#Cc1cccc2cc(O)cc(C3Cc4nc(OC[C@@H]5CCCN5C)nc(N5CCN(C(=O)/C=C/CN[C@H]6CC[C@H](OC)CC6)[C@@H](CC#N)C5)c4CO3)c12] | 2-((2S)-4-(7-(8-ethynyl-3- hydroxynaphthalen-1-yl)-2- (((S)-1-methylpyrrolidin-2- yl)methoxy)-7,8-dihydro- 5H-pyrano[4,3-d]pyrimidin- 4-yl)-1-((E)-4-(((1r,4S)-4- methoxycyclohexyl)amino)- but-2-enoyl)piperazin-2-yl)- acetonitrile | 734 |
| Ex. 10 | 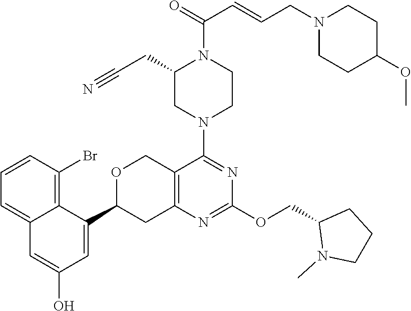 [SMILES: COC1CCN(C/C=C/C(=O)N2CCN(c3nc(OC[C@@H]4CCCN4C)nc4c3CO[C@H](c3cc(O)cc5cccc(Br)c35)C4)C[C@@H]2CC#N)CC1] | 2-((S)-4-((S)-7-(8-bromo-3- hydroxynaphthalen-1-yl)-2- (((S)-1-methylpyrrolidin-2- yl)methoxy)-7,8-dihydro- 5H-pyrano[4,3-d]pyrimidin- 4-yl)-1-((E)-4-(4-methoxy- piperidin-1-yl)but-2-enoyl)- piperazin-2-yl)acetonitrile | 774, 776 |
| Ex. 11 | 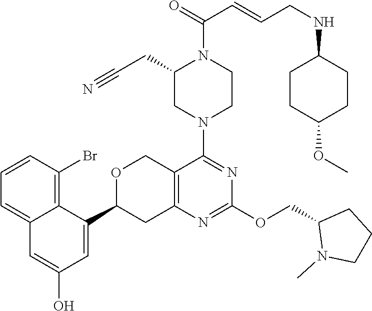 [SMILES: CO[C@H]1CC[C@H](NC/C=C/C(=O)N2CCN(c3nc(OC[C@@H]4CCCN4C)nc4c3CO[C@H](c3cc(O)cc5cccc(Br)c35)C4)C[C@@H]2CC#N)CC1] | 2-((S)-4-((S)-7-(8-bromo-3- hydroxynaphthalen-1-yl)-2- (((S)-1-methylpyrrolidin-2- yl)methoxy)-7,8-dihydro- 5H-pyrano[4,3-d]pyrimidin- 4-yl)-1-((E)-4-(((1r,4S)-4- methoxycyclohexyl)amino)- but-2-enoyl)piperazin-2-yl)- acetonitrile | 788, 790 |
| Ex. 12 | 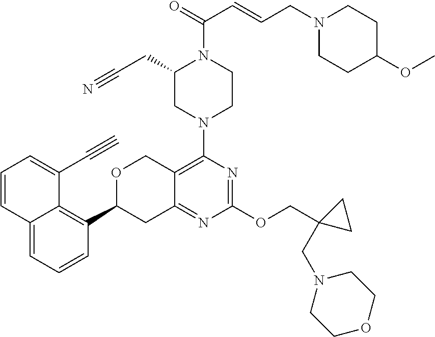 [SMILES: C#Cc1cccc2cccc([C@@H]3Cc4nc(OCC5(CN6CCOCC6)CC5)nc(N5CCN(C(=O)/C=C/CN6CCC(OC)CC6)[C@@H](CC#N)C5)c4CO3)c12] | 2-((S)-4-((S)-7-(8-ethynyl- naphthalen-1-yl)-2-((1- (morpholinomethyl)cyclo- propyl)methoxy)-7,8- dihydro-5H-pyrano[4,3- d]pyrimidin-4-yl)-1-((E)-4- (4-methoxypiperidin-1-yl)- but-2-enoyl)piperazin-2-yl)- acetonitrile | 760 |
| Ex. 13 | 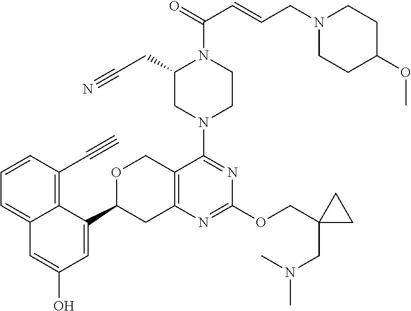 [SMILES: C#Cc1cccc2cc(O)cc([C@@H]3Cc4nc(OCC5(CN(C)C)CC5)nc(N5CCN(C(=O)/C=C/CN6CCC(OC)CC6)[C@@H](CC#N)C5)c4CO3)c12] | 2-((S)-4-((S)-2-((1-((dimeth- ylamino)methyl)cycloprop- yl)methoxy)-7-(8-ethynyl-3- hydroxynaphthalen-1-yl)- 7,8-dihydro-5H-pyrano[4,3- d]pyrimidin-4-yl)-1-((E)-4- (4-methoxypiperidin-1-yl)- but-2-enoyl)piperazin-2-yl)- acetonitrile | 734 |
| Ex. 14 | 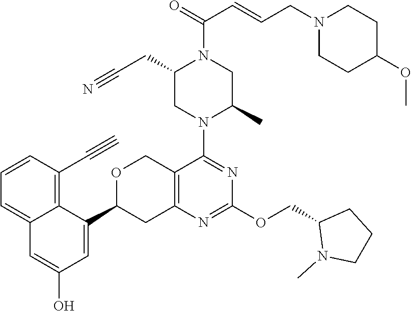 [SMILES: C#Cc1cccc2cc(O)cc([C@@H]3Cc4nc(OC[C@@H]5CCCN5C)nc(N5C[C@H](CC#N)N(C(=O)/C=C/CN6CCC(OC)CC6)C[C@H]5C)c4CO3)c12] | 2-((2S,5R)-4-((S)-7-(8- ethynyl-3-hydroxynaph- thalen-1-yl)-2-(((S)-1-meth- ylpyrrolidin-2-yl)methoxy)- 7,8-dihydro-5H-pyrano[4,3- d]pyrimidin-4-yl)-1-((E)-4- (4-methoxypiperidin-1-yl)- but-2-enoyl)-5-methylpiper- azin-2-yl)acetonitrile | 734 |
| Ex. 15 | 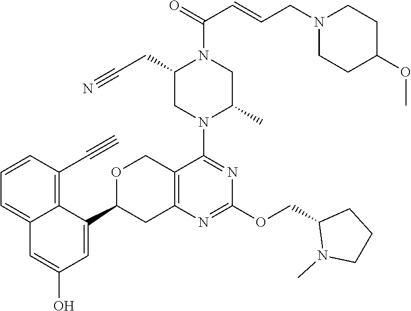 [SMILES: C#Cc1cccc2cc(O)cc([C@@H]3Cc4nc(OC[C@@H]5CCCN5C)nc(N5C[C@H](CC#N)N(C(=O)/C=C/CN6CCC(OC)CC6)C[C@@H]5C)c4CO3)c12] | 2-((2S,5S)-4-((S)-7-(8- ethynyl-3-hydroxynaph- thalen-1-yl)-2-(((S)-1-meth- ylpyrrolidin-2-yl)methoxy)- 7,8-dihydro-5H-pyrano[4,3- d]pyrimidin-4-yl)-1-((E)-4- (4-methoxypiperidin-1-yl)- but-2-enoyl)-5-methyl- piperazin-2-yl)acetonitrile | 734 |
| Ex. 16 | 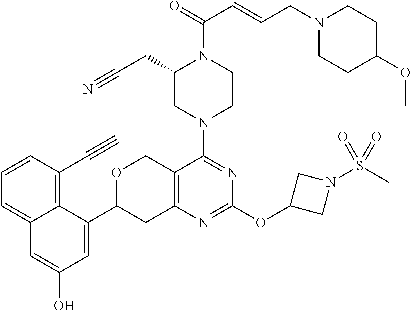 [SMILES: C#Cc1cccc2cc(O)cc(C3Cc4nc(OC5CN(S(C)(=O)=O)C5)nc(N5CCN(C(=O)/C=C/CN6CCC(OC)CC6)[C@@H](CC#N)C5)c4CO3)c12] | 2-((2S)-4-(7-(8-ethynyl-3- hydroxynaphthalen-1-yl)-2- ((1-(methylsulfonyl)azeti- din-3-yl)oxy)-7,8-dihydro- 5H-pyrano[4,3-d]pyrimidin- 4-yl)-1-((E)-4-(4-methoxy- piperidin-1-yl)but-2-enoyl)- piperazin-2-yl)acetonitrile | 756 |
| Ex. 17 | 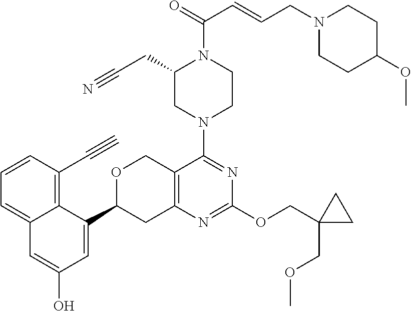 [SMILES: C#Cc1cccc2cc(O)cc([C@@H]3Cc4nc(OCC5(COC)CC5)nc(N5CCN(C(=O)/C=C/CN6CCC(OC)CC6)[C@@H](CC#N)C5)c4CO3)c12] | 2-((S)-4-((S)-7-(8-ethynyl- 3-hydroxynaphthalen-1-yl)- 2-((1-(methoxymethyl)- cyclopropyl)methoxy)-7,8- dihydro-5H-pyrano[4,3-d]- pyrimidin-4-yl)-1-((E)-4-(4- methoxypiperidin-1-yl)but- 2-enoyl)piperazin-2-yl)- acetonitrile | 721 |
| Ex. 18 | 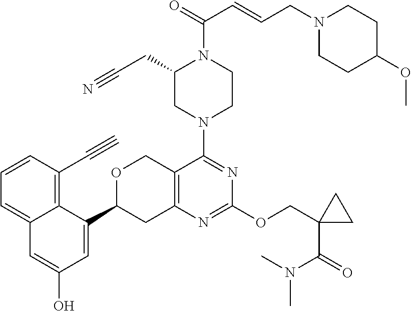 [SMILES: C#Cc1cccc2cc(O)cc([C@@H]3Cc4nc(OCC5(C(=O)N(C)C)CC5)nc(N5CCN(C(=O)/C=C/CN6CCC(OC)CC6)[C@@H](CC#N)C5)c4CO3)c12] | 1-((((S)-4-((S)-3-(cyano- methyl)-4-((E)-4-(4-meth- oxypiperidin-1-yl)but-2- enoyl)piperazin-1-yl)-7-(8- ethynyl-3-hydroxynaph- thalen-1-yl)-7,8-dihydro- 5H-pyrano[4,3-d]pyrimidin- 2-yl)oxy)methyl)-N,N- dimethylcyclopropane-1- carboxamide | 748 |
| Ex. 19 | 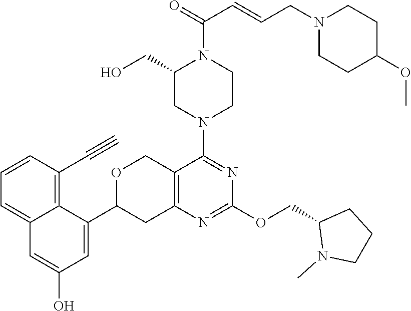 [SMILES: C#Cc1cccc2cc(O)cc(C3Cc4nc(OC[C@@H]5CCCN5C)nc(N5CCN(C(=O)/C=C/CN6CCC(OC)CC6)[C@@H](CO)C5)c4CO3)c12] | (E)-1-((2R)-4-(7-(8-ethynyl- 3-hydroxynaphthalen-1-yl)- 2-(((S)-1-methylpyrrolidin- 2-yl)methoxy)-7,8-dihydro- 5H-pyrano[4,3-d]pyrimidin- 4-yl)-2-(hydroxymethyl)- piperazin-1-yl)-4-(4-meth- oxypiperidin-1-yl)but-2-en- 1-one | 711 |

### Example 20: (S,E)-2-(1-(4-(4-methoxypiperidin-1-yl)but-2-enoyl)-4-(2′-((1-(morpholinomethyl)cyclopropyl)methoxy)-1,3,5′,8′-tetrahydrospiro[indene-2,7′-pyrano[4,3-d]pyrimidin]-4′-yl)piperazin-2-yl)acetonitrile (Ex. 20)

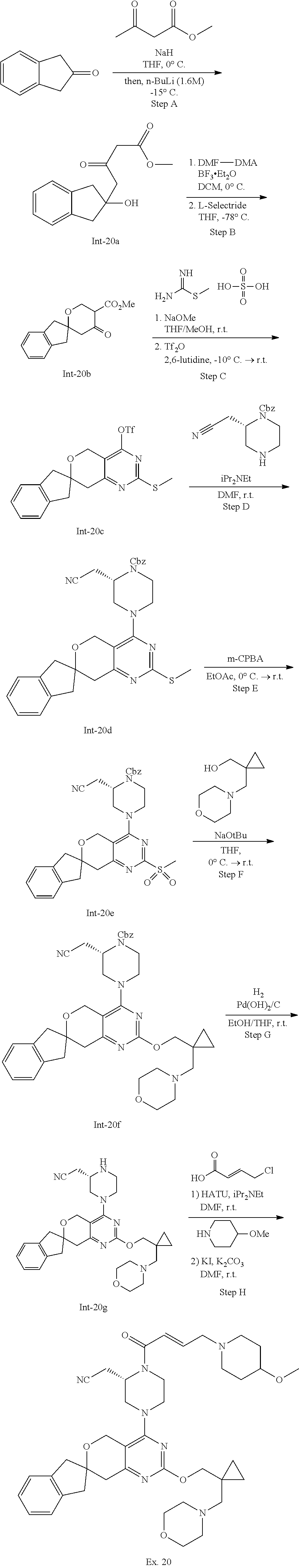

### Step A: Methyl 4-(2-hydroxy-2,3-dihydro-1H-inden-2-yl)-3-oxobutanoate (Int-20a)

To a solution of methyl acetoacetate (2.9 mL, 27 mmol) in THF (35 mL) was added sodium hydride (1.1 g, 27 mmol, 60% dispersion in paraffin liquid) at 0° C., and the mixture was stirred for 30 min at 0° C. To the mixture was dropwise added n-butyl lithium (17 mL, 27 mmol, 1.6 M in hexane) at −15° C., and the mixture was stirred for an hour at the same temperature. To the mixture was added 2-indanone (2.0 g, 15 mmol) at −15° C., and the mixture was stirred for 30 min at the same temperature. To the mixture was added saturated aqueous NH₄Cl solution, followed by dilution with EtOAc. The organic layer was separated and washed with H₂O and concentrated. The residue was purified by column chromatography on silica gel (gradient elution, EtOAc/Hexane) to give the title compound (Int-20a).

### Step B: Methyl 4′-oxo-1,3,3′,4′,5′,6′-hexahydrospiro[indene-2,2′-pyran]-5′-carboxylate (Int-20b)

To a solution of methyl 4-(2-hydroxy-2,3-dihydro-1H-inden-2-yl)-3-oxobutanoate (Int-20a) (1090 mg, 4.4 mmol) in dichloromethane (20 mL) was added N,N-dimethylformamide dimethyl acetal (0.70 mL, 5.3 mmol) at room temperature, and the mixture was stirred overnight. To the mixture was added boron trifluoride-ethyl ether complex (0.67 mL, 5.3 mmol) at 0° C., and then the mixture was diluted with EtOAc. After removal of dichloromethane under vacuo, saturated aqueous NaHCO₃ solution was added to the mixture, and the organic layer was separated and washed with H₂O and concentrated. The residue was used for the next reaction without purification.

To a solution of the crude residue in THF (10 mL) was added L-Selectride solution (4.4 mL, 4.4 mmol, 1M solution in THF) dropwise at −78° C., and the mixture was stirred for 30 minutes at the same temperature. Additional L-Selectride solution (0.5 mL) was added, and then saturated aqueous NH₄Cl solution was added. The mixture was extracted with EtOAc, and the organic layer was separated and washed with H₂O and concentrated. The residue was purified by column chromatography on silica gel (gradient elution, EtOAc/Hexane) to give the title compound (Int-20b).

### Step C: 2′-(methylthio)-1,3,5′,8′-tetrahydrospiro[indene-2,7′-pyrano[4,3-d]pyrimidin]-4′-yl trifluoromethanesulfonate (Int-20c)

To a solution of methyl 4′-oxo-1,3,3′,4′,5′,6′-hexahydrospiro[indene-2,2′-pyran]-5′-carboxylate (Int-20b) (890 mg, 3.4 mmol) and S-methylisothiourea sulfate (fine powdered, 1.0 g, 6.8 mmol) in MeOH (30 mL) and THF (10 mL) was added sodium methoxide (923 mg, 17 mmol) at room temperature, and the mixture was stirred for 3 hours at the same temperature. The solvent was concentrated in vacuo, and dichloromethane was added to the residue The mixture was washed with H₂O and concentrated to give crude compound. The crude compound was used for the next step without purification.

To a solution of crude compound (780 mg) in 2,6-lutidine (15 mL) was added trifluoromethanesulfonic anhydride (2.3 mL, 14 mmol) dropwise at −10° C., and the mixture was stirred for 10 min at room temperature. After cooling to −10° C., additional trifluoromethanesulfonic anhydride (1.7 mL, 7 mmol) was added, and the mixture was allowed to warm to room temperature. After the reaction was completed, to the mixture was added EtOAc and aqueous HCl solution (1M), followed by extraction with EtOAc. The organic layer was separated and washed with aqueous HCl solution (1M) and H₂O and concentrated. The residue was purified by column chromatography on silica gel (gradient elution, EtOAc/Hexane) to give the title compound (Int-20c). ESI-MS m/z 433 (M+H)⁺.

### Step D: Benzyl (S)-2-(cyanomethyl)-4-(2′-(methylthio)-1,3,5′,8′-tetrahydrospiro[indene-2,7′-pyrano[4,3-d]pyrimidin]-4′-yl)piperazine-1-carboxylate (Int-20d)

To a solution of 2′-(methylthio)-1,3,5′,8′-tetrahydrospiro[indene-2,7′-pyrano[4,3-d]pyrimidin]-4′-yl trifluoromethanesulfonate (Int-20c) (300 mg, 0.69 mmol) in DMA (3 mL) was added benzyl (S)-2-(cyanomethyl)piperazine-1-carboxylate (200 mg, 1.0 mmol) and N,N-diisopropylethylamine (0.47 mL, 2.8 mmol) at room temperature. After stirring at the same temperature for 2 hours, the reaction mixture was diluted with EtOAc and saturated aqueous NH₄Cl solution. The organic layer was separated and washed with H₂O and concentrated. The residue was purified by column chromatography on silica gel (gradient elution, EtOAc/Hexane) to give the title compound (Int-20d). ESI-MS m/z 542 (M+H)⁺.

### Step E: Benzyl (S)-2-(cyanomethyl)-4-(2′-(methylsulfonyl)-1,3,5′,8′-tetrahydrospiro[indene-2,7′-pyrano[4,3-d]pyrimidin]-4′-yl)piperazine-1-carboxylate (Int-20e)

To a solution of benzyl (S)-2-(cyanomethyl)-4-(2′-(methylthio)-1,3,5′,8′-tetrahydrospiro[indene-2,7′-pyrano[4,3-d]pyrimidin]-4′-yl)piperazine-1-carboxylate (Int-20d) (330 mg, 0.61 mmol) in EtOAc (10 mL) was added m-chloroperoxybenzoic acid (231 mg, 1.3 mmol, with abt. 25% water) at 0° C., and the mixture was stirred for 2 hours at room temperature. To the mixture was added aqueous NaHCO₃ solution, and the organic layer was separated and washed with H₂O and concentrated. The residue was purified by column chromatography on silica gel (gradient elution, EtOAc/Hexane) to give the title compound (Int-20e). ESI-MS m/z 574 (M+H)⁺.

### Step F: Benzyl (S)-2-(cyanomethyl)-4-(2′-((1-(morpholinomethyl)cyclopropyl)methoxy)-1,3,5′,8′-tetrahydrospiro[indene-2,7′-pyrano[4,3-d]pyrimidin]-4′-yl)piperazine-1-carboxylate (Int-20f)

To a solution of benzyl (S)-2-(cyanomethyl)-4-(2′-(methylsulfonyl)-1,3,5′,8′-tetrahydrospiro[indene-2,7′-pyrano[4,3-d]pyrimidin]-4′-yl)piperazine-1-carboxylate (Int-20e) (300 mg, 0.52 mmol) and (1-(morpholinomethyl)cyclopropyl)methanol (210 mg, 1.0 mmol) in THF (5 mL) was added potassium tert-butoxide (0.68 mL, 0.68 mmol, 1M solution in THF) at 0° C., and the mixture was stirred for an hour at the same temperature. To the mixture was added EtOAc and water, and the organic layer was separated and washed with H₂O and concentrated. The residue was purified by column chromatography on NH silica gel (gradient elution, EtOAc/Hexane) to give the title compound (Int-20f). ESI-MS m/z 665 (M+H)⁺.

### Step G: (S)-2-(4-(2′-((1-(morpholinomethyl)cyclopropyl)methoxy)-1,3,5′,8′-tetrahydrospiro[indene-2,7′-pyrano[4,3-d]pyrimidin]-4′-yl)piperazin-2-yl)acetonitrile (Int-20g)

To a solution of benzyl (S)-2-(cyanomethyl)-4-(2′-((1-(morpholinomethyl)cyclopropyl)methoxy)-1,3,5′,8′-tetrahydrospiro[indene-2,7′-pyrano[4,3-d]pyrimidin]-4′-yl)piperazine-1-carboxylate (Int-20f) (0.26 g, 0.39 mmol) in EtOH (2 mL) and THF (2 mL) was added Pd(OH)₂/C (200 mg, Pd 10%, wetted with ca. 50% water) at room temperature, and the mixture was stirred at the same temperature under H₂ atmosphere. After stirring for 30 min, the vessel was degassed and purged with N₂, and the mixture was filtered and concentrated in vacuo. The residue was purified by column chromatography on amine-functionalized silica gel (gradient elution, MeOH/EtOAc) to give the title compound (Int-20g). ESI-MS m/z 531 (M+H)⁺.

### Step H: (S,E)-2-(1-(4-(4-methoxypiperidin-1-yl)but-2-enoyl)-4-(2′-((1-(morpholinomethyl)cyclopropyl)methoxy)-1,3,5′,8′-tetrahydrospiro[indene-2,7′-pyrano[4,3-d]pyrimidin]-4′-yl)piperazin-2-yl)acetonitrile (Ex. 20)

To a solution of (S)-2-(4-(2′-((1-(morpholinomethyl)cyclopropyl)methoxy)-1,3,5′,8′-tetrahydrospiro[indene-2,7′-pyrano[4,3-d]pyrimidin]-4′-yl)piperazin-2-yl)acetonitrile (Int-20g) (39 mg, 0.073 mmol) in DMF (1 mL) was added 1-[Bis(dimethylamino)methylene]-1H-1,2,3-triazolo[4,5-b]pyridinium 3-oxide hexafluorophosphate (33 mg, 0.088 mmol), (E)-4-chlorobut-2-enoic acid (11 mg, 0.088 mmol) and N,N-diisopropylethylamine (40 uL, 0.22 mmol) at room temperature. After stirring for 30 min, the reaction mixture was diluted with EtOAc, and the mixture was washed with water. The organic layer was dried over sodium sulfate. The dried solution was filtered, and the filtrate was concentrated in vacuo. The crude compound was used in the next step without further purification.

To a solution of crude compound in DMF (1 mL) was added 4-methoxypiperidine (40 mg, 0.35 mmol), potassium iodide (40 mg, 0.24 mmol) and potassium carbonate (40 mg, 0.29 mmol) at room temperature. The reaction mixture was heated at 50° C. for 3 h. After the reaction was completed, the reaction mixture was diluted with EtOAc, and the mixture was washed with water. The organic layer was dried over sodium sulfate. The dried solution was filtered, and the filtrate was concentrated in vacuo. The residue was purified by column chromatography on amine-functionalized silica gel (gradient elution, MeOH/EtOAc) to give the title compound (Ex. 20). 1H-NMR (400 MHz, DMSO-d₆) δ 7.24-7.19 (m, 2H), 7.19-7.14 (m, 2H) 6.69-6.65 (m, 2H), 4.70 (s, 2H), 4.15-4.09 (m, 2H), 3.89-3.86 (m, 1H), 3.77-3.74 (m, 1H), 3.55-3.50 (m, 3H), 3.40-3.00 (m, 20H), 3.00-2.82 (m, 4H), 2.69-2.61 (m, 2H), 2.31-2.24 (m, 2H), 2.12-2.06 (m, 2H), 1.86-1.79 (m, 2H), 1.47-1.39 (m, 2H) 0.59-0.56 (m, 2H), 0.40-0.37 (m, 2H). ESI-MS m/z 712 (M+H)⁺.

### Example 21: 2-((2S)-4-(7-bromo-2′-((1-(morpholinomethyl)cyclopropyl)methoxy)-2,3,5′,8′-tetrahydrospiro[indene-1,7′-pyrano[4,3-d]pyrimidin]-4′-yl)-1-((E)-4-(4-methoxypiperidin-1-yl)but-2-enoyl)piperazin-2-yl)acetonitrile (Ex. 21)

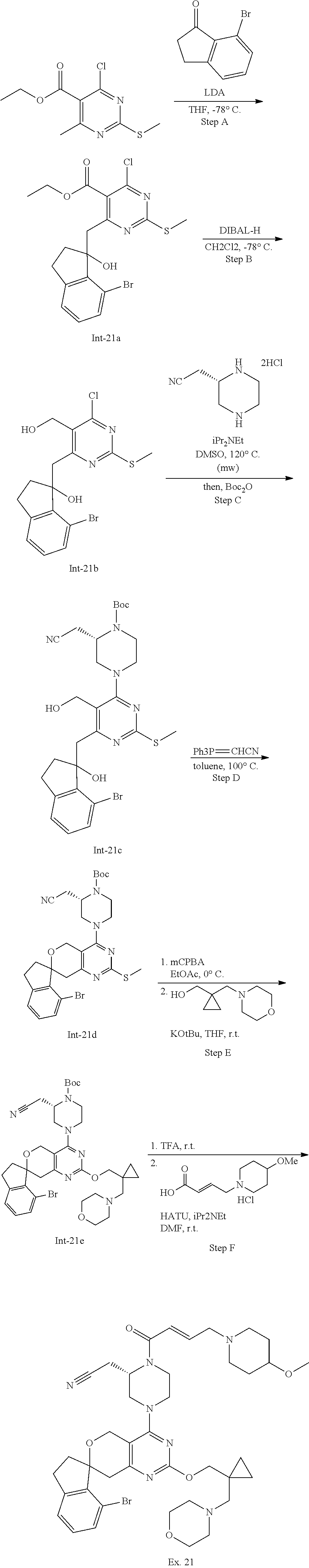

### Step A: ethyl 4-((7-bromo-1-hydroxy-2,3-dihydro-1H-inden-1-yl)methyl)-6-chloro-2-(methylthio)pyrimidine-5-carboxylate (Int-21a)

BuLi (1.54 M in hexane, 9.06 mL, 14.0 mmol) was slowly added at −78° C. to diisopropylamine (2.24 mL, 16.0 mmol) in THF (20 mL), and then a solution of ethyl 4-chloro-6-methyl-2-(methylthio)pyrimidine-5-carboxylate (2.46 g, 9.97 mmol) in THF was added thereto. After stirring at −78° C. for 30 min, a solution of 7-bromo-2,3-dihydro-1H-inden-1-one (2.53 g, 12.0 mmol) in THF (5 mL) was added slowly thereto. The reaction mixture was stirred at −78° C. for 10 min, then quenched by addition of a saturated aqueous ammonium chloride solution and warmed to room temperature. The resulting mixture was extracted with ethyl acetate. The organic layer was washed with brine, dried over sodium sulfate and evaporated under reduced pressure. The residue was purified by column chromatography on silica gel (hexane-EtOAc) to give ethyl 4-((7-bromo-1-hydroxy-2,3-dihydro-1H-inden-1-yl)methyl)-6-chloro-2-(methylthio)pyrimidine-5-carboxylate (Int-21a). ESI-MS m/z 459 (M+H)⁺.

### Step B: 7-bromo-1-((6-chloro-5-(hydroxymethyl)-2-(methylthio)pyrimidin-4-yl)methyl)-2,3-dihydro-1H-inden-1-ol (Int-21b)

To the mixture of ethyl 4-((7-bromo-1-hydroxy-2,3-dihydro-1H-inden-1-yl)methyl)-6-chloro-2-(methylthio)pyrimidine-5-carboxylate (Int-21a) (2.34 g, 5.11 mmol) in dichloromethane (30 mL) was slowly added DIBAL-H (1M in hexane, 15.3 mL, 15.3 mmol) at −78° C., followed by stirring for 40 min. An aqueous Rochelle salt solution (8 g/80 mL) was added thereto and warmed to room temperature. After stirring for 3 hrs, the resulting mixture was extracted with ethyl acetate. The organic layer was washed with saturated aqueous NaHCO₃ and brine, dried over sodium sulfate and evaporated under reduced pressure. The residue was purified by column chromatography on silica gel (hexane-AcOEt) to give 7-bromo-1-((6-chloro-5-(hydroxymethyl)-2-(methylthio)pyrimidin-4-yl)methyl)-2,3-dihydro-1H-inden-1-ol (Int-21b). ESI-MS m/z 417 (M+H)⁺.

### Step C: tert-butyl (2S)-4-(6-((7-bromo-1-hydroxy-2,3-dihydro-1H-inden-1-yl)methyl)-5-(hydroxymethyl)-2-(methylthio)pyrimidin-4-yl)-2-(cyanomethyl)piperazine-1-carboxylate (Int-21c)

The mixture of 7-bromo-1-((6-chloro-5-(hydroxymethyl)-2-(methylthio)pyrimidin-4-yl)methyl)-2,3-dihydro-1H-inden-1-ol (Int-21b) (300 mg, 0.722 mmol), 2-[(2S)-piperazin-2-yl]acetonitrile; dihydrochloride (186 mg, 0.938 mmol), and N,N-diisopropylethylamine (0.598 mL, 3.61 mmol) in DMSO (3 mL) was reacted by microwave apparatus at 120° C. for 2 hrs. To the resulting mixture was added Boc (394 mg, 1.80 mmol) at room temperature followed by stirring for 3 hrs. The resulting mixture was diluted with ethyl acetate and saturated aqueous NaHCO₃. The organic layer was separated and washed with water and brine, dried over sodium sulfate and evaporated under reduced pressure. The residue was purified by column chromatography on silica gel (hexane-AcOEt) to give tert-butyl (2S)-4-(6-((7-bromo-1-hydroxy-2,3-dihydro-1H-inden-1-yl)methyl)-5-(hydroxymethyl)-2-(methylthio)pyrimidin-4-yl)-2-(cyanomethyl)piperazine-1-carboxylate (Int-21c). ESI-MS m/z 604 (M+H)⁺.

### Step D: tert-butyl (2S)-4-(7-bromo-2′-(methylthio)-2,3,5′,8′-tetrahydrospiro[indene-1,7′-pyrano[4,3-d]pyrimidin]-4′-yl)-2-(cyanomethyl)piperazine-1-carboxylate (Int-21d)

To the solution of tert-butyl (2S)-4-(6-((7-bromo-1-hydroxy-2,3-dihydro-1H-inden-1-yl)methyl)-5-(hydroxymethyl)-2-(methylthio)pyrimidin-4-yl)-2-(cyanomethyl)piperazine-1-carboxylate (Int-21c) (250 mg, 0.44 mmol) in toluene (5 mL) was added (triphenylphosphoranylidene)acetonitrile (562 uL, 2.16 mmol). The mixture was stirred at 100° C. for 20 hrs. Methanol was added to the resulting mixture and evaporated under reduced pressure. The residue was purified by column chromatography on silica gel (hexane-AcOEt) to give tert-butyl (2S)-4-(7-bromo-2′-(methylthio)-2,3,5′,8′-tetrahydrospiro[indene-1,7′-pyrano[4,3-d]pyrimidin]-4′-yl)-2-(cyanomethyl)piperazine-1-carboxylate (Int-21d). ESI-MS m/z 588 (M+H)⁺

### Step E: tert-butyl (2S)-4-(7-bromo-2′-((1-(morpholinomethyl)cyclopropyl)methoxy)-2,3,5′,8′-tetrahydrospiro[indene-1,7′-pyrano[4,3-d]pyrimidin]-4′-yl)-2-(cyanomethyl)piperazine-1-carboxylate (Int-21e)

mCPBA (approximately 75% purity, 25.9 mg) was added at 0° C. to a solution of tert-butyl (2S)-4-(7-bromo-2′-(methylthio)-2,3,5′,8′-tetrahydrospiro[indene-1,7′-pyrano[4,3-d]pyrimidin]-4′-yl)-2-(cyanomethyl)piperazine-1-carboxylate (Int-21d) (55 mg, 0.0938 mmol) in ethyl acetate (2 mL). After stirring at 0° C. for 60 min, the resulting mixture was diluted with ethyl acetate and saturated aqueous NaHCO₃. The organic layer was separated and washed with water and brine, dried over sodium sulfate and evaporated under reduced pressure. The residue was diluted with THF (2 mL), and [1-(morpholinomethyl)cyclopropyl]methanol (64.2 mg, 0.375 mmol) and potassium tert-butoxide (1M in THF, 0.141 mL, 0.141 mmol) was added thereto. The resulting mixture was diluted with ethyl acetate and saturated aqueous NaHCO₃. The organic layer was separated and washed with water and brine, dried over sodium sulfate and evaporated under reduced pressure. The residue was purified by column chromatography on amino silica gel (hexane-AcOEt) to give tert-butyl (2S)-4-(7-bromo-2′-((1-(morpholinomethyl)cyclopropyl)methoxy)-2,3,5′,8′-tetrahydrospiro[indene-1,7′-pyrano[4,3-d]pyrimidin]-4′-yl)-2-(cyanomethyl)piperazine-1-carboxylate (Int-21e). ESI-MS m/z 709, 711 (M+H)⁺.

### Step F: 2-((2S)-4-(7-bromo-2′-((1-(morpholinomethyl)cyclopropyl)methoxy)-2,3,5′,8′-tetrahydrospiro[indene-1,7′-pyrano[4,3-d]pyrimidin]-4′-yl)-1-((E)-4-(4-methoxypiperidin-1-yl)but-2-enoyl)piperazin-2-yl)acetonitrile (Ex. 21)

tert-butyl (2S)-4-(7-bromo-2′-((1-(morpholinomethyl)cyclopropyl)methoxy)-2,3,5′,8′-tetrahydrospiro[indene-1,7′-pyrano[4,3-d]pyrimidin]-4′-yl)-2-(cyanomethyl)piperazine-1-carboxylate (Int-21e) (38 mg, 0.0535 mmol) was treated by TFA (0.5 mL) at room temperature for 5 min. The mixture was concentrated, and DMF (2 mL), (E)-4-(4-methoxypiperidin-1-yl)but-2-enoic acid hydrochloride (25.2 mL), N,N-diisopropylethylamine (71 uL), and HATU (30.5 mg, 0.0803 mmol) were added thereto. The resulting mixture was stirred at room temperature for 30 min. The resulting mixture was diluted with ethyl acetate and saturated aqueous NaHCO₃. The organic layer was separated and washed with water and brine, dried over sodium sulfate and evaporated under reduced pressure. The residue was purified by preparative reversed-phase HPLC (water:acetonitrile (0.1% formic acid)) to give the title compound (Ex. 21). 1H-NMR (400 MHz, CDCl₃) δ 7.43 (d, J=7.8 Hz, 1H), 7.23-7.20 (m, 1H), 7.14 (t, J=7.6 Hz, 1H), 7.00-6.93 (m, 1H), 6.43 (d, J=13.5 Hz, 1H), 5.07-4.75 (m, 3H), 4.27-4.19 (m, 2H), 4.00-3.93 (m, 2H), 3.67 (t, J=4.0 Hz, 4H), 3.51-3.44 (m, 2H), 3.34 (s, 3H), 3.29-3.21 (m, 2H), 3.16 (d, J=5.0 Hz, 2H), 3.12-3.01 (m, 2H), 2.96-2.73 (m, 6H), 2.47 (s, 4H), 2.40 (d, J=5.8 Hz, 2H), 2.36-2.17 (m, 4H), 1.93-1.90 (m, 2H), 1.68-1.58 (m, 3H), 0.65 (t, J=6.4 Hz, 2H), 0.44 (t, J=5.1 Hz, 2H) ESI-MS m/z 790, 792 (M+H)⁺.

The example in the table below was prepared in a similar manner to Ex. 21 above.

| Ex. | Structure | Compound Name | [M + H]⁺ |
|---|---|---|---|
| Ex. 22 | 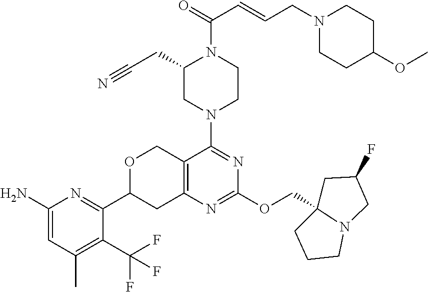 | 2-((2S)-4-(7-(6-amino-4- methyl-3-(trifluoromethyl)- pyridin-2-yl)-2-(((2R,7aS)- 2-fluorotetrahydro-1H-pyr- rolizin-7a(5H)-yl)methoxy)- 7,8-dihydro-5H-pyrano[4,3- d]pyrimidin-4-yl)-1-((E)-4- (4-methoxypiperidin-1-yl)- but-2-enoyl)piperazin-2-yl)- acetonitrile | 772 |

### Example 23: 2-((2S)-4-(7-(2-amino-5,7-difluoroquinolin-8-yl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-1-((E)-4-(4-methoxypiperidin-1-yl)but-2-enoyl)piperazin-2-yl)acetonitrile (Ex. 23)

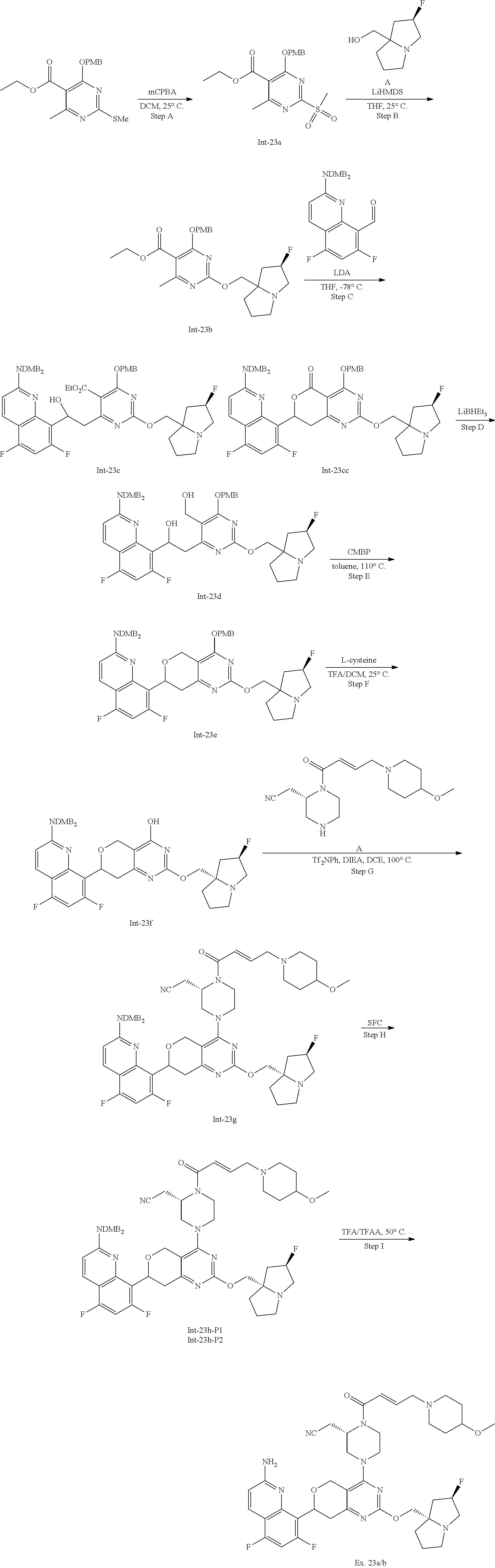

### Step A: ethyl 4-((4-methoxybenzyl)oxy)-6-methyl-2-(methylsulfonyl)pyrimidine-5-carboxylate (Int-23a)

To a stirred solution of ethyl 4-((4-methoxybenzyl)oxy)-6-methyl-2-(methylthio)pyrimidine-5-carboxylate (4 g, 11.48 mmol) in DCM (60 mL) was added m-CPBA (5.45 g, 25.3 mmol, 85%) at 25° C. under N₂, the reaction was stirred at 25° C. for 2 h. The mixture was added NaHCO₃ (100 mL), extracted with EtOAc (30 mL×3), the organic layers were washed with sat. NaCl (50 mL), dried over Na₂SO₄ and concentrated. The residue was purified by flash silica gel chromatography (ISCO®; Agela® Flash Column Silica-CS (40 g), Eluent of 0˜16% Ethyl acetate/Petroleum ether gradient @ 30 mL/min) to give ethyl 4-((4-methoxybenzyl)oxy)-6-methyl-2-(methylsulfonyl)pyrimidine-5-carboxylate (Int-23a). MS (ESI) [M+H]+ m/z 381.

### Step B: ethyl 2-(((2R)-2-fluorotetrahydro-1H-pyrrolizin-7a(5H)-yl)methoxy)-4-((4-methoxybenzyl)oxy)-6-methylpyrimidine-5-carboxylate (Int-23b)

To a stirred solution of ((2R)-2-fluorotetrahydro-1H-pyrrolizin-7a(5H)-yl)methanol (1.883 g, 11.83 mmol) in dry THF (50 mL) was added LiHMDS (11.83 mL, 11.83 mmol, 1M) at 0° C. under N₂, the reaction was stirred at 25° C. for 0.5 h. Then ethyl 4-((4-methoxybenzyl)oxy)-6-methyl-2-(methylsulfonyl)pyrimidine-5-carboxylate (Int-23a) (3 g, 7.89 mmol) was added, the reaction was stirred at 25° C. for 0.5 h. The mixture was quenched with water (30 mL), extracted with EtOAc (20 mL×3), the organic layers were washed with sat. NaCl (20 mL), dried over Na₂SO₄ and concentrated. The residue was purified by flash silica gel chromatography (ISCO®; Agela® Flash Column Silica-CS (40 g), Eluent of 0˜36% Ethyl acetate/Petroleum ether gradient @ 30 mL/min) to give ethyl 2-(((2R)-2-fluorotetrahydro-1H-pyrrolizin-7a(5H)-yl)methoxy)-4-((4-methoxybenzyl)oxy)-6-methylpyrimidine-5-carboxylate (Int-23b). MS (ESI) m/z 460.

### Step C: ethyl 4-(2-(2-(bis(2,4-dimethoxybenzyl)amino)-5,7-difluoroquinolin-8-yl)-2-hydroxyethyl)-2-(((2R)-2-fluorotetrahydro-1H-pyrrolizin-7a(5H)-yl)methoxy)-6-((4-methoxybenzyl)oxy)pyrimidine-5-carboxylate (Int-23c) and 7-(2-(bis(2,4-dimethoxybenzyl)amino)-5,7-difluoroquinolin-8-yl)-2-(((2S)-2-fluorotetrahydro-1H-pyrrolizin-7a(5H)-yl)methoxy)-4-((4-methoxybenzyl)oxy)-7,8-dihydro-5H-pyrano[4,3-d]pyrimidin-5-one (Int-23cc)

To a stirred solution of ethyl 2-(((2R)-2-fluorotetrahydro-1H-pyrrolizin-7a(5H)-yl)methoxy)-4-((4-methoxybenzyl)oxy)-6-methylpyrimidine-5-carboxylate (Int-23b) (1 g, 2.176 mmol) in dry THF (20 mL) was added LDA (3.26 mL, 3.26 mmol) at −78° C. under N₂. The reaction was stirred at −78° C. for 0.5 h, then 2-(bis(2,4-dimethoxybenzyl)amino)-5,7-difluoroquinoline-8-carbaldehyde (1.771 g, 3.48 mmol) in THF (5 mL) was added at −78° C. After the addition was finished, the reaction was stirred at −78° C. for 1 h under N₂. The mixture was quenched with NH₄Cl (30 mL), extracted with EtOAc (30 mL×3), the organic layers were washed with sat. NaCl (50 mL), dried over Na₂SO₄ and concentrated. The residue was purified by flash silica gel chromatography (ISCO®; Agela® Flash Column Silica-CS (12 g), using an eluent of 0-30% Ethyl acetate/Petroleum ether gradient @ 30 mL/min) to give the 1:1 mixture of ethyl 4-(2-(2-(bis(2,4-dimethoxybenzyl)amino)-5,7-difluoroquinolin-8-yl)-2-hydroxyethyl)-2-(((2R)-2-fluorotetrahydro-1H-pyrrolizin-7a(5H)-yl)methoxy)-6-((4-methoxybenzyl)oxy)pyrimidine-5-carboxylate (Int-23c) and 7-(2-(bis(2,4-dimethoxybenzyl)amino)-5,7-difluoroquinolin-8-yl)-2-(((2S)-2-fluorotetrahydro-1H-pyrrolizin-7a(5H)-yl)methoxy)-4-((4-methoxybenzyl)oxy)-7,8-dihydro-5H-pyrano[4,3-d]pyrimidin-5-one (Int-23cc). MS (ESI) [M+H]+: m/z 923, 969.

### Step D: 1-(2-(bis(2,4-dimethoxybenzyl)amino)-5,7-difluoroquinolin-8-yl)-2-(2-(((2R)-2-fluorotetrahydro-1H-pyrrolizin-7a(5H)-yl)methoxy)-5-(hydroxymethyl)-6-((4-methoxybenzyl)oxy)pyrimidin-4-yl)ethan-1-ol (Int-23d)

To a stirred solution of ethyl 4-(2-(2-(bis(2,4-dimethoxybenzyl)amino)-5,7-difluoroquinolin-8-yl)-2-hydroxyethyl)-2-(((2R)-2-fluorotetrahydro-1H-pyrrolizin-7a(5H)-yl)methoxy)-6-((4-methoxybenzyl)oxy)pyrimidine-5-carboxylate (Int-23c) and 7-(2-(bis(2,4-dimethoxybenzyl)amino)-5,7-difluoroquinolin-8-yl)-2-(((2S)-2-fluorotetrahydro-1H-pyrrolizin-7a(5H)-yl)methoxy)-4-((4-methoxybenzyl)oxy)-7,8-dihydro-5H-pyrano[4,3-d]pyrimidin-5-one (Int-23cc) (1 g, 0.529 mmol) in THF (15 mL) was added lithium triethylhydroborate (5.29 mL, 5.29 mmol) at 0° C. After the addition was finished, the reaction was stirred at 0° C. for 1 h. The mixture was quenched with water (20 mL), extracted with EtOAc (15 mL×3), the organic layers were washed with sat. NaCl (20 mL), dried over Na₂SO₄ and concentrated. The residue was purified by flash silica gel chromatography (ISCO®; Agela® Flash Column Silica-CS (12 g), Eluent of 0˜36% Ethyl acetate/Petroleum ether gradient @ 30 mL/min) to give 1-(2-(bis(2,4-dimethoxybenzyl)amino)-5,7-difluoroquinolin-8-yl)-2-(2-(((2R)-2-fluorotetrahydro-1H-pyrrolizin-7a(5H)-yl)methoxy)-5-(hydroxymethyl)-6-((4-methoxybenzyl)oxy)pyrimidin-4-yl)ethan-1-ol (Int-23d). MS (ESI) [M+H]+: m/z 926.

### Step E: N,N-bis(2,4-dimethoxybenzyl)-5,7-difluoro-8-(2-(((2R)-2-fluorotetrahydro-1H-pyrrolizin-7a(5H)-yl)methoxy)-4-((4-methoxybenzyl)oxy)-7,8-dihydro-5H-pyrano[4,3-d]pyrimidin-7-yl)quinolin-2-amine (Int-23e)

To a stirred solution of 1-(2-(bis(2,4-dimethoxybenzyl)amino)-5,7-difluoroquinolin-8-yl)-2-(2-(((2R)-2-fluorotetrahydro-1H-pyrrolizin-7a(5H)-yl)methoxy)-5-(hydroxymethyl)-6-((4-methoxybenzyl)oxy)pyrimidin-4-yl)ethan-1-ol (Int-23d) (400 mg, 0.432 mmol) in toluene (10 mL) was added (tributylphosphoranylidene)acetonitrile (521 mg, 2.160 mmol) at 25° C. under N₂, the reaction was stirred at 100° C. for 2 h. The mixture was concentrated and purified by flash silica gel chromatography (ISCO®; Agela® Flash Column Silica-CS (12 g), Eluent of 0˜80% Ethyl acetate/Petroleum ether gradient @ 30 mL/min) to give N,N-bis(2,4-dimethoxybenzyl)-5,7-difluoro-8-(2-(((2R)-2-fluorotetrahydro-1H-pyrrolizin-7a(5H)-yl)methoxy)-4-((4-methoxybenzyl)oxy)-7,8-dihydro-5H-pyrano[4,3-d]pyrimidin-7-yl)quinolin-2-amine (Int-23e). MS (ESI) [M+H]+: m/z 909.

### Step F: 7-(2-(bis(2,4-dimethoxybenzyl)amino)-5,7-difluoroquinolin-8-yl)-2-(((2R)-2-fluorotetrahydro-1H-pyrrolizin-7a(5H)-yl)methoxy)-7,8-dihydro-5H-pyrano[4,3-d]pyrimidin-4-ol (Int-23f)

To a stirred solution of N,N-bis(2,4-dimethoxybenzyl)-5,7-difluoro-8-(2-(((2R)-2-fluorotetrahydro-1H-pyrrolizin-7a(5H)-yl)methoxy)-4-((4-methoxybenzyl)oxy)-7,8-dihydro-5H-pyrano[4,3-d]pyrimidin-7-yl)quinolin-2-amine (Int-23e) (190 mg, 0.209 mmol) in TFA/DCM=4:1 (2 mL) was added 1-cysteine hydrochloride (132 mg, 0.837 mmol) at 25° C. After the addition was finished, the reaction was stirred at 25° C. for 2 h. The crude product was purified by prep-TLC (EtOAc/MeOH 10:1 as eluent) to give 7-(2-(bis(2,4-dimethoxybenzyl)amino)-5,7-difluoroquinolin-8-yl)-2-(((2R)-2-fluorotetrahydro-1H-pyrrolizin-7a(5H)-yl)methoxy)-7,8-dihydro-5H-pyrano[4,3-d]pyrimidin-4-ol (Int-23f). MS (ESI) [M+H]⁺ m/z: 788.

### Step G: 2-((2S)-4-(7-(2-(bis(2,4-dimethoxybenzyl)amino)-5,7-difluoroquinolin-8-yl)-2-(((2R)-2-fluorotetrahydro-1H-pyrrolizin-7a(5H)-yl)methoxy)-7,8-dihydro-5H-pyrano[4,3-d]pyrimidin-4-yl)-1-((E)-4-(4-methoxypiperidin-1-yl)but-2-enoyl)piperazin-2-yl)acetonitrile (Int-23g)

To a stirred solution of 7-(2-(bis(2,4-dimethoxybenzyl)amino)-5,7-difluoroquinolin-8-yl)-2-(((2R)-2-fluorotetrahydro-1H-pyrrolizin-7a(5H)-yl)methoxy)-7,8-dihydro-5H-pyrano[4,3-d]pyrimidin-4-ol (Int-23f) (40 mg, 0.038 mmol) and (S,E)-2-(1-(4-(4-methoxypiperidin-1-yl)but-2-enoyl)piperazin-2-yl)acetonitrile (39.2 mg, 0.095 mmol) in DCE (0.5 mL) were added 1,1,1-trifluoro-N-phenyl-N-((trifluoromethyl)sulfonyl)methanesulfonamide (40.8 mg, 0.114 mmol) and DIEA (0.067 mL, 0.381 mmol) at 25° C. After the addition was finished, the reaction was stirred at 100° C. for 3 h under N₂. The mixture was concentrated and purified by reverse phase HPLC (Column Boston Prime C18 150×30 mm×5 um, Condition water (0.05% NH₃H₂O+10 mM NH₄HCO₃)-ACN Begin B 80 End B 100 Gradient Time (min) 10 100% B Hold Time (min) 2 Flow Rate (mL/min) 25 Injections 1) to give 2-((2S)-4-(7-(2-(bis(2,4-dimethoxybenzyl)amino)-5,7-difluoroquinolin-8-yl)-2-(((2R)-2-fluorotetrahydro-1H-pyrrolizin-7a(5H)-yl)methoxy)-7,8-dihydro-5H-pyrano[4,3-d]pyrimidin-4-yl)-1-((E)-4-(4-methoxypiperidin-1-yl)but-2-enoyl)piperazin-2-yl)acetonitrile (Int-23g). MS (ESI) [M+H]⁺ m/z: 539.

### Step H: 2-(4-(7-(2-(bis(2,4-dimethoxybenzyl)amino)-5,7-difluoroquinolin-8-yl)-2-(((2R)-2-fluorotetrahydro-1H-pyrrolizin-7a(5H)-yl)methoxy)-7,8-dihydro-5H-pyrano[4,3-d]pyrimidin-4-yl)-1-((E)-4-(4-methoxypiperidin-1-yl)but-2-enoyl)piperazin-2-yl)acetonitrile (Int-23h-P1 and Int-23h-P2)

2-((2S)-(4-(7-(2-(bis(2,4-dimethoxybenzyl)amino)-5,7-difluoroquinolin-8-yl)-2-(((2R)-2-fluorotetrahydro-1H-pyrrolizin-7a(5H)-yl)methoxy)-7,8-dihydro-5H-pyrano[4,3-d]pyrimidin-4-yl)-1-((E)-4-(4-methoxypiperidin-1-yl)but-2-enoyl)piperazin-2-yl)acetonitrile (Int-23g) (30 mg, 0.028 mmol) was separated by SFC (Column A—Condition 0.1% NH₃H₂O EtOH Begin B 50% End B 50% Gradient Time (min) 100% B Hold Time (min) Flow Rate (mL/min) 80 Injections 50) to give 2-(4-(7-(2-(bis(2,4-dimethoxybenzyl)amino)-5,7-difluoroquinolin-8-yl)-2-(((2R)-2-fluorotetrahydro-1H-pyrrolizin-7a(5H)-yl)methoxy)-7,8-dihydro-5H-pyrano[4,3-d]pyrimidin-4-yl)-1-((E)-4-(4-methoxypiperidin-1-yl)but-2-enoyl)piperazin-2-yl)acetonitrile (the first peak on SFC, Int-23h-P1) and 2-(4-(7-(2-(bis(2,4-dimethoxybenzyl)amino)-5,7-difluoroquinolin-8-yl)-2-(((2R)-2-fluorotetrahydro-1H-pyrrolizin-7a(5H)-yl)methoxy)-7,8-dihydro-5H-pyrano[4,3-d]pyrimidin-4-yl)-1-((E)-4-(4-methoxypiperidin-1-yl)but-2-enoyl)piperazin-2-yl)acetonitrile (the second peak on SFC, Int-23h-P2). MS (ESI) [M+H]⁺ m/z: 539.

### Step I: 2-((2S)-4-(7-(2-amino-5,7-difluoroquinolin-8-yl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-1-((E)-4-(4-methoxypiperidin-1-yl)but-2-enoyl)piperazin-2-yl)acetonitrile (Ex. 23a/b)

To a stirred solution of 2-((2S)-4-(7-(2-(bis(2,4-dimethoxybenzyl)amino)-5,7-difluoroquinolin-8-yl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-1-((E)-4-(4-methoxypiperidin-1-yl)but-2-enoyl)piperazin-2-yl)acetonitrile (13 mg, 0.012 mmol) (Int-23h-P1) in TFA/TFAA=3:1 (0.4 mL) was added L-cysteine hydrochloride (9.52 mg, 0.060 mmol) at 25° C. After the addition was finished, the reaction was stirred at 50° C. for 0.5 h. The mixture was concentrated and purified by reverse phase HPLC (Column Boston Green ODS 150×30 mm×5 um, Condition water (0.1% TFA)-ACN Begin B 20 End B 50 Gradient Time (min) 10 100% B Hold Time (min) 2 Flow Rate (mL/min) 25 Injections 1) to give 2-((2S)-4-(7-(2-amino-5,7-difluoroquinolin-8-yl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-1-((E)-4-(4-methoxypiperidin-1-yl)but-2-enoyl)piperazin-2-yl)acetonitrile (Ex. 23). MS (ESI) [M+H]⁺ m/z: 776. ¹H NMR (500 MHz, ACETONITRILE-d3) δ 11.25 (br s, 2H), 8.32 (d, J=9.2 Hz, 1H), 7.24-7.02 (m, 2H), 6.94-6.64 (m, 2H), 5.65 (br dd, J=3.7, 11.0 Hz, 1H), 5.58-5.40 (m, 1H), 5.09-4.93 (m, 2H), 4.68-4.44 (m, 5H), 4.17 (br d, J=13.4 Hz, 2H), 4.02-3.78 (m, 4H), 3.73-3.47 (m, 4H), 3.40-3.23 (m, 7H), 3.15-3.03 (m, 2H), 2.93-2.79 (m, 2H), 2.66-2.44 (m, 2H), 2.34-1.97 (m, 7H), 1.70 (br d, J=11.6 Hz, 1H), 1.27 (br d, J=4.3 Hz, 1H).

To a stirred solution of 2-((2S)-4-(7-(2-(bis(2,4-dimethoxybenzyl)amino)-5,7-difluoroquinolin-8-yl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-1-((E)-4-(4-methoxypiperidin-1-yl)but-2-enoyl)piperazin-2-yl)acetonitrile (13 mg, 0.012 mmol) (Int-23h-P2) in TFA/TFAA=3:1 (0.4 mL) was added L-cysteine hydrochloride (9.52 mg, 0.060 mmol) at 25° C. After the addition was finished, the reaction was stirred at 50° C. for 0.5 h. The mixture was concentrated and purified by reverse phase HPLC (Column Boston Green ODS 150×30 mm×5 um Condition water (0.10% TFA)-ACN Begin B 20 End B 50 Gradient Time (min) 10 100% B Hold Time (min) 2 Flow Rate (mL/min) 25 Injections 1) to give 2-((2S)-4-(7-(2-amino-5,7-difluoroquinolin-8-yl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-1-((E)-4-(4-methoxypiperidin-1-yl)but-2-enoyl)piperazin-2-yl)acetonitrile (Ex. 23b). MS (ESI) [M+H]⁺ m/z: 776. ¹H NMR (500 MHz, ACETONITRILE-d3) δ 11.91-11.25 (m, 2H), 8.30 (d, J=9.2 Hz, 1H), 7.14 (t, J=10.1 Hz, 1H), 7.06 (d, J=9.8 Hz, 1H), 6.92-6.66 (m, 2H), 5.67 (br dd, J=4.0, 11.3 Hz, 1H), 5.57-5.39 (m, 1H), 5.02 (br d, J=13.4 Hz, 1H), 4.89 (br d, J=14.0 Hz, 1H), 4.77-4.41 (m, 4H), 3.94-3.70 (m, 7H), 3.66-3.42 (m, 4H), 3.41-3.20 (m, 7H), 3.14-2.81 (m, 4H), 2.68-2.43 (m, 2H), 2.37-1.95 (m, 7H), 1.79-1.61 (m, 1H), 1.28 (br s, 1H).

### Example 24: 2-((2S)-4-(2-(((R)-2,2-difluoro-1-(hydroxymethyl)cyclopropyl)methoxy)-7-(8-ethynylnaphthalen-1-yl)-7,8-dihydro-5H-pyrano[4,3-d]pyrimidin-4-yl)-1-((E)-4-(4-methoxypiperidin-1-yl)but-2-enoyl)piperazin-2-yl)acetonitrile (Ex. 24)

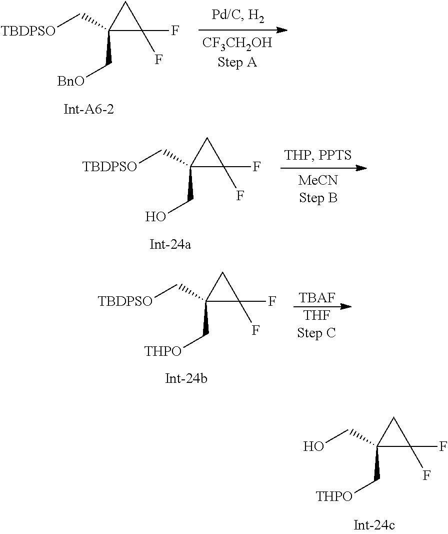

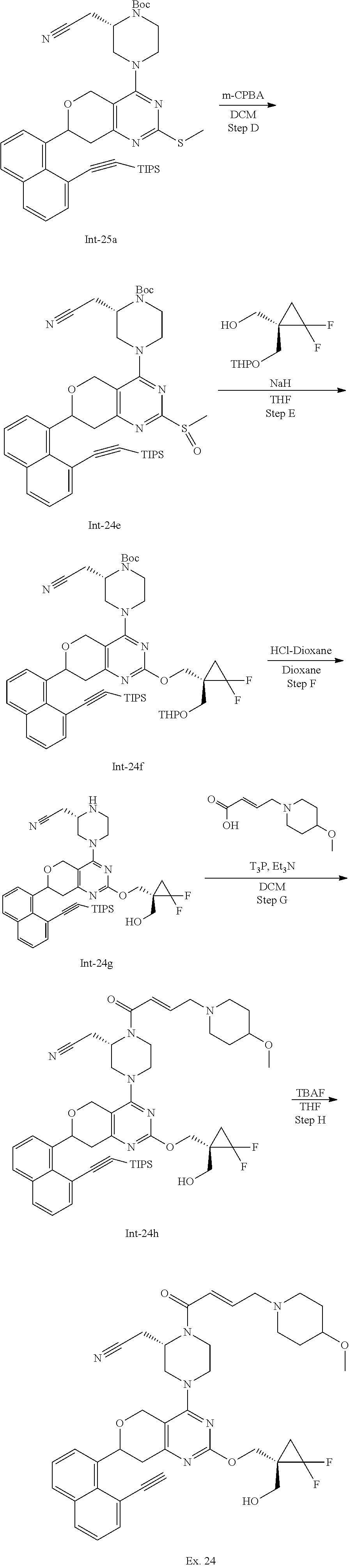

### Step A. (R)-(((tert-butyldiphenylsilyl)oxy)methyl)-2,2-difluorocyclopropyl)methanol (Int-24a)

To a solution of (R)-((1-((benzyloxy)methyl)-2,2-difluorocyclopropyl)methoxy)(tert-butyl)diphenylsilane (4 g, 8.57 mmol) in CF₃CH₂OH (120 mL) was added Pd/C (1 g, 0.940 mmol, 10% wt) at 25° C. under argon atmosphere. The mixture was stirred at 25° C. under H₂ atmosphere (40 psi) for 72 h. The mixture was filtered and the solvent was evaporated under reduced pressure to give the crude product. The crude product was purified by flash silica gel chromatography (ISCO®; 40 g SepaFlash® Silica Flash Column, Eluent of 5% ethyl acetate in petroleum ether gradient @ 25 mL/min) to give (R)-(1-(((tert-butyldiphenylsilyl)oxy)methyl)-2,2-difluorocyclopropyl)methanol (Int-24a). ¹H NMR (400 MHz, CDCl₃) δ 7.68 (d, J=6.8 Hz, 4H), 7.50-7.38 (m, 6H), 3.91-3.77 (m, 4H), 2.33 (t, J=6.0 Hz, 1H), 1.39-1.19 (m, 2H), 1.09 (s, 9H).

### Step B: tert-butyl(((1R)-2,2-difluoro-1-(((tetrahydro-2H-pyran-2-yl)oxy)methyl)cyclopropyl)methoxy)diphenylsilane (Int-24b)

To a stirred solution of (R)-(1-(((tert-butyldiphenylsilyl)oxy)methyl)-2,2-difluorocyclopropyl)methanol (Int-24a) (100 mg, 0.266 mmol) in MeCN (2 mL) was added 3,4-dihydro-2H-pyran (26.8 mg, 0.319 mmol) and PPTS (6.67 mg, 0.027 mmol) at 20° C., after the addition was finished, the reaction was stirred at 20° C. for 16 h. The crude product was purified by prep-TLC (Pet. ether/ethyl acetate 3:1 as eluent) to give tert-butyl(((1R)-2,2-difluoro-1-(((tetrahydro-2H-pyran-2-yl)oxy)methyl)cyclopropyl)methoxy)diphenylsilane (Int-24b). MS (ESI) [M+Na]⁺ m/z: 483.

### Step C: ((1S)-2,2-difluoro-1-(((tetrahydro-2H-pyran-2-yl)oxy)methyl)cyclopropyl)methanol (Int-24c)

To a stirred solution of tert-butyl(((1R)-2,2-difluoro-1-(((tetrahydro-2H-pyran-2-yl)oxy)methyl)cyclopropyl)methoxy)diphenylsilane (Int-24b) (500 mg, 1.085 mmol) in THF (5 mL) was added TBAF (1.085 mL, 1.085 mmol) at 20° C. After the addition was finished, the reaction was stirred at 20° C. for 16 h. The reaction was cooled to room temperature, then the mixture was extracted with EtOAc (20 mL×2). The combined organic layer was washed with brine, dried over Na₂SO₄, filtered and concentrated under reduced pressure to give crude product. The crude product was purified by flash silica gel chromatography (ISCO®; Agela® Flash Column Silica-CS (4 g) Eluent of 0˜30% Ethyl acetate/Petroleum ether gradient @ 40 mL/min) to give ((1S)-2,2-difluoro-1-(((tetrahydro-2H-pyran-2-yl)oxy)methyl)cyclopropyl)methanol (Int-24c). MS (ESI) [M+H]+ m/z: 223.

### Step D: tert-butyl (2S)-2-(cyanomethyl)-4-(2-(methylsulfinyl)-7-(8-((triisopropylsilyl)ethynyl)naphthalen-1-yl)-7,8-dihydro-5H-pyrano[4,3-d]pyrimidin-4-yl)piperazine-1-carboxylate (Int-24)

To a solution of tert-butyl (2S)-2-(cyanomethyl)-4-(2-(methylthio)-7-(8-((triisopropylsilyl)ethynyl)naphthalen-1-yl)-7,8-dihydro-5H-pyrano[4,3-d]pyrimidin-4-yl)piperazine-1-carboxylate (Int-25a) (50 mg, 0.070 mmol) in DCM (1 mL) was added m-CPBA (21.38 mg, 0.105 mmol) (85%) at 0° C., the reaction was stirred at 0° C. for 30 min. LCMS showed the reaction was finished. The reaction was diluted with EtOAc (10 mL), washed with NaHCO₃ (aq., 2 mL), the organic layer was dried over Na₂SO₄ and concentrated in vacuo. The residue was purified by Prep-TLC (SiO₂, Pet.ether/EtOAc=1:1) to give tert-butyl (2S)-2-(cyanomethyl)-4-(2-(methylsulfinyl)-7-(8-((triisopropylsilyl)ethynyl)naphthalen-1-yl)-7,8-dihydro-5H-pyrano[4,3-d]pyrimidin-4-yl)piperazine-1-carboxylate (Int-24e). MS (ESI) [M+H]+ m/z: 728.5.

### Step E: tert-butyl (2S)-2-(cyanomethyl)-4-(2-(((1S)-2,2-difluoro-1-(((tetrahydro-2H-pyran-2-yl)oxy)methyl)cyclopropyl)methoxy)-7-(8-((triisopropylsilyl)ethynyl)naphthalen-1-yl)-7,8-dihydro-5H-pyrano[4,3-d]pyrimidin-4-yl)piperazine-1-carboxylate (Int-24f)

To a solution of NaH (10.99 mg, 0.275 mmol)(60%) in THF (0.5 mL) was added ((1S)-2,2-difluoro-1-(((tetrahydro-2H-pyran-2-yl)oxy)methyl)cyclopropyl)methanol (Int-24c) (61.0 mg, 0.275 mmol). The mixture was stirred for 3 minutes at 20° C., and the resulting mixture was added to a solution of tert-butyl (2S)-2-(cyanomethyl)-4-(2-(methylsulfinyl)-7-(8-((triisopropylsilyl)ethynyl)naphthalen-1-yl)-7,8-dihydro-5H-pyrano[4,3-d]pyrimidin-4-yl)piperazine-1-carboxylate (Int-24e) (40 mg, 0.055 mmol) in THF (0.5 mL). The mixture was stirred at 20° C. for 30 minutes. The reaction was diluted with EtOAc (5 mL), washed with aqueous NH₄C¹ (1 mL), the organic layer was dried and concentrated in vacuo. The residue was purified by Prep-TLC (SiO₂, DCM/MeOH=10:1) to give tert-butyl (2S)-2-(cyanomethyl)-4-(2-(((1S)-2,2-difluoro-1-(((tetrahydro-2H-pyran-2-yl)oxy)methyl)cyclopropyl)methoxy)-7-(8-((triisopropylsilyl)ethynyl)naphthalen-1-yl)-7,8-dihydro-5H-pyrano[4,3-d]pyrimidin-4-yl)piperazine-1-carboxylate (Int-24f). MS (ESI) [M+H]+ m/z: 886.8.

### Step F: 2-((2S)-4-(2-(((R)-2,2-difluoro-1-(hydroxymethyl)cyclopropyl)methoxy)-7-(8-((triisopropylsilyl)ethynyl)naphthalen-1-yl)-7,8-dihydro-5H-pyrano[4,3-d]pyrimidin-4-yl)piperazin-2-yl)acetonitrile (Int-24g)

To a stirred solution of tert-butyl (2S)-2-(cyanomethyl)-4-(2-(((1S)-2,2-difluoro-1-(((tetrahydro-2H-pyran-2-yl)oxy)methyl)cyclopropyl)methoxy)-7-(8-((triisopropylsilyl)ethynyl)naphthalen-1-yl)-7,8-dihydro-5H-pyrano[4,3-d]pyrimidin-4-yl)piperazine-1-carboxylate (Int-24f) (30 mg, 0.035 mmol) in dioxane (1 mL) was added HCl in dioxane (0.1 mL, 0.035 mmol, 4N) at 20° C. After the addition was finished, the reaction was stirred at 20° C. for 16 h. The reaction was concentrated under reduced pressure to give the crude product 2-((2S)-4-(2-(((R)-2,2-difluoro-1-(hydroxymethyl)cyclopropyl)methoxy)-7-(8-((triisopropylsilyl)ethynyl)naphthalen-1-yl)-7,8-dihydro-5H-pyrano[4,3-d]pyrimidin-4-yl)piperazin-2-yl)acetonitrile (Int-24g). MS (ESI) [M+H]⁺ m/z: 702.5.

### Step G: 2-((2S)-4-(2-(((R)-2,2-difluoro-1-(hydroxymethyl)cyclopropyl)methoxy)-7-(8-((triisopropylsilyl)ethynyl)naphthalen-1-yl)-7,8-dihydro-5H-pyrano[4,3-d]pyrimidin-4-yl)-1-((E)-4-(4-methoxypiperidin-1-yl)but-2-enoyl)piperazin-2-yl)acetonitrile (Int-24h)

A mixture of 2-((2S)-4-(2-(((R)-2,2-difluoro-1-(hydroxymethyl)cyclopropyl)methoxy)-7-(8-((triisopropylsilyl)ethynyl)naphthalen-1-yl)-7,8-dihydro-5H-pyrano[4,3-d]pyrimidin-4-yl)piperazin-2-yl)acetonitrile (Int-24g) (21.14 mg, 0.030 mmol), (E)-4-(4-methoxypiperidin-1-yl)but-2-enoic acid (9 mg, 0.045 mmol), T3P (28.7 mg, 0.045 mmol) and TEA (0.021 mL, 0.151 mmol) in DCM (1.0 mL) was bubbled with N₂ for 1 minute. The reaction mixture was stirred at 25° C. for 16 h. The reaction mixture was concentrated in vacuo and the crude product was purified by preparative HPLC (Column: Phenomenex Synergi C18 150 mm×30 mm×4 μm; Condition: water (0.1% TFA)-MeCN Begin B 14, End B 44; Gradient Time (min): 11.5; 100% B Hold Time (min): 2; Flow Rate (mL/min) 25) to give 2-((2S)-4-(2-(((R)-2,2-difluoro-1-(hydroxymethyl)cyclopropyl)methoxy)-7-(8-((triisopropylsilyl)ethynyl)naphthalen-1-yl)-7,8-dihydro-5H-pyrano[4,3-d]pyrimidin-4-yl)-1-((E)-4-(4-methoxypiperidin-1-yl)but-2-enoyl)piperazin-2-yl)acetonitrile (Int-24h). MS (ESI) [M+H]⁺ m/z: 883.4

### Step H: 2-((2S)-4-(2-(((R)-2,2-difluoro-1-(hydroxymethyl)cyclopropyl)methoxy)-7-(8-ethynylnaphthalene-1-yl)-7,8-dihydro-5H-pyrano[4,3-d]pyrimidin-4-yl)-1-((E)-4-(4-methoxypiperidin-1-yl)but-2-enoyl)piperazin-2-yl)acetonitrile (Ex. 24)

To a stirred solution of 2-((2S)-4-(2-(((R)-2,2-difluoro-1-(hydroxymethyl)cyclopropyl)methoxy)-7-(8-((triisopropylsilyl)ethynyl)naphthalen-1-yl)-7,8-dihydro-5H-pyrano[4,3-d]pyrimidin-4-yl)-1-((E)-4-(4-methoxypiperidin-1-yl)but-2-enoyl)piperazin-2-yl)acetonitrile (Int-24h) (10 mg, 0.011 mmol) in THF (1 mL) was added TBAF (0.017 mL, 0.017 mmol) at 20° C. After the addition was finished, the reaction was stirred at 20° C. for 3 h. The mixture was concentrated in vacuo to give a residue. The crude product was purified by prep-HPLC (reverse preparative HPLC (Column: Waters XSELECT C18 150×30 mm×5 um; Condition: water (0.1% TFA)-MeCN; Begin B-End B: 21-41; Gradient Time (min): 10; 100% B Hold Time (min): 1; Flow Rate (mL/min): 25)) to give the title compound 2-((2S)-4-(2-(((R)-2,2-difluoro-1-(hydroxymethyl)cyclopropyl)methoxy)-7-(8-ethynylnaphthalen-1-yl)-7,8-dihydro-5H-pyrano[4,3-d]pyrimidin-4-yl)-1-((E)-4-(4-methoxypiperidin-1-yl)but-2-enoyl)piperazin-2-yl)acetonitrile (Ex. 24). MS (ESI) [M+H]+m/z: 727.3 ¹H NMR (400 MHz, CDCl₃) δ 7.77-7.95 (m, 3H), 7.46-7.58 (m, 1H), 7.40 (br d, J=7.82 Hz, 1H), 7.23 (br s, 1H), 6.74-7.00 (m, 2H), 6.47 (br, 1H), 4.96 (br s, 2H), 4.64-4.85 (m, 2H), 4.33-4.59 (m, 2H), 3.96 (br s, 1H), 3.51-3.83 (m, 4H), 3.05-3.46 (m, 7H), 2.65-2.98 (m, 4H), 2.23 (br s, 2H), 1.82-2.05 (m, 2H), 1.63 (br s, 2H), 1.35 (br s, 6H).

### Example 25: 2-((S)-4-((S)-7-(8-ethynylnaphthalen-1-yl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-1-((E)-4-(4-methoxypiperidin-1-yl)but-2-enoyl)piperazin-2-yl)acetonitrile (Ex. 25a/b)

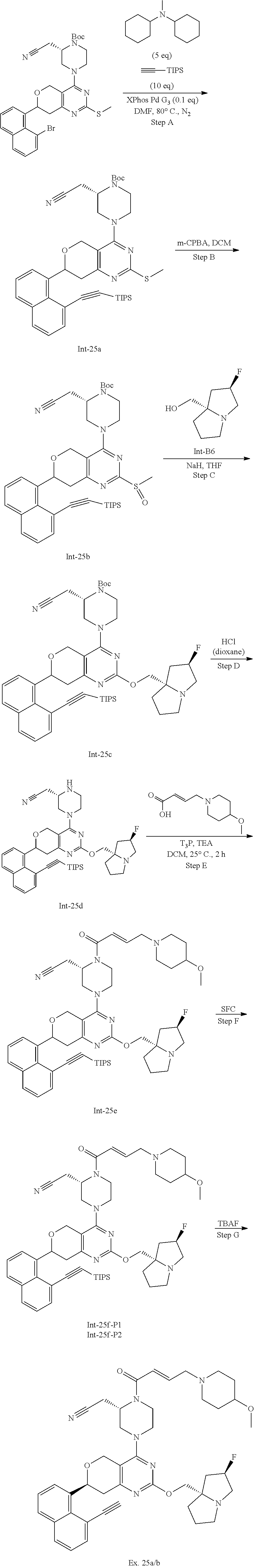

### Step A: tert-butyl (2S)-2-(cyanomethyl)-4-(2-(methylthio)-7-(8-((triisopropylsilyl)ethynyl)naphthalen-1-yl)-7,8-dihydro-5H-pyrano[4,3-d]pyrimidin-4-yl)piperazine-1-carboxylate (Int-25a)

To a solution of tert-butyl (2S)-4-(7-(8-bromonaphthalen-1-yl)-2-(methylthio)-7,8-dihydro-5H-pyrano[4,3-d]pyrimidin-4-yl)-2-(cyanomethyl)piperazine-1-carboxylate (430 mg, 0.704 mmol) in DMF (5 mL) were added ethynyltriisopropylsilane (1284 mg, 7.04 mmol), N-cyclohexyl-N-methylcyclohexanamine (413 mg, 2.113 mmol) and XPhos Pd G3 (59.6 mg, 0.070 mmol) at 20° C. The mixture was stirred at 80° C. for 10 h under nitrogen atmosphere. The reaction was diluted with EtOAc (50 mL) and washed with brine (5 mL×3). The organic layer was dried over Na₂SO₄ and concentrated in vacuo. The residue was purified by flash silica gel chromatography (ISCO®; Agela® Flash Column Silica-CS (12 g), Eluent of 0˜10% ethyl acetate/petroleum ether gradient @ 30 mL/min) to give tert-butyl (2S)-2-(cyanomethyl)-4-(2-(methylthio)-7-(8-((triisopropylsilyl)ethynyl)naphthalen-1-yl)-7,8-dihydro-5H-pyrano[4,3-d]pyrimidin-4-yl)piperazine-1-carboxylate (Int-25a). MS (ESI) [M+H]+ m/z 712.

### Step B: tert-butyl (2S)-2-(cyanomethyl)-4-(2-(methylsulfinyl)-7-(8-((triisopropylsilyl)ethynyl)naphthalen-1-yl)-7,8-dihydro-5H-pyrano[4,3-d]pyrimidin-4-yl)piperazine-1-carboxylate (Int-25b)

To a solution of tert-butyl (2S)-2-(cyanomethyl)-4-(2-(methylthio)-7-(8-((triisopropylsilyl)ethynyl)naphthalen-1-yl)-7,8-dihydro-5H-pyrano[4,3-d]pyrimidin-4-yl)piperazine-1-carboxylate (Int-25a) (100 mg, 0.140 mmol) in DCM (2 mL) was added m-CPBA (45.4 mg, 0.211 mmol) (85%) at 0° C., the reaction was stirred at 0° C. for 30 min. The reaction was diluted with EtOAc (10 mL), washed with NaHCO₃ (aq., 2 mL), the organic layer was dried over Na₂SO₄ and concentrated in vacuo. The residue was purified by Prep-TLC (SiO₂, Pet. ether/EtOAc=1:1) to give tert-butyl (2S)-2-(cyanomethyl)-4-(2-(methylsulfinyl)-7-(8-((triisopropylsilyl)ethynyl)naphthalen-1-yl)-7,8-dihydro-5H-pyrano[4,3-d]pyrimidin-4-yl)piperazine-1-carboxylate (a mixture of sulphone and sulfoxide) (Int-25b). MS (ESI) [M+H]+ m/z 728.

### Step C: tert-butyl (2S)-2-(cyanomethyl)-4-(2-(((2R,7aS)-2-fluorotetrahydro-1H-pyrrolizin-7a(5H)-yl)methoxy)-7-(8-((triisopropylsilyl)ethynyl)naphthalen-1-yl)-7,8-dihydro-5H-pyrano[4,3-d]pyrimidin-4-yl)piperazine-1-carboxylate (Int-25c)

To a solution of ((2R,7aS)-2-fluorotetrahydro-1H-pyrrolizin-7a(5H)-yl)methanol (Int-B6, 82 mg, 0.515 mmol) in THF (0.5 mL) was added NaH (20.60 mg, 0.515 mmol) (60% in mineral oil), and the mixture was stirred at 20° C. for 2 min. Then the mixture was added to a solution of tert-butyl (2S)-2-(cyanomethyl)-4-(2-(methylsulfinyl)-7-(8-((triisopropylsilyl)ethynyl)naphthalen-1-yl)-7,8-dihydro-5H-pyrano[4,3-d]pyrimidin-4-yl)piperazine-1-carboxylate (Int-25b) (75 mg, 0.103 mmol) in THF (1 mL). The reaction was stirred at 20° C. for 5 min. The reaction was diluted with EtOAc (10 mL), quenched with NH₄C¹ (aq., 1 mL), the organic layer was dried and concentrated, the residue was purified by preparative TLC (SiO₂, DCM: MeOH=10:1) to give tert-butyl (2S-2-(cyanomethyl)-4-(2-(((2S,7aR)-2-fluorotetrahydro-1H-pyrrolizin-7a(5H)-yl)methoxy)-7-(8-((triisopropylsilyl)ethynyl)naphthalen-1-yl)-7,8-dihydro-5H-pyrano[4,3-d]pyrimidin-4-yl)piperazine-1-carboxylate (Int-25c). MS (ESI) [M+H]⁺: m/z 823.

### Step D: 2-((2S)-4-(2-(((2R,7aS)-2-fluorotetrahydro-1H-pyrrolizin-7a(5H)-yl)methoxy)-7-(8-((triisopropylsilyl)ethynyl)naphthalen-1-yl)-7,8-dihydro-5H-pyrano[4,3-d]pyrimidin-4-yl)piperazin-2-yl)acetonitrile (Int-25d)

A solution of tert-butyl (2S)-2-(cyanomethyl)-4-(2-(((2R,7aS)-2-fluorotetrahydro-1H-pyrrolizin-7a(5H)-yl)methoxy)-7-(8-((triisopropylsilyl)ethynyl)naphthalen-1-yl)-7,8-dihydro-5H-pyrano[4,3-d]pyrimidin-4-yl)piperazine-1-carboxylate (Int-25c27c) (40 mg, 0.049 mmol) in HCl/Dioxane (4 M) (1.0 mL) was stirred at 20° C. for 1 h. LC-MS showed all the starting material was consumed completely. The reaction mixture was concentrated under reduced pressure to give 2-((2S)-4-(2-(((2R,7aS)-2-fluorotetrahydro-1H-pyrrolizin-7a(5H)-yl)methoxy)-7-(8-((triisopropylsilyl)ethynyl)naphthalen-1-yl)-7,8-dihydro-5H-pyrano[4,3-d]pyrimidin-4-yl)piperazin-2-yl)acetonitrile (Int-25d), which was used without further purification. MS (ESI) [M+H]+: m/z 723.

### Step E: 2-((2S)-4-(2-(((2R,7aS)-2-fluorotetrahydro-1H-pyrrolizin-7a(5H)-yl)methoxy)-7-(8-((triisopropylsilyl)ethynyl)naphthalen-1-yl)-7,8-dihydro-5H-pyrano[4,3-d]pyrimidin-4-yl)-1-((E)-4-(4-methoxypiperidin-1-yl)but-2-enoyl)piperazin-2-yl)acetonitrile (Int-25e)

A solution of triethylamine (1.680 mL, 12.12 mmol), (E)-4-bromobut-2-enoic acid (1 g, 6.06 mmol) and 4-methoxypiperidine (0.698 g, 6.06 mmol) in THF (10 mL) was stirred at 20° C. for 3 h. The reaction was diluted with EtOAc (20 mL), filtered, and the filtrate was concentrated in vacuo to afford (E)-4-(4-methoxypiperidin-1-yl)but-2-enoic acid, which was used directly in the next step. MS (ESI) [M+H]+: m/z 199.

To a solution of 2-((2S)-4-(2-(((2R,7aS)-2-fluorotetrahydro-1H-pyrrolizin-7a(5H)-yl)methoxy)-7-(8-((triisopropylsilyl)ethynyl)naphthalen-1-yl)-7,8-dihydro-5H-pyrano[4,3-d]pyrimidin-4-yl)piperazin-2-yl)acetonitrile (Int-25d) (88 mg, 0.122 mmol), (E)-4-(4-methoxypiperidin-1-yl)but-2-enoic acid (48.5 mg, 0.243 mmol) and TEA (0.085 mL, 0.609 mmol) in DCM (3 mL) was added T3P (155 mg, 0.243 mmol) (50% in EtOAc). The reaction mixture was stirred at 20° C. for 2 h. The reaction mixture was diluted with DCM (3 mL), washed with brine (1 mL), the organic layer was concentrated under reduced pressure to give a residue. The residue was purified by Prep-TLC (SiO₂, DCM/MeOH=10:1, v/v) to give 2-((2S)-4-(2-(((2R,7aS)-2-fluorotetrahydro-1H-pyrrolizin-7a(5H)-yl)methoxy)-7-(8-((triisopropylsilyl)ethynyl)naphthalen-1-yl)-7,8-dihydro-5H-pyrano[4,3-d]pyrimidin-4-yl)-1-((E)-4-(4-methoxypiperidin-1-yl)but-2-enoyl)piperazin-2-yl)acetonitrile (Int-25e). MS (ESI) [M+H]⁺: m/z 904.

### Step F: 2-((2S)-4-(2-(((2R,7aS)-2-fluorotetrahydro-1H-pyrrolizin-7a(5H)-yl)methoxy)-7-(8-((triisopropylsilyl)ethynyl)naphthalen-1-yl)-7,8-dihydro-5H-pyrano[4,3-d]pyrimidin-4-yl)-1-((E)-4-(4-methoxypiperidin-1-yl)but-2-enoyl)piperazin-2-yl)acetonitrile (Int-25f-P1 and Int-25f-P2)

2-((2S)-4-(2-(((2R,7aS)-2-fluorotetrahydro-1H-pyrrolizin-7a(5H)-yl)methoxy)-7-(8-((triisopropylsilyl)ethynyl)naphthalen-1-yl)-7,8-dihydro-5H-pyrano[4,3-d]pyrimidin-4-yl)-1-((E)-4-(4-methoxypiperidin-1-yl)but-2-enoyl)piperazin-2-yl)acetonitrile (Int-25e) (80 mg, 0.088 mmol) was separated by preparative SFC (Method Column B: condition: 0.1% NH₃H₂O IPA; Begin B 45%; Begin B 45% Gradient Time (min) 100% B Hold Time (min) Flow Rate (mL/min) 80 mL/min) to give 2-((2S)-4-(2-(((2R,7aS)-2-fluorotetrahydro-1H-pyrrolizin-7a(5H)-yl)methoxy)-7-(8-((triisopropylsilyl)ethynyl)naphthalen-1-yl)-7,8-dihydro-5H-pyrano[4,3-d]pyrimidin-4-yl)-1-((E)-4-(4-methoxypiperidin-1-yl)but-2-enoyl)piperazin-2-yl)acetonitrile (Int-25f-P1, the first eluting isomer from SFC) and 2-((2S)-4-(2-(((2R,7aS)-2-fluorotetrahydro-1H-pyrrolizin-7a(5H)-yl)methoxy)-7-(8-((triisopropylsilyl)ethynyl)naphthalen-1-yl)-7,8-dihydro-5H-pyrano[4,3-d]pyrimidin-4-yl)-1-((E)-4-(4-methoxypiperidin-1-yl)but-2-enoyl)piperazin-2-yl)acetonitrile (Int-25f-P2, the second eluting isomer from SFC). MS (ESI) [M+H]⁺: m/z 904.5.

### Step G: 2-((S)-4-((S)-7-(8-ethynylnaphthalen-1-yl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-1-((E)-4-(4-methoxypiperidin-1-yl)but-2-enoyl)piperazin-2-yl)acetonitrile (Ex. 25a/b)

To a solution of 2-((2S)-4-(2-(((2R,7aS)-2-fluorotetrahydro-1H-pyrrolizin-7a(5H)-yl)methoxy)-7-(8-((triisopropylsilyl)ethynyl)naphthalen-1-yl)-7,8-dihydro-5H-pyrano[4,3-d]pyrimidin-4-yl)-1-((E)-4-(4-methoxypiperidin-1-yl)but-2-enoyl)piperazin-2-yl)acetonitrile (30 mg, 0.033 mmol) (Int-25f-P1) in THF (0.2 mL) was added TBAF (0.066 mL, 0.066 mmol) (1 M in THF). The reaction mixture was stirred at 25° C. for 1 h. The reaction mixture was purified by Prep-TLC (SiO₂, DCM/7 M NH₃/MeOH=20:1, v/v) to give the crude product, which was re-purified by Prep-HPLC (Column Welch Xtimate BEH C18 100×25 mm×5 um, Condition water (0.2% FA)-ACN Begin B 7, End B 27 Gradient Time (min) 12, 100% B Hold Time (min) 2 Flow Rate (mL/min) 25, Injections 1) to give 2-((2S)-4-((S)-(7-(8-ethynylnaphthalen-1-yl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-1-((E)-4-(4-methoxypiperidin-1-yl)but-2-enoyl)piperazin-2-yl)acetonitrile (Ex. 25a) as a single atropisomer. ¹H NMR (400 MHz, MeOD) δ 8.00 (d, J=8.2 Hz, 1H), 7.92 (t, J=7.7 Hz, 2H), 7.85 (dd, J=1.2, 7.2 Hz, 1H), 7.58 (t, J=7.7 Hz, 1H), 7.49 (t, J=7.7 Hz, 1H), 6.93-6.66 (m, 3H), 5.50-5.29 (m, 1H), 5.19-4.94 (m, 2H), 4.59 (br s, 2H), 4.52-4.38 (m, 1H), 4.33 (br s, 2H), 4.16-3.87 (m, 2H), 3.85 (s, 1H), 3.65-3.39 (m, 5H), 3.36 (s, 3H), 3.32-3.26 (m, 4H), 3.24-3.04 (m, 3H), 2.85 (br s, 2H), 2.75 (br dd, J=10.9, 18.1 Hz, 1H), 2.55-2.17 (m, 5H), 2.17-2.06 (m, 2H), 1.97 (br d, J=12.4 Hz, 3H), 1.67 (br d, J=7.6 Hz, 2H). MS (ESI) [M+H]+: m/z 748.

Ex. 25b was prepared as in Ex. 25a using Int-25f-P2 above. ¹H NMR (400 MHz, CD₃OD) δ 8.00 (d, J=8.2 Hz, 1H), 7.93 (dd, J=7.8, 11.7 Hz, 2H), 7.88-7.82 (m, 1H), 7.58 (t, J=7.7 Hz, 1H), 7.48 (t, J=7.7 Hz, 1H), 6.96-6.62 (m, 3H), 5.43-5.23 (m, 1H), 5.20-4.89 (m, 3H), 4.84-4.45 (m, 2H), 4.27-3.96 (m, 5H), 3.87 (s, 1H), 3.83-3.43 (m, 3H), 3.35 (s, 3H), 3.26 (br s, 3H), 3.11-3.01 (m, 2H), 2.94-2.68 (m, 5H), 2.40-2.12 (m, 5H), 2.08-1.87 (m, 6H), 1.64 (br d, J=8.9 Hz, 2H). MS (ESI) [M+H]⁺: m/z 748.

### Example 26: 2-((2S)-4-(2′-(((2R,7aS)-2-fluorotetrahydro-1H-pyrrolizin-7a(5H)-yl)methoxy)-3,4,5′,6′-tetrahydro-2H-spiro[naphthalene-1,7′-pyrano[2,3-d]pyrimidin]-4′-yl)-1-((E)-4-(4-methoxypiperidin-1-yl)but-2-enoyl)piperazin-2-yl)acetonitrile (Ex. 27)

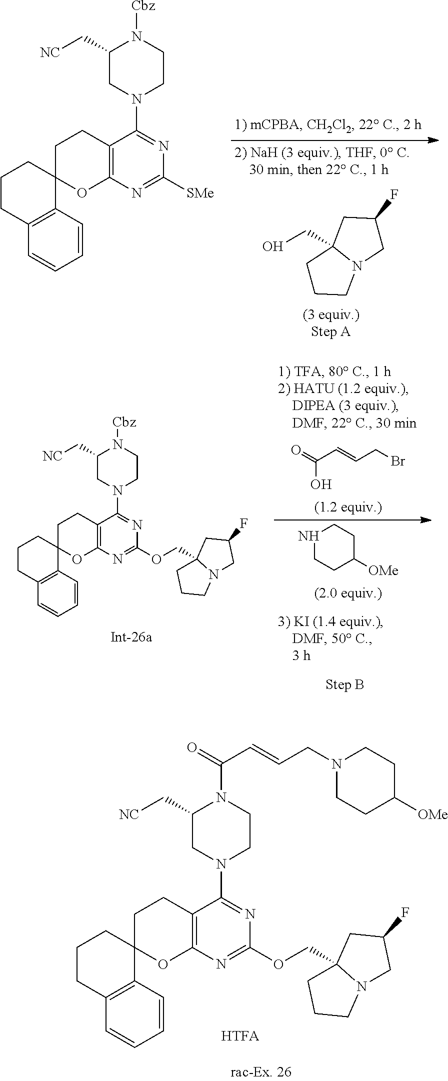

### Step A: benzyl (2S)-2-(cyanomethyl)-4-(2′-(((2R,7aS)-2-fluorotetrahydro-1H-pyrrolizin-7a(5H)-yl)methoxy)-3,4,5′,6′-tetrahydro-2H-spiro[naphthalene-1,7′-pyrano[2,3-d]pyrimidin]-4′-yl)piperazine-1-carboxylate (Int-26a)

A mixture of benzyl (2S)-2-(cyanomethyl)-4-(2′-(methylthio)-3,4,5′,6′-tetrahydro-2H-spiro[naphthalene-1,7′-pyrano[2,3-d]pyrimidin]-4′-yl)piperazine-1-carboxylate (200 mg, 0.361 mmol) and 3-chlorobenzoperoxoic acid (249 mg, 1.082 mmol) in DCM (1800 μl) was stirred at room temperature for 3 hours. The reaction mixture was diluted with DCM, filtered and the filtrate was washed with saturated, aqueous NaHCO₃ solution. The combined organic layers were concentrated to yield benzyl (2R)-2-(cyanomethyl)-4-(2′-(methylsulfonyl)-3,4,5′,6′-tetrahydro-2H-spiro[naphthalene-1,7′-pyrano[2,3-d]pyrimidin]-4′-yl)piperidine-1-carboxylate. The crude mixture was used in the next step without further purification. MS (ESI): [M+H]⁺ m/z: 588.

A vial was loaded with NaH (43.0 mg, 1.083 mmol), suspended in 0.2 mL of THF and cooled down to 0° C. with an ice bath. A solution of ((2R,7aS)-2-fluorotetrahydro-1H-pyrrolizin-7a(5H)-yl)methanol (172.0 mg μL, 1.083 mmol) in 1.2 mL of THF was added dropwise. The mixture was stirred for 5 minutes. Then, a solution of benzyl (2R)-2-(cyanomethyl)-4-(2′-(methylsulfonyl)-3,4,5′,6′-tetrahydro-2H-spiro[naphthalene-1,7′-pyrano[2,3-d]pyrimidin]-4′-yl)piperidine-1-carboxylate (0.361 mmol, dissolved in 600 μL) was added dropwise. When the addition was finished, the ice bath was removed, and the mixture was stirred at room temperature for 20 minutes. The reaction was diluted with MeOH, and concentrated. The crude material was purified by 40 g silica gel column with a gradient of 0-20% of methanol in DCM to yield the title compound (Int-26a). MS (ESI): [M+H]⁺ m/z: 665.

### Step B: 2-((2S)-4-(2′-(((2R,7aS)-2-fluorotetrahydro-1H-pyrrolizin-7a(5H)-yl)methoxy)-3,4,5′,6′-tetrahydro-2H-spiro[naphthalene-1,7′-pyrano[2,3-d]pyrimidin]-4′-yl)-1-((E)-4-(4-methoxypiperidin-1-yl)but-2-enoyl)piperazin-2-yl)acetonitrile (Ex. 26)

A vial was loaded with benzyl (2S)-2-(cyanomethyl)-4-(2′-(((2R,7aS)-2-fluorotetrahydro-1H-pyrrolizin-7a(5H)-yl)methoxy)-3,4,5′,6′-tetrahydro-2H-spiro[naphthalene-1,7′-pyrano[2,3-d]pyrimidin]-4′-yl)piperazine-1-carboxylate (Int-26a) (43 mg, 0.064 mmol) and TFA (650 μL). The vial was heated to 80° C. for 1 h. The reaction was quenched with a saturated water solution Na₂CO₃ until the pH turned basic. The mixture was extracted with EtOAc (3×30 mL). The combined organic layers were dried over Na₂SO₄ and concentrated under reduced pressure, affording 2-((2S)-4-(2′-(((2R,7aS)-2-fluorotetrahydro-1H-pyrrolizin-7a(5H)-yl)methoxy)-3,4,5′,6′-tetrahydro-2H-spiro[naphthalene-1,7′-pyrano[2,3-d]pyrimidin]-4′-yl)piperazin-2-yl)acetonitrile. The crude material was taken to the next step without further purification (34.5 mg, >98% yield). MS (ESI): [M+H]⁺ m/z: 532.

A vial was loaded with 2-((2S)-4-(2′-(((2R,7aS)-2-fluorotetrahydro-1H-pyrrolizin-7a(5H)-yl)methoxy)-3,4,5′,6′-tetrahydro-2H-spiro[naphthalene-1,7′-pyrano[2,3-d]pyrimidin]-4′-yl)piperazin-2-yl)acetonitrile (34.5 mg, 0.064 mmol), (E)-4-bromobut-2-enoic acid (21 mg, 0.127 mmol) and DIPEA (44.6 μL, 0.256 mmol). The solids were dissolved in 500 μL of DMF at room temperature. A solution of HATU (48.7 mg, 0.128) in 500 μL of DMF was added to the mixture and the reaction was stirred for 1.5 h. After this time, the reaction was diluted with water and extracted with EtOAc. The solvent was removed under vacuum, affording 2-((2S)-1-((E)-4-bromobut-2-enoyl)-4-(2′-(((2R,7aS)-2-fluorotetrahydro-1H-pyrrolizin-7a(5H)-yl)methoxy)-3,4,5′,6′-tetrahydro-2H-spiro[naphthalene-1,7′-pyrano[2,3-d]pyrimidin]-4′-yl)piperazin-2-yl)acetonitrile. The crude material was taken to the next step without further purification. MS (ESI): [M+H]⁺ m/z: 680.

A vial was loaded with 2-((2S)-1-((E)-4-bromobut-2-enoyl)-4-(2′-(((2R,7aS)-2-fluorotetrahydro-1H-pyrrolizin-7a(5H)-yl)methoxy)-3,4,5′,6′-tetrahydro-2H-spiro[naphthalene-1,7′-pyrano[2,3-d]pyrimidin]-4′-yl)piperazin-2-yl)acetonitrile (4.8 mg, 7.56 μmol), 4-methoxypiperidine (15.0 mg, 0.130 mmol) and KI (14.9 mg, 0.090 mmol). The contents were dissolved in 640 μL of DMF. The reaction mixture was heated to 50° C. for 3 h. The reaction mixture was diluted with DMF up to a final volume of 2 mL, filtered, and purified via reverse phase HPLC (elution: acetonitrile/water, 0.1% formic acid) to give the title compound (Ex. 26). ¹H NMR (499 MHz, CD₃OD) δ 7.45-7.37 (m, 1H), 7.29-7.20 (m, 2H), 7.18 (d, J=7.2 Hz, 1H), 7.13-6.93 (m, 1H), 6.84-6.72 (m, 1H), 5.65-5.45 (m, 1H), δ 5.17-4.70 (m, 2H), 4.66-4.44 (m, 4H), 4.33-3.71 (m, 6H), 3.71-3.41 (m, 5H), 3.38 (s, 3H), 3.31-3.18 (m, 3H), 3.18-2.78 (m, 6H), 2.75-2.50 (m, 3H), 2.48-2.26 (m, 3H), 2.26-2.08 (m, 6H), 2.08-1.80 (m, 3H), 1.80-1.51 (m, 1H). MS (ESI): [M+H]⁺ m/z: 714 (M+H)⁺.

### Biochemical and Cellular Data

### Biological Assays

Compounds 1-21 were tested using the assay descriptions in Test Examples 1-3.

Test Example 1: Evaluation of inhibitory activity of compounds on KRAS G12C nucleotide (GDP-GTP) exchange reaction in vitro

Recombinant KRAS G12C mutant protein (amino acids 1-169, SEQ ID NO: 1) and cleaved recombinant SOS1 (amino acids 564-1049, SEQ ID NO:2) proteins were expressed in E. coli and purified by affinity chromatography.

To prepare BODIPY FL GDP-bound KRAS G12C protein, 50 μM KRAS G12C protein was incubated with 0.5 mM BODIPY FL GDP in a loading buffer (20 mM Tris-HCl (pH 7.5), 50 mM NaCl, 1 mM DTT and 2.5 mM EDTA) for 1 hour on ice. After the incubation, MgCl₂ was added to a final concentration of 10 mM, followed by incubation at room temperature for 30 minutes. The mixture was allowed to pass through a NAP-5 column to remove free nucleotides and purified BODIPY FL GDP-bound KRAS G12C protein was used for compound evaluation.

For the measurement of the inhibitory activity of compounds on GDP-GTP exchange rate of recombinant KRAS G12C, BODIPY FL GDP-bound KRAS G12C protein was incubated with various concentrations of compound in a reaction buffer (20 mM Tris-HCl (pH 7.5), 100 mM NaCl, 1 mM MgCl₂, 2 mM DTT, 0.1% Tween 20) at 25° C. for 1 hour. After the incubation, recombinant SOS1 and Guanosine-5′-[(β,γ)-imido]triphosphate, Tetralithium salt (GMPPNP) (Jena Bioscience GmbH) were added and incubated at room temperature for 30 minutes to proceed SOS1-dependent GDP-GTP exchange reaction on KRAS G12C. Replacement of BODIPY FL GDP by GMPPNP was measured by calculating the ratio of fluorescence intensities of BODIPY FL before and after the exchange reaction. Inhibition % was calculated by setting the fluorescence ratio from the reaction without test compound (DMSO control) and the fluorescence ratio from the reaction without SOS1 and GMPPNP as 0% and 100% inhibition, respectively. IC50 values were calculated from dose titration curve using curve fitting by XLfit software (IDBS). Table A shows the results.

### Test Example 2: Evaluation of Inhibitory Activity of Compounds on Interaction Between KRAS G12C and cRAF (In Vitro)

Interaction between recombinant KRAS G12C mutant protein (SEQ ID NO: 1) and cRAF RAS-binding domain (cRAF-RBD, amino acids 50-132, Jena Biosciences GmbH) was measured using Alpha technology (PerkinElmer Inc.).

Recombinant KRAS G12C protein with an N-terminal His-tag was expressed in E. coli and purified by affinity chromatography. To prepare GTP-bound form and GDP-bound form of KRAS G12C protein, 50 μM KRAS G12C protein was incubated with 1 mM GMPPNP (Jena Bioscience GmbH) and 1 mM GDP, respectively, in a loading buffer (20 mM Tris-HCl (pH 7.5), 50 mM NaCl, 1 mM DTT and 2.5 mM EDTA) for 1 hour on ice. After the incubation, MgCl₂ was added to a final concentration of 10 mM, followed by incubation at room temperature for 30 minutes. The mixture was allowed to pass through a NAP-5 column to remove free nucleotides and purified, and the resultant nucleotide-bound KRAS G12C protein was used for compound evaluation.

For the measurement of the inhibitory activity of compounds on interaction between KRAS G12C and cRAF-RBD, GMPPNP-bound KRAS G12C protein was incubated with various concentrations of compound in a reaction buffer (20 mM Tris-HCl (pH 7.5), 100 mM NaCl, 1 mM MgCl₂, 1 mM TCEP, 0.1% Tween 20) at 25° C. for 1 hour. After the incubation, recombinant cRAF-RBD and Alpha detection reagents were added and incubated at room temperature for 1.5 hours for binding. Interaction of KRAS G12C and cRAF-RBD was monitored by measuring Alpha signal. Inhibition % was calculated using the signal from the reaction without test compound (DMSO control) as 0% inhibition and the signal from the reaction using GDP-bound KRAS G12C in place of GMPPNP-bound KRAS G12C as 100% inhibition. IC50 values were calculated from dose titration curve using curve fitting by XLfit software (IDBS). Table A shows the results.

### Test Example 3: Assay of Growth Inhibition Activity on KRAS-G12C Mutant Cell Line (MIA PaCa-2) (In Vitro)

MIA PaCa-2 cells (provided by Sumitomo Dainippon Pharma Co., Ltd.), which are a KRAS-G12C mutant human pancreas cancer cell line, were suspended in a 10% fetal bovine serum-containing RPMI1640 medium (manufactured by Fujifilm Wako Pure Chemical Corporation.). The cell suspension was seeded into each well of a 384-well U bottom microplate and cultured in an incubator containing 5% CO₂ gas at 37° C. for 1 day. The compounds obtained in the Examples section were used as test compounds and were dissolved in DMSO, respectively, and each test compound was diluted with DMSO to give a concentration 500 times the final concentration. The resultant solution of the test compound in DMSO was diluted with the medium used for suspending cells and added to each well of the cell-culture plate to give a DMSO final concentration of 0.2%, followed by culture in an incubator containing 5% CO₂ gas at 37° C. for another 3 days. The cell count after 3-day culture in the presence of the test compound was measured using CellTiter-Glo 3D Reagent (manufactured by Promega Corporation). To all cells were added with CellTiter-Glo 3D Reagent and mixed for 10 minutes. 30 minutes after mixing, luminescence was measured by a plate reader. The growth inhibition rate was calculated from the following equation, and the concentration of the test compound at which 50% inhibition was achieved (IC50 (nM)) was determined. Table A shows the results.

$$Growth Inhibition Rate ( \% ) = ( C - T ) / ( C ) \times 100$$

T: the emission intensity in a well into which a test compound was added.
C: the emission intensity in a well into which a test compound was not added.

TABLE A
Biochemical and Cellular Data

| | Test Example 1 | Test Example 2 | Test Example 3 |
|---|---|---|---|
| Example | GNE IC₅₀ | cRaf IC₅₀ | CTX MiaPaCa IC₅₀ |
| 1 | 1.02 | 25.42 | 7.15 |
| 2 | 3.53 | 43672 | 111.03 |
| 3 | 5.22 | 11470 | 100.17 |
| 4 | 7.19 | 9575 | 333.29 |
| 5 | 2.63 | 2819.94 | 176.72 |
| 6 | 15.36 | 1255.99 | 268.34 |
| 7 | 0.88 | 50.59 | 4.36 |
| 8 | 0.54 | 64.4 | 6.4 |
| 9 | 1.27 | 133.33 | 40.44 |
| 10 | 0.73 | 106.59 | 10.14 |
| 11 | 0.81 | 181.63 | 21.74 |
| 12 | 6.7 | 308.8 | 170.11 |
| 13 | 0.91 | 51.31 | 6.21 |
| 14 | 0.82 | 530.82 | 7.88 |
| 15 | 103.31 | >6667 | 1922.42 |
| 16 | 337.54 | >6667 | >10000 |
| 17 | 255.09 | >6667 | 542.54 |
| 18 | 58.67 | >6667 | 4463.97 |
| 19 | 46.69 | 3550.15 | 8251.91 |
| 20 | 174.3 | >6667 | 3350.08 |
| 21 | 287.16 | >6667 | 1125.63 |

Compounds 22-26 were tested using the assay descriptions in Test Examples 4-5.

### Test Example 4: Procedure for SOS-Catalyzed Nucleotide Exchange Assay

The SOS-catalyzed nucleotide exchange assay utilizes a preformed complex of recombinant biotinylated KRAS protein containing G12C/C51S/C80L/C118S mutations (SEQ ID NO: 3) (referred to as Biotinylated KRAS G12C protein hereafter), Bodipy-GDP, and Terbium-streptavidin. Compounds are added to this complex and then after a 60-minute incubation time the mixture is treated with SOS and unlabeled GTP. Small molecule inhibitors stabilize the Bodipy-GDP complex whereas the untreated protein rapidly exchanges Bodipy-GDP for unlabeled GTP resulting in reduced TR-FRET signal.

Biotinylated KRAS G12C protein is diluted to 2 μM in an EDTA buffer (20 mM HEPES, 50 mM sodium chloride, 10 mM EDTA, and 0.01% Tween) and incubated at room temperature for one hour. This mixture is then further diluted to 90 nM in an assay buffer (20 mM HEPES, 150 mM sodium chloride, 10 mM magnesium chloride, and 0.005% Tween) containing 15 nM of Terbium-Streptavidin (Invitrogen, catalog #PV3577) and 900 nM of Bodipy-GDP (Invitrogen, G22360) and incubated at room temperature for six hours. This solution is referred to as Biotinylated KRAS G12C stock solution and is then diluted to 9 nM in assay buffer to give the KRAS G12C assay solution.

Each test compound (10 mM stock in DMSO) is diluted in DMSO to make a 10-point, 3-fold dilution series in a 384-well low dead volume microplate (Labcyte, catalog #LP-0200). Once titrations are made, 10 nL of the diluted compounds is acoustically dispensed into a 384-well plate (Corning, catalog #3820) using an Echo 550 (Labcyte).

Each well receives 6 μL of Biotinylated KRAS G12C assay solution using a BioRAPTR (Beckman Coulter) and is incubated at room temperature for 60 minutes in a humidified chamber. Each well then receives 3 μL of 120 nM recombinant human SOS protein and 9 mM GTP (Sigma, G8877) in assay buffer and is incubated at room temperature for 60 minutes in a humidified chamber.

The time-resolved fluorescence resonance energy transfer signal of both plates is measured on an Envision (PerkinElmer) plate reader: Excitation filter=340 nm; emission 1=495 nm; emission 2=520 nm; dichroic mirror=D400/D505; delay time=100 μs. The signal of each well is determined as the ratio of the emission at 520 nm to that at 495 nm. Percent effect of each well is determined after normalization to control wells containing DMSO (no effect) or a saturating concentration of inhibitor (max effect). The apparent effect as a function of compound concentration is fit to a four-parameter logistic equation. Table B shows the results.

### Test Example 5: Procedure for RAS:RAF-RBD Binding Assay

The RAF-Ras binding domain (RBD) protein interaction assay utilizes Biotinylated KRAS G12C protein (SEQ ID NO: 3) and the GST-tagged Ras binding domain of c-RAF (residues 50-132) from Jena Biosciences). Compounds are added to KRAS and then after a 30-minute incubation time the RAF-RBD and detection antibodies are added. Small molecule inhibitors that block the interaction of c-RAF-RBD prevent generation of a TR-FRET signal.

Biotinylated KRAS G12C protein is diluted to 20 nM in assay buffer (20 mM HEPES pH 7.5, 150 mM sodium chloride, 10 mM magnesium chloride, and 0.01% Tween20).

Each test compound (10 mM stock in DMSO) is diluted in DMSO to make a 10-point, 3-fold dilution series in a 384-well low dead volume microplate (Labcyte, catalog #LP-0200). Once titrations are made, 50 nL of the diluted compounds is acoustically dispensed into 384-well plates (Corning, catalog #3820) using an Echo 655 (Labcyte).

Each well of the assay plate receives 5 μL of Biotinylated KRAS G12C assay solution and is incubated at room temperature for 30 minutes. Each well then receives 5 μL of 100 nM GST-c-RAF RBD protein and a 1:100 dilution of both anti-GST-d2 (Cisbio catalog #61GSTDLA) and Strepavidin-Tb cryptate (Cisbio catalog #610SATLA) in assay buffer and the plate is mixed and briefly centrifuged followed by a 60 minute incubation at room temperature.

The time-resolved fluorescence resonance energy transfer signal of both plates is measured on an Envision (PerkinElmer) plate reader: dichroic mirror=LANCE/DELFIA DUAL/Bias; Emission1=615 nm; Emission2=665 nm; delay time=60 s. The signal of each well is determined as the ratio of the emission at 665 nm to that at 615 nm. Percent effect of each well is determined after normalization to control wells containing DMSO (no effect) or a saturating concentration of inhibitor (max effect). The apparent effect as a function of compound concentration is fit to a four-parameter logistic equation. Table B shows the results.

TABLE B
Biochemical and Cellular Data

| | Test Example 4 | Test Example 5 |
|---|---|---|
| Example | GNE IC₅₀ | cRaf IC₅₀ |
| 22 | 5.6 | 122 |
| 23a | 131 | >50000 |
| 23b | 16 | 12670 |
| 24 | 55 | >50000 |
| 25a | 142 | |
| 25b | 1.6 | 74 |
| 26 | 80.81 | 48780 |

Sequences
SEQ ID NO: 1
MASSHHHHHHSSENLYFQGMTEYKLVVVGACGVGKSALTIQLIQNHFVDE
YDPTIEDSYRKQVVIDGETCLLDILDTAGQEEYSAMRDQYMRTGEGFLCV
FAINNTKSFEDIHHYREQIKRVKDSEDVPMVLVGNKCDLPSRTVDTKQAQ
DLARSYGIPFIETSAKTRQGVDDAFYTLVREIRKHKEK
SEQ ID NO: 2
GEEQMRLPSADVYRFAEPDSEENIIFEENMQPKAGIPIIKAGTVIKLIER
LTYHMYADPNFVRTFLTTYRSFCKPQELLSLIIERFEIPEPEPTEADRIA
IENGDQPLSAELKRFRKEYIQPVQLRVLNVCRHWVEHHFYDFERDAYLLQ
RMEEFIGTVRGKAMKKWVESITKIIQRKKIARDNGPGHNITFQSSPPTVE
WHISRPGHIETFDLLTLHPIEIARQLTLLESDLYRAVQPSELVGSVWTKE
DKEINSPNLLKMIRHTTNLTLWFEKCIVETENLEERVAVVSRIIEILQVF
QELNNFNGVLEVVSAMNSSPVYRLDHTFEQIPSRQKKILEEAHELSEDHY
KKYLAKLRSINPPCVPFFGIYLTNILKTEEGNPEVLKRHGKELINFSKRR
KVAEITGEIQQYQNQPYCLRVESDIKRFFENLNPMGNSMEKEFTDYLFNK
SLEIEPRNPKPLPRFPKKYSYPLKSPGVRPSNPRPGT
SEQ ID NO: 3
GGIFEAQKIEWHETEYKLVVVGACGVGKSALTIQLIQNHFVDEYDPTIED
SYRKQVVIDGETSLLDILDTAGQEEYSAMRDQYMRTGEGFLLVFAINNTK
SFEDIHHYREQIKRVKDSEDVPMVLVGNKSDLPSRTVDTKQAQDLARSYG
IPFIETSAKTRQGVDDAFYTLVREIRKHKEK

1. A compound of the Formula (I) 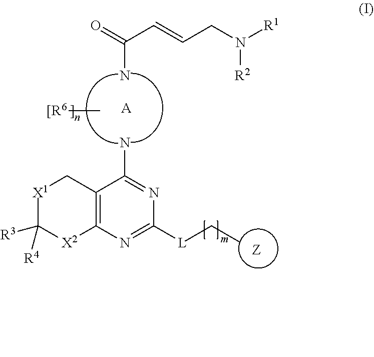
wherein:
one of X¹ and X² is O and the other is C(R⁵);
R⁵ is H;
R³ is ring C³, wherein ring C³ is selected from the group consisting of
(i) a 9- or 10-membered bicyclic aryl containing 0 to 3 ring atoms selected from the group consisting of N, O, and S;
(ii) phenyl; and
(iii) pyridyl;
wherein ring C³ is unsubstituted or substituted by 1 to 4 R^{C3} substituents independently selected from the group consisting of halo, C₁-C₃ alkyl, C₁-C₃ fluoroalkyl, C₂-C₄ alkenyl, C₂-C₄ alkynyl, C₁-C₃ alkoxy, hydroxy, amino, and cyano;
R⁴ is H;
or alternatively, R³ and R⁴ together with the carbon atom to which they are attached form a group 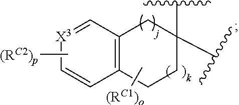
X³ is C(H) or N;
R^{C1} is independently selected from the group consisting of fluoro, C₁-C₃ alkyl, and C₁-C₃ fluoroalkyl;
R^{C2} is independently selected from the group consisting of halo, C₁-C₃ alkyl, C₁-C₃ fluoroalkyl, C₂-C₄ alkynyl, C₁-C₃ alkoxy, hydroxy, amino, and cyano;
subscript j is 0 or 1;
subscript k is 0, 1 or 2;
subscript o is 0, 1, 2, or 3;
subscript p is 0, 1, 2, 3, or 4;
A is a 5- to 8-membered monocyclic or 7- to 9-membered bicyclic saturated heterocyclic ring containing 0 to 1 additional heteroatoms selected from the group consisting of N, O, and S;
R¹ and R² are independently selected from the group consisting of:
H;
C₁-C₆ alkyl;
C₁-C₆ fluoroalkyl; and
a 3- to 8-membered mono- or bridged bicyclic saturated ring containing 0 to 2 heteroatoms selected from the group consisting of N, O, and S;
wherein the 3- to 8-membered mono- or bridged bicyclic saturated ring is unsubstituted or substituted by 1 to 4 R^{1a} substituents independently selected from the group consisting of fluoro, C₁-C₃ alkyl, C₁-C₃ fluoroalkyl, C₁-C₃ alkoxy, hydroxy, and C₁-C₃ hydroxyalkyl;
or alternatively, R¹ and R² together with the N atom to which they are attached form a 5- to 8-membered saturated heterocyclic ring having 0 to 1 additional heteroatoms selected from the group consisting of N, O, and S;
wherein the 5- to 8-membered saturated heterocyclic ring is unsubstituted or substituted by 1 to 4 Rib substituents independently selected from the group consisting of halo, C₁-C₃ alkyl, C₁-C₃ fluoroalkyl, C₁-C₃ alkoxy, hydroxy, and C₁-C₃ hydroxyalkyl;
R⁶ is selected from the group consisting of halo, C₁-C₃ alkyl, C₁-C₃ fluoroalkyl, C₁-C₄ cyanoalkyl, and C₁-C₃ hydroxyalkyl;
Ring Z is selected from the group consisting of
(i) a 3- to 10-membered mono- or bicyclic cycloalkyl;
(ii) a 3- to 10-membered mono- or bicyclic heterocycloalkyl, wherein said heterocycloalkyl is saturated and contains 1 to 2 heteroatoms selected from the group consisting of N, S, and O; and
(iii) a 3- to 8-membered spiroheterocycloalkyl, wherein said spiroheterocycloalkyl is saturated and contains 1 to 2 heteroatoms selected from the group consisting of N, S, and O;
wherein Ring Z is unsubstituted or substituted by 1 to 4 R^{Z} substituents independently selected from the group consisting of fluoro, hydroxy, C₁-C₆ alkyl, C₁-C₃ alkoxy, C₁-C₃ hydroxyalkyl, C₁-C₃ alkylsulfinyl, C₁-C₃ alkylsulfonyl, C₁-C₃ fluoroalkyl, methoxy(C₁-C₃)alkyl, carboxy, (R^{a})₂NC(O)—, (R^{a})₂NC(O)(C₁-C₃) alkyl, (R^{a})₂N(C₁-C₃)alkyl, and (R^{a})₂NC(O)—O—(C₁-C₃) alkyl;
each occurrence of R^{a} is independently H or C₁-C₃ alkyl;
Ring Z is optionally substituted by 1-M-R^{ZC}, wherein
M is —CH₂— or absent; and
R^{ZC} is a 5- to 6-membered mono- or a 9- to 10-membered bicyclic saturated heterocycloalkyl which contains 1 to 3 heteroatoms selected from the group consisting of N, S, and O, wherein R^{ZC} is unsubstituted or substituted by 1-3 substituents independently selected from the group consisting of C₁-C₃ alkyl, C₁-C₃ alkylcarbonylalkyl, C₁-C₃ hydroxyalkyl, fluoro, cyano, (R^{a})₂N—, C₁-C₃ alkoxyalkyl, (R^{a})₂NC(O) (C₁-C₃) alkyl, and C₁-C₄ cyanoalkyl;
L is O or absent;
subscript m is 0, 1, or 2; and
subscript n is 0, 1, 2, or 3; or
a pharmaceutically acceptable salt thereof.

2. The compound of claim 1 or a pharmaceutically acceptable salt thereof, wherein ring A is piperazine.

3. The compound of claim 1 or the pharmaceutically acceptable salt thereof, wherein R⁶ is —CH₂CN and subscript n is 1.

4. The compound of claim 1 or the pharmaceutically acceptable salt thereof, wherein the group 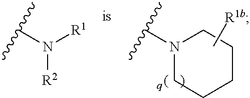
R^{1b} is C₁-C₃ alkoxy, hydroxy, or C₁-C₃ hydroxyalkyl; and
subscript q is 1, 2, or 3.

5. The compound of claim 1 or the pharmaceutically acceptable salt thereof, wherein the group

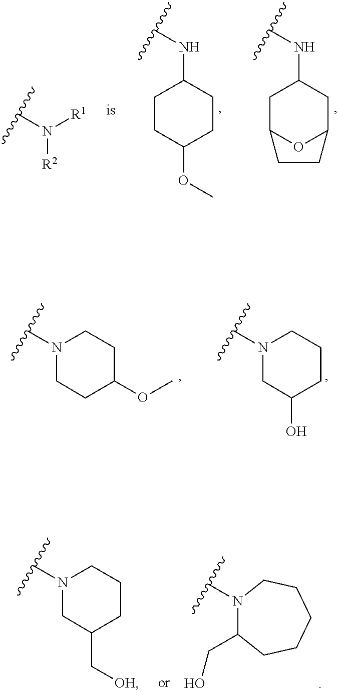

6. The compound of claim 1 or the pharmaceutically acceptable salt thereof, wherein the moiety

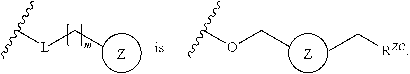

7. The compound of claim 1 or the pharmaceutically acceptable salt thereof, wherein the moiety

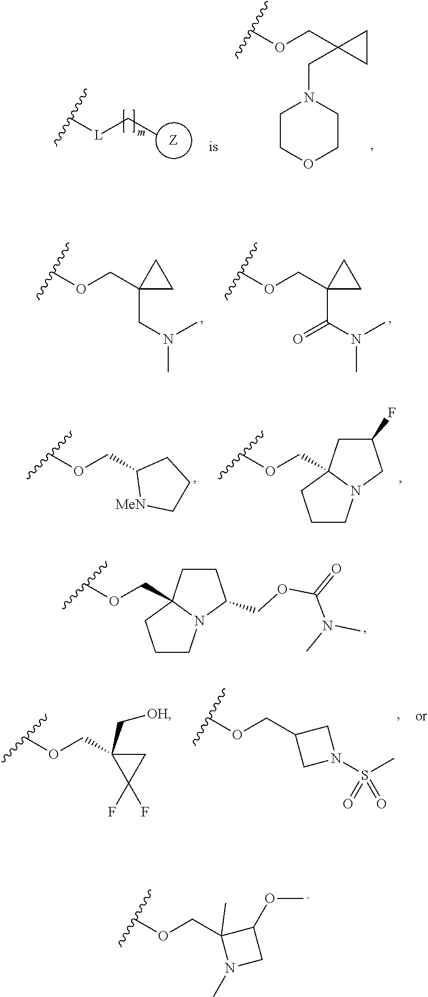

8. The compound of claim 1 or a pharmaceutically acceptable salt thereof, wherein X¹ is O and X² is C(R⁵).

9. The compound of claim 1 or a pharmaceutically acceptable salt thereof, wherein R³ is unsubstituted naphthyl or naphthyl substituted by 1 to 4 R^{C3} substituents; and R⁴ is H.

10. The compound of claim 9 or a pharmaceutically acceptable salt thereof, wherein R³ is

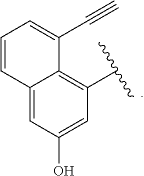 [SMILES: *c1cc(O)cc2cccc(C#C)c12]

11. The compound of claim 1 or a pharmaceutically acceptable salt thereof, wherein R³ and R⁴, together with the carbon atom to which they are attached, form a group

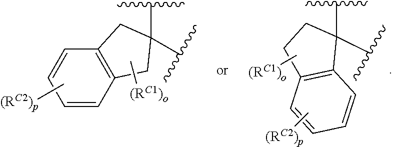

12. The compound of claim 1 or a pharmaceutically acceptable salt thereof, wherein the compound of Formula (I) has the Formula (IA)

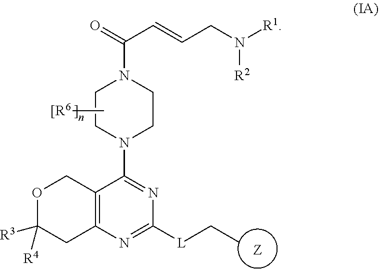

13. The compound of claim 12 or a pharmaceutically acceptable salt thereof, wherein the group 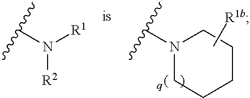
R^{1b} is C₁-C₃ alkoxy, hydroxy, or C₁-C₃ hydroxyalkyl;
subscript q is 1, 2, or 3;
R⁶ is —CH₂CN and subscript n is 1;
the moiety 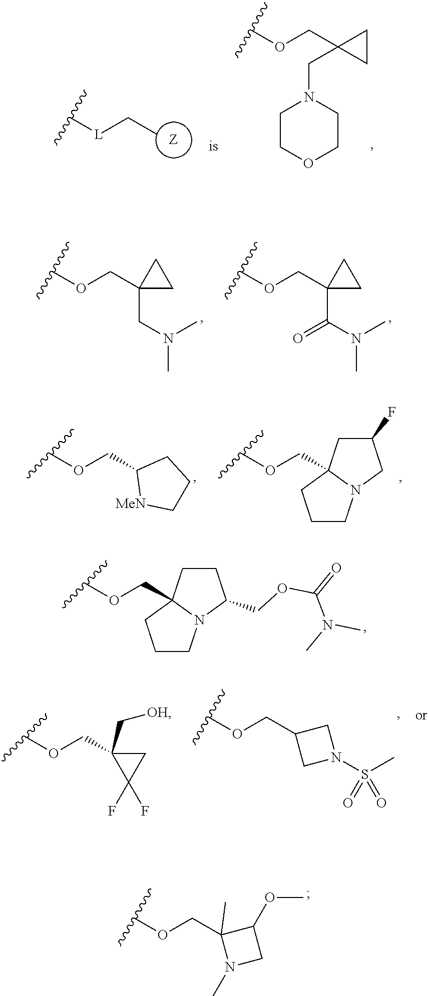
R³ is 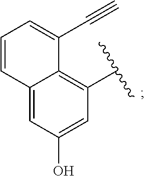 [SMILES: *c1cc(O)cc2cccc(C#C)c12]
and
R⁴ is H.

14. The compound of claim 1 selected from Examples 1-26 or the pharmaceutically acceptable salt thereof.

15. The compound of claim 1 or a pharmaceutically acceptable salt thereof, wherein the compound is selected from the group consisting of:

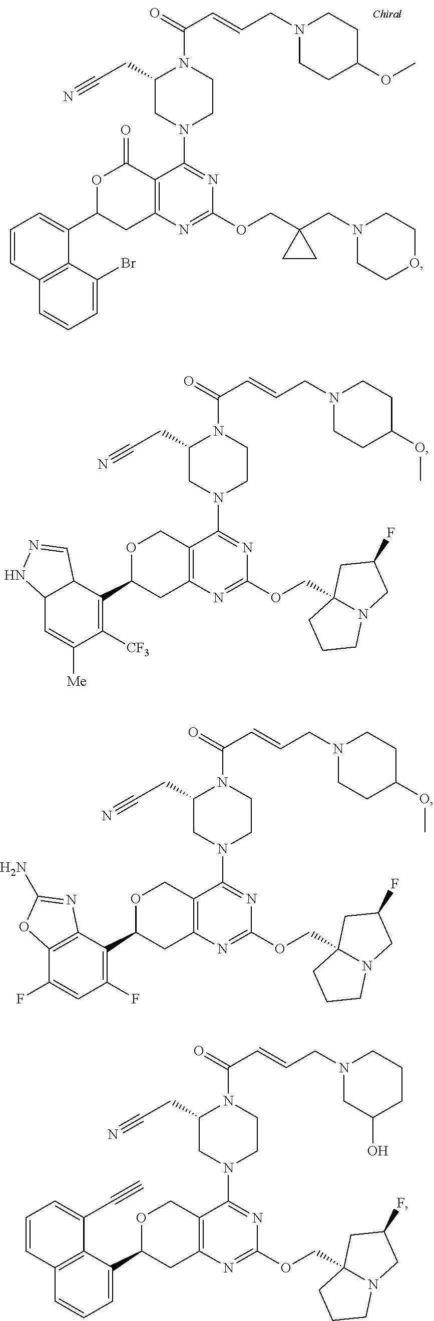 [SMILES: COC1CCN(C/C=C/C(=O)N2CCN(c3nc(OCC4(CN5CCOCC5)CC4)nc4c3C(=O)OC(c3cccc5cccc(Br)c35)C4)C[C@@H]2CC#N)CC1 ; COC1CCN(C/C=C/C(=O)N2CCN(c3nc(OC[C@@]45CCCN4C[C@H](F)C5)nc4c3CO[C@H](C3=C(C(F)(F)F)C(C)=CC5NN=CC35)C4)C[C@@H]2CC#N)CC1 ; COC1CCN(C/C=C/C(=O)N2CCN(c3nc(OC[C@@]45CCCN4C[C@H](F)C5)nc4c3CO[C@H](c3c(F)cc(F)c5oc(N)nc35)C4)C[C@@H]2CC#N)CC1 ; C#Cc1cccc2cccc([C@@H]3Cc4nc(OC[C@@]56CCCN5C[C@H](F)C6)nc(N5CCN(C(=O)/C=C/CN6CCCC(O)C6)[C@@H](CC#N)C5)c4CO3)c12]

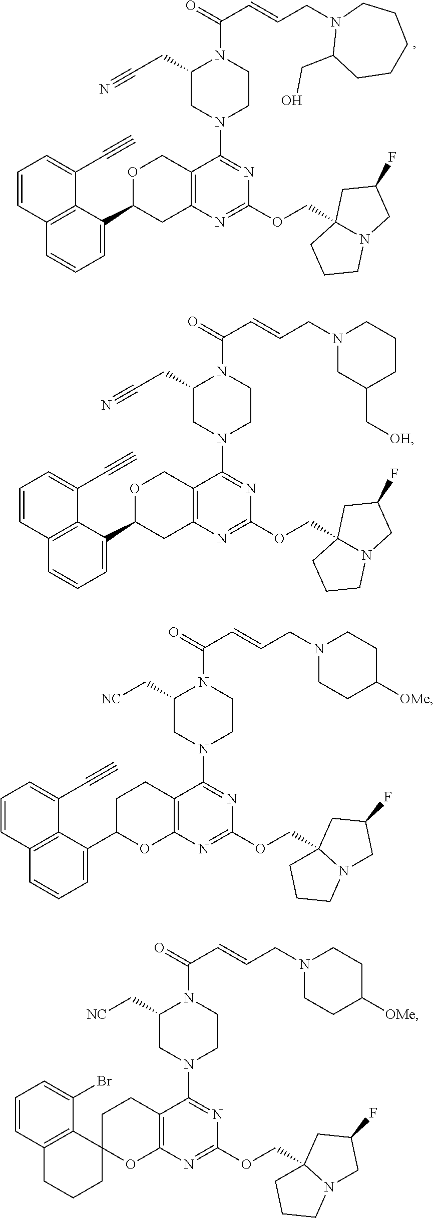

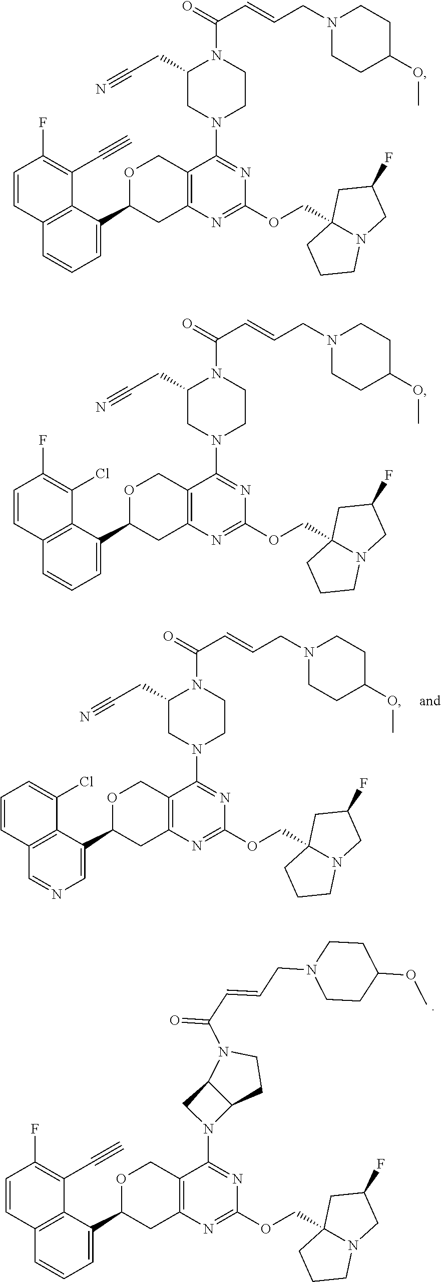

16. A pharmaceutical composition comprising the compound of claim 1 or the pharmaceutically acceptable salt thereof, and a pharmaceutically acceptable carrier.

17. A method of inhibiting KRAS G12C protein comprising contacting KRAS G12C protein with the compound of claim 1, or the pharmaceutically acceptable salt thereof, to inhibit the activity of the KRAS G12C protein.

18. A method of treating cancer comprising administering a therapeutically effective amount of the compound of claim 1, or the pharmaceutically acceptable salt thereof, to a subject in need of such treatment.

19. The method of claim 18, further comprising administering an additional active agent to the subject.

20.-26. (canceled)


SEQUENCE LISTING (3 sequences)

SEQ ID NO: 1 - protein, 188 aa, Artificial
  Synthetic Protein
MASSHHHHHHSSENLYFQGMTEYKLVVVGACGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 2 - protein, 487 aa, Artificial
  Synthetic Protein
GEEQMRLPSADVYRFAEPDSEENIIFEENMQPKAGIPIIKAGTVIKLIERLTYHMYADPNFVRTFLTTYRSFCKPQELLSLIIERFEIPEPEPTEADRIAIENGDQPLSAELKRFRKEYIQPVQLRVLNVCRHWVEHHFYDFERDAYLLQRMEEFIGTVRGKAMKKWVESITKIIQRKKIARDNGPGHNITFQSSPPTVEWHISRPGHIETFDLLTLHPIEIARQLTLLESDLYRAVQPSELVGSVWTKEDKEINSPNLLKMIRHTTNLTLWFEKCIVETENLEERVAVVSRIIEILQVFQELNNFNGVLEVVSAMNSSPVYRLDHTFEQIPSRQKKILEEAHELSEDHYKKYLAKLRSINPPCVPFFGIYLTNILKTEEGNPEVLKRHGKELINFSKRRKVAEITGEIQQYQNQPYCLRVESDIKRFFENLNPMGNSMEKEFTDYLFNKSLEIEPRNPKPLPRFPKKYSYPLKSPGVRPSNPRPGT

SEQ ID NO: 3 - protein, 181 aa, Artificial
  Synthetic Protein
GGIFEAQKIEWHETEYKLVVVGACGVGKSALTIQLIQNHFVDEYDPTIEDSYRKQVVIDGETSLLDILDTAGQEEYSAMRDQYMRTGEGFLLVFAINNTKSFEDIHHYREQIKRVKDSEDVPMVLVGNKSDLPSRTVDTKQAQDLARSYGIPFIETSAKTRQGVDDAFYTLVREIRKHKEK